(19) United States
(12) Patent Application Publication
(10) Pub. No.: US 2025/0295901 A1
(43) Pub. Date: Sep. 25, 2025

(54) FLEXIBLE LINE CONFIGURATIONS FOR GASTRIC RESIDENCE SYSTEMS

(71) Applicant: Lyndra Therapeutics, Inc., Watertown, MA (US)

(72) Inventors: Rosemary KANASTY, Somerville, MA (US); Rachel DERECH, Cambridge, MA (US); Yi SHI, Natick, MA (US); Zhiyu XIA, Watertown, MA (US); Carmi MANDELKERN, Cambridge, MA (US); Benjamin DICKSTEIN, Wayland, MA (US); Roman LOVELL, North Reading, MA (US); Kiera HENDERSON, Watertown, MA (US); Erick PEEKE, Cambridge, MA (US)

(21) Appl. No.: 19/082,659

(22) Filed: Mar. 18, 2025

Related U.S. Application Data
(60) Provisional application No. 63/567,323, filed on Mar. 19, 2024.
(60) Provisional application No. 63/572,128, filed on Mar. 29, 2024.

(51) Int. Cl. A61M 31/00

(52) U.S. Cl. CPC A61M 31/002; A61M 2205/332; A61M 2207/00; A61M 2210/1053

(57) ABSTRACT

A gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm, wherein the flexible line comprises an irregular cross-section.

### CROSS-REFERENCE TO RELATED APPLICATIONS

This application claims priority benefit of U.S. Provisional Patent Application No. 63/567,323 filed Mar. 19, 2024 and U.S. Provisional Patent Application No. 63/572,128 filed Mar. 29, 2024. The entire contents of that application are hereby incorporated by reference herein.

### FIELD

The present disclosure relates generally to gastric residence systems, and more specifically to flexible line configurations for gastric residence systems.

### BACKGROUND

Gastric residence systems are delivery systems for agents which remain in the stomach for days to weeks, or over even longer periods, during which time drugs or other agents can elute from the systems for absorption in the gastrointestinal tract. Examples of such systems are described in International Patent Application Nos. WO 2015/191920, WO 2015/191925, WO 2017/070612, WO 2017/100367, WO 2017/205844, and WO 2018/227147.

Gastric residence systems can be administered to a patient using capsules which are swallowed or introduced into the stomach of the patient by an alternate method (e.g., via a feeding tube or a gastric tube). Upon dissolution of a capsule in the stomach, a gastric residence system may expand or unfold to a size which remains in the stomach and resists passage through the pylorus over a desired gastric residence period. Throughout the desired residence period, the system elutes one or more agents (e.g., drugs) at a desired rate. At the end of the residence period, the system passes through the pylorus and is eliminated from the patient. If the system passes through the pylorus before the end of the desired residence period, however, the one or more agents may not be delivered to the patient as intended. The present disclosure provides flexible line configurations for gastric retention systems, which help to prevent premature passage of the systems through the pylorus.

### SUMMARY

Provided herein are flexible line configurations for gastric residence systems. Including one or more flexible lines in a gastric residence system can improve gastric residence by preventing the gastric residence system or a portion thereof from prematurely passing through the pylorus. The one or more flexible lines can maintain the structure of a gastric residence system in its expanded configuration, thereby increasing the force required to compress the gastric residence system into a configuration small enough to pass through the pylorus. Preventing premature passage helps to ensure that the agent in the gastric residence system is delivered to the patient as intended over the desired gastric residence period.

In some embodiments, a gastric residence system is provided, the gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm, wherein the flexible line comprises an irregular cross-section.

In some embodiments, the irregular cross-section comprises a braided cross-section. In some embodiments, the irregular cross-section comprises at least one of bumps, ridges, knots, barbs, or valleys. In some embodiments, the plurality of arms comprises at least three arms. In some embodiments, two or three arms comprise an active pharmaceutical ingredient-containing portion and a separate tip portion at a distal end of the two or three arms. In some embodiments, the active pharmaceutical ingredient comprises 10-80% by weight of the active pharmaceutical ingredient-containing portion. In some embodiments, the active pharmaceutical ingredient comprises 40-60% by weight of the active pharmaceutical ingredient-containing portion. In some embodiments, the active pharmaceutical ingredient comprises 20-40% by weight of the active pharmaceutical ingredient-containing portion. In some embodiments, each arm of the plurality of arms comprises a triangular cross-section. In some embodiments, the triangular cross-section has a base of 2.5-3.5 mm and a height of 2.1-3.1 mm. In some embodiments, the triangular cross-section has a base of 2.9-3.3 mm and a height of 2.5-2.9 mm. In some embodiments, the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, 100% poly-L-lactide, polyethylene terephthalate, polypropylene, polyamide, polyester, poly(ether ester), polytetrafluoroethylene, or polyvinylidinefluoride (PVDF). In some embodiments, the flexible line comprises one or more of poly(glycolic acid) (PGA), poly(lactic-co-glycolic acid) (PLGA), or polyethylene terephthalate (PET). In some embodiments, the flexible line has a diameter of 0.05-1 mm. In some embodiments, the flexible line has a cross-sectional area of 0.005-3.14 mm². In some embodiments, the flexible line has a cross-sectional area of 0.1-5 mm². In some embodiments, the flexible line has a cross-sectional area of 0.5-3.5 mm². In some embodiments, the flexible line has a cross-sectional area of 0.6-1 mm². In some embodiments, the flexible line has a surface roughness of 5-400 μm. In some embodiments, the flexible line has a surface roughness of 50-250 μm. In some embodiments, the flexible line comprises a braid of at least three strands. In some embodiments, the braid has a braid angle of 5-40 degrees and a braid length of 0.5-3.1 mm. In some embodiments, the flexible line is absorbable. In some embodiments, the flexible line is non-absorbable. In some embodiments, the separate tip portion comprises polycaprolactone. In some embodiments, the separate tip portion further comprises copovidone. In some embodiments, the separate tip portion further comprises Poloxamer 407. In some embodiments, the separate tip portion further comprises a colorant. In some embodiments, the separate tip portion further comprises bismuth subcarbonate. In some embodiments, the distal end of each arm comprises polycaprolactone. In some embodiments, the distal end of each arm further comprises copovidone. In some embodiments, the distal end of each arm further comprises Poloxamer 407. In some embodiments, the distal end of each arm further comprises a colorant. In some embodiments, the distal end of each arm further comprises bismuth subcarbonate. In some embodiments, the separate tip portion has a flexural modulus of at least 50 MPa. In some embodiments, the distal end of each arm has a flexural modulus of at least 50 MPa. In some embodiments, the separate tip portion has a flexural strength of at least 5 N. In some embodiments, the separate tip portion has a flexural strength of at least 8 N. In some embodiments, the distal end of each arm has a flexural strength of at least 5 N. In some embodiments, the distal end of each arm has a flexural strength of at least 8 N. In some embodiments, the separate tip portion has a maximum force of at least 3 N. In some embodiments, the separate tip portion has a maximum force of at least 5 N. In some embodiments, the distal end of each arm has a maximum force of at least 3 N. In some embodiments, the distal end of each arm has a maximum force of at least 5 N. In some embodiments, the separate tip portion softens less than 80% upon hydration. In some embodiments, the separate tip portion softens more than 10% upon hydration. In some embodiments, the distal end of each arm softens less than 80% upon hydration. In some embodiments, the distal end of each arm softens more than 10% upon hydration. In some embodiments, the flexible line is embedded in the distal end of each arm by heating the distal end, pressing a portion of the flexible line into the distal end, and cooling the distal end with the portion of the flexible line embedded. In some embodiments, heating the distal end comprises directing a laser at the distal end. In some embodiments, the heating comprises convection heating. In some embodiments, the laser comprises an infrared laser. In some embodiments, a force required to release the flexible line from a first arm of the plurality of arms is at least 4N. In some embodiments, a force required to release the flexible line from a first arm of the plurality of arms is at least 8N. In some embodiments, the gastric residence system is configured to be folded during administration and is configured to assume an open configuration when in a patient's stomach. In some embodiments, the gastric residence system has a multi-armed star shape in the open configuration. In some embodiments, the gastric residence system comprises a core, wherein each arm of the plurality of arms is connected to the core at the proximal end of each arm. In some embodiments, the core undergoes elastic deformation when the gastric residence system is in the folded configuration and recoils when the gastric residence system assumes the open configuration. In some embodiments, the gastric residence system comprises a plurality of linker components, wherein one linker component of the plurality of linker components connects one arm of the plurality of arms to the core. In some embodiments, each linker component of the plurality of linker components degrades, dissolves, disassociates, or mechanically weakens in a gastric environment. In some embodiments, the gastric residence system is used to treat a patient. In some embodiments, the patient is a human.

In some embodiments, a gastric residence system comprises: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, 100% poly-L-lactide, polyethylene terephthalate, polypropylene, polyamide, polyester, poly(ether ester), polytetrafluoroethylene, or polyvinylidinefluoride (PVDF).

In some embodiments, the flexible line comprises an irregular cross-section. In some embodiments, the irregular cross-section comprises a braided cross-section. In some embodiments, the irregular cross-section comprises at least one of bumps, ridges, knots, barbs, or valleys. In some embodiments, the plurality of arms comprises at least three arms. In some embodiments, two or three arms comprise an active pharmaceutical ingredient-containing portion and a separate tip portion at a distal end of the two or three arms. In some embodiments, the active pharmaceutical ingredient comprises 10-80% by weight of the active pharmaceutical ingredient-containing portion. In some embodiments, the active pharmaceutical ingredient comprises 40-60% by weight of the active pharmaceutical ingredient-containing portion. In some embodiments, the active pharmaceutical ingredient comprises 20-40% by weight of the active pharmaceutical ingredient-containing portion. In some embodiments, each arm of the plurality of arms comprises a triangular cross-section. In some embodiments, the triangular cross-section has a base of 2.5-3.5 mm and a height of 2.1-3.1 mm. In some embodiments, the triangular cross-section has a base of 2.9-3.3 mm and a height of 2.5-2.9 mm. In some embodiments, the flexible line has a diameter of 0.05-1 mm. In some embodiments, the flexible line has a cross-sectional area of 0.005-3.14 mm². In some embodiments, the flexible line has a cross-sectional area of 0.1-5 mm². In some embodiments, the flexible line has a cross-sectional area of 0.5-3.5 mm². In some embodiments, the flexible line has a cross-sectional area of 0.6-1 mm². In some embodiments, the flexible line has a surface roughness of 5-400 μm. In some embodiments, the flexible line has a surface roughness of 50-250 μm. In some embodiments, the flexible line comprises a braid of at least three strands. In some embodiments, the braid has a braid angle of 5-40 degrees and a braid length of 0.5-3.1 mm. In some embodiments, the flexible line is absorbable. In some embodiments, the flexible line is non-absorbable. In some embodiments, the separate tip portion comprises polycaprolactone. In some embodiments, the separate tip portion further comprises copovidone. In some embodiments, the separate tip portion further comprises Poloxamer 407. In some embodiments, the separate tip portion further comprises a colorant. In some embodiments, the separate tip portion further comprises bismuth subcarbonate. In some embodiments, the distal end of each arm comprises polycaprolactone. In some embodiments, the distal end of each arm further comprises copovidone. In some embodiments, the distal end of each arm further comprises Poloxamer 407. In some embodiments, the distal end of each arm further comprises a colorant. In some embodiments, the distal end of each arm further comprises bismuth subcarbonate. In some embodiments, the separate tip portion has a flexural modulus of at least 50 MPa. In some embodiments, the distal end of each arm has a flexural modulus of at least 50 MPa. In some embodiments, the separate tip portion has a flexural strength of at least 5 N. In some embodiments, the separate tip portion has a flexural strength of at least 8 N. In some embodiments, the distal end of each arm has a flexural strength of at least 5 N. In some embodiments, the distal end of each arm has a flexural strength of at least 8 N. In some embodiments, the separate tip portion has a maximum force of at least 3 N. In some embodiments, the separate tip portion has a maximum force of at least 5 N. In some embodiments, the distal end of each arm has a maximum force of at least 3 N. In some embodiments, the distal end of each arm has a maximum force of at least 5 N. In some embodiments, the separate tip portion softens less than 80% upon hydration. In some embodiments, the separate tip portion softens more than 10% upon hydration. In some embodiments, the distal end of each arm softens less than 80% upon hydration. In some embodiments, the distal end of each arm softens more than 10% upon hydration. In some embodiments, the flexible line is embedded in the distal end of each arm by heating the distal end, pressing a portion of the flexible line into the distal end, and cooling the distal end with the portion of the flexible line embedded. In some embodiments, heating the distal end comprises directing a laser at the distal end. In some embodiments, the heating comprises convection heating. In some embodiments, the laser comprises an infrared laser. In some embodiments, a force required to release the flexible line from a first arm of the plurality of arms is at least 4N. In some embodiments, a force required to release the flexible line from a first arm of the plurality of arms is at least 8N. In some embodiments, the gastric residence system is configured to be folded during administration and is configured to assume an open configuration when in a patient's stomach. In some embodiments, the gastric residence system has a multi-armed star shape in the open configuration. In some embodiments, each arm of the plurality of arms is connected to the core at the proximal end of each arm. In some embodiments, the core undergoes elastic deformation when the gastric residence system is in the folded configuration and recoils when the gastric residence system assumes the open configuration. In some embodiments, the gastric residence system comprises a plurality of linker components, wherein one linker component of the plurality of linker components connects one arm of the plurality of arms to the core. In some embodiments, each linker component of the plurality of linker components degrades, dissolves, disassociates, or mechanically weakens in a gastric environment. In some embodiments, the gastric residence system is used to treat a patient. In some embodiments, the patient is a human.

In some embodiments, a gastric residence system comprises: a plurality of arms connected at a proximal end of each arm, the plurality of arms extending radially from the proximal ends, at least one arm comprising an active pharmaceutical ingredient-containing portion; and a flexible line connecting the plurality of arms, wherein a respective portion of the flexible line is embedded within a distal end of each arm, wherein the flexible line comprises an irregular cross-section.

In some embodiments, the active pharmaceutical ingredient comprises 10-80% by weight of the active pharmaceutical ingredient-containing portion. In some embodiments, the active pharmaceutical ingredient comprises 40-60% by weight of the active pharmaceutical ingredient-containing portion. In some embodiments, the active pharmaceutical ingredient comprises 20-40% by weight of the active pharmaceutical ingredient-containing portion. In some embodiments, the flexible line comprises an irregular cross-section. In some embodiments, the irregular cross-section comprises a braided cross-section. In some embodiments, the irregular cross-section comprises at least one of bumps, ridges, knots, barbs, or valleys. In some embodiments, the plurality of arms comprises at least three arms. In some embodiments, each arm of the plurality of arms comprises a triangular cross-section. In some embodiments, the triangular cross-section has a base of 2.5-3.5 mm and a height of 2.1-3.1 mm. In some embodiments, the triangular cross-section has a base of 2.9-3.3 mm and a height of 2.5-2.9 mm. In some embodiments, the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, 100% poly-L-lactide, polyethylene terephthalate, polypropylene, polyamide, polyester, poly(ether ester), polytetrafluoroethylene, or polyvinylidinefluoride (PVDF). In some embodiments, the flexible line comprises one or more of poly(glycolic acid) (PGA), poly(lactic-co-glycolic acid) (PLGA), or polyethylene terephthalate (PET). In some embodiments, the flexible line has a diameter of 0.05-1 mm. In some embodiments, the flexible line has a cross-sectional area of 0.005-3.14 mm². In some embodiments, the flexible line has a cross-sectional area of 0.5-1 mm². In some embodiments, the flexible line has a cross-sectional area of 0.5-3.5 mm². In some embodiments, the flexible line has a cross-sectional area of 0.6-1 mm². In some embodiments, the flexible line has a surface roughness of 5-400 μm. In some embodiments, the flexible line has a surface roughness of 50-250 μm. In some embodiments, the flexible line comprises a braid of at least three strands. In some embodiments, the braid has a braid angle of 5-40 degrees and a braid length of 0.5-3.1 mm. In some embodiments, the flexible line is absorbable. In some embodiments, the flexible line is non-absorbable. In some embodiments, the distal end of each arm comprises polycaprolactone. In some embodiments, the distal end of each arm further comprises copovidone. In some embodiments, the distal end of each arm further comprises Poloxamer 407. In some embodiments, the distal end of each arm further comprises a colorant. In some embodiments, the distal end of each arm further comprises bismuth subcarbonate. In some embodiments, the distal end of each arm has a flexural modulus of at least 50 MPa. In some embodiments, the distal end of each arm has a flexural strength of at least 5 N. In some embodiments, the distal end of each arm has a flexural strength of at least 8 N. In some embodiments, the distal end of each arm has a maximum force of at least 3 N. In some embodiments, the distal end of each arm has a maximum force of at least 5 N. In some embodiments, the distal end of each arm softens less than 80% upon hydration. In some embodiments, the distal end of each arm softens more than 10% upon hydration. In some embodiments, the at least one arm comprising an active pharmaceutical ingredient-containing portion comprises a separate tip portion at a distal end of the at least one arm. In some embodiments, two or three arms comprise an active pharmaceutical ingredient-containing portion and a separate tip portion at a distal end of the two or three arms. In some embodiments, the separate tip portion comprises polycaprolactone. In some embodiments, the separate tip portion further comprises copovidone. In some embodiments, the separate tip portion further comprises Poloxamer 407. In some embodiments, the separate tip portion further comprises a colorant. In some embodiments, the separate tip portion further comprises bismuth subcarbonate. In some embodiments, the separate tip portion has a flexural modulus of at least 50 MPa. In some embodiments, the separate tip portion has a flexural strength of at least 5 N. In some embodiments, the separate tip portion has a flexural strength of at least 8 N. In some embodiments, the separate tip portion has a maximum force of at least 3 N. In some embodiments, the separate tip portion has a maximum force of at least 5 N. In some embodiments, the separate tip portion softens less than 80% upon hydration. In some embodiments, the separate tip portion softens more than 10% upon hydration. In some embodiments, the flexible line is embedded in the distal end of each arm by heating the distal end, pressing a portion of the flexible line into the distal end, and cooling the distal end with the portion of the flexible line embedded. In some embodiments, heating the distal end comprises directing a laser at the distal end. In some embodiments, the heating comprises convection heating. In some embodiments, the laser comprises an infrared laser. In some embodiments, a force required to release the flexible line from a first arm of the plurality of arms is at least 4N. In some embodiments, a force required to release the flexible line from a first arm of the plurality of arms is at least 8N. In some embodiments, the gastric residence system is configured to be folded during administration and is configured to assume an open configuration when in a patient's stomach. In some embodiments, the gastric residence system has a multi-armed star shape in the open configuration. In some embodiments, the gastric residence system comprises a core, wherein each arm of the plurality of arms is connected to the core at the proximal end of each arm. In some embodiments, the core undergoes elastic deformation when the gastric residence system is in the folded configuration and recoils when the gastric residence system assumes the open configuration. In some embodiments, the gastric residence system comprises a plurality of linker components, wherein one linker component of the plurality of linker components connects one arm of the plurality of arms to the core. In some embodiments, each linker component of the plurality of linker components degrades, dissolves, disassociates, or mechanically weakens in a gastric environment. In some embodiments, the gastric residence system is used to treat a patient. In some embodiments, the patient is a human.

In some embodiments, a method for manufacturing a gastric residence system comprises: using a mold, preparing a gastric residence system comprising a plurality of arms connected at a proximal end of each arm, the plurality of arms extending radially from the proximal ends, at least one arm comprising an active pharmaceutical ingredient-containing portion and a separate tip portion at a distal end of the at least one arm; heating a distal end of each arm to at least partially melt the respective distal end; embedding a respective portion of at least one flexible line into each melted distal end; and cooling the melted distal ends such that melted distal ends solidify with the at least one flexible line embedded.

In some embodiments, a method for manufacturing a gastric residence system comprises: using a mold, preparing a gastric residence system comprising a plurality of arms connected at a proximal end of each arm, the plurality of arms extending radially from the proximal ends; heating a distal end of each arm to at least partially melt the respective distal end; embedding a respective portion of at least one flexible line into each melted distal end; and cooling the melted distal ends such that melted distal ends solidify with the at least one flexible line embedded.

In some embodiments, heating the distal end of each arm comprises directing a laser at the distal end of each arm. In some embodiments, the heating comprises convection heating. In some embodiments, the laser comprises an infrared laser. In some embodiments, the flexible line comprises an irregular cross-section. In some embodiments, the irregular cross-section comprises a braided cross-section. In some embodiments, the irregular cross-section comprises at least one of bumps, ridges, knots, barbs, or valleys. In some embodiments, the plurality of arms comprises at least three arms. In some embodiments, two or three arms comprise an active pharmaceutical ingredient-containing portion and a separate tip portion at a distal end of the two or three arms. In some embodiments, the active pharmaceutical ingredient comprises 10-80% by weight of the active pharmaceutical ingredient-containing portion. In some embodiments, the active pharmaceutical ingredient comprises 40-60% by weight of the active pharmaceutical ingredient-containing portion. In some embodiments, the active pharmaceutical ingredient comprises 20-40% by weight of the active pharmaceutical ingredient-containing portion. In some embodiments, each arm of the plurality of arms comprises a triangular cross-section. In some embodiments, the triangular cross-section has a base of 2.5-3.5 mm and a height of 2.1-3.1 mm. In some embodiments, the triangular cross-section has a base of 2.5-2.9 mm and a height of 2.9-3.3 mm. In some embodiments, the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, 100% poly-L-lactide, polyethylene terephthalate, polypropylene, polyamide, polyester, poly(ether ester), polytetrafluoroethylene, or polyvinylidinefluoride (PVDF). In some embodiments, the flexible line comprises one or more of polyglycolic acid, poly(lactic-co-glycolic acid), or polyethylene terephthalate. In some embodiments, the flexible line has a diameter of 0.05-1 mm. In some embodiments, the flexible line has a cross-sectional area of 0.005-3.14 mm². In some embodiments, the flexible line has a cross-sectional area of 0.1-5 mm². In some embodiments, the flexible line has a cross-sectional area of 0.5-3.5 mm². In some embodiments, the flexible line has a cross-sectional area of 0.6-1 mm². In some embodiments, the flexible line has a surface roughness of 5-400 μm. In some embodiments, the flexible line has a surface roughness of 50-250 μm. In some embodiments, the flexible line comprises a braid of at least three strands. In some embodiments, the braid has a braid angle of 5-40 degrees and a braid length of 0.5-3.1 mm. In some embodiments, the flexible line is absorbable. In some embodiments, the flexible line is non-absorbable. In some embodiments, the separate tip portion comprises polycaprolactone. In some embodiments, the separate tip portion further comprises copovidone. In some embodiments, the separate tip portion further comprises poloxamer 407. In some embodiments, the separate tip portion further comprises a colorant. In some embodiments, the separate tip portion further comprises bismuth subcarbonate. In some embodiments, the distal end of each arm comprises polycaprolactone. In some embodiments, the distal end of each arm further comprises copovidone. In some embodiments, the distal end of each arm further comprises Poloxamer 407. In some embodiments, the distal end of each arm further comprises a colorant. In some embodiments, the distal end of each arm further comprises bismuth subcarbonate. In some embodiments, the separate tip portion has an elastic modulus of at least 50 MPa. In some embodiments, the distal end of each arm has an elastic modulus of at least 50 MPa. In some embodiments, the distal end of each arm has a flexural modulus of at least 50 MPa. In some embodiments, the separate tip portion has a flexural strength of at least 5 N. In some embodiments, the separate tip portion has a flexural strength of at least 8 N. In some embodiments, the distal end of each arm has a flexural strength of at least 5 N. In some embodiments, the distal end of each arm has a flexural strength of at least 8 N. In some embodiments, the separate tip portion has a maximum force of at least 3 N. In some embodiments, the separate tip portion has a maximum force of at least 5 N. In some embodiments, the distal end of each arm has a maximum force of at least 3 N. In some embodiments, the distal end of each arm has a maximum force of at least 5 N. In some embodiments, the separate tip portion softens less than 80% upon hydration. In some embodiments, the separate tip portion softens more than 10% upon hydration. In some embodiments, the distal end of each arm softens less than 80% upon hydration. In some embodiments, the distal end of each arm softens more than 10% upon hydration. In some embodiments, the flexible line is embedded in the distal end of each arm by heating the distal end, pressing a portion of the flexible line into the distal end, and cooling the distal end with the portion of the flexible line embedded. In some embodiments, heating the distal end comprises directing a laser at the distal end. In some embodiments, the heating comprises convection heating. In some embodiments, the laser comprises an infrared laser. In some embodiments, a force required to release the flexible line from a first arm of the plurality of arms is at least 4N. In some embodiments, a force required to release the flexible line from a first arm of the plurality of arms is at least 8N. In some embodiments, the gastric residence system is configured to be folded during administration and is configured to assume an open configuration when in a patient's stomach. In some embodiments, the gastric residence system has a multi-armed star shape in the open configuration. In some embodiments, the gastric residence system comprises a core, wherein each arm of the plurality of arms is connected to the core at the proximal end of each arm. In some embodiments, the core undergoes elastic deformation when the gastric residence system is in the folded configuration and recoils when the gastric residence system assumes the open configuration. In some embodiments, the gastric residence system comprises a plurality of linker components, wherein one linker component of the plurality of linker components connects one arm of the plurality of arms to the core. In some embodiments, each linker component of the plurality of linker components degrades, dissolves, disassociates, or mechanically weakens in a gastric environment. In some embodiments, the gastric residence system is used to treat a patient. In some embodiments, the patient is a human.

In some embodiments, a method for measuring strength of a flexible line of a gastric residence system comprises: receiving a gastric residence system, the gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm; detaching a first arm and a second arm from the gastric residence system, wherein the first arm and the second arm are adjacent arms of the gastric residence system connected by a piece of the flexible line; placing the first arm in a first clamp and the second arm in a second clamp, wherein the first clamp is positioned adjacent to the second clamp; moving the first clamp away from the second clamp until the piece of the flexible line releases from the first arm or the second arm; and measuring the force required to release the piece of the flexible line. In some embodiments, moving the first clamp away from the second clamp causes the flexible line to break. In some embodiments, the method further comprises measuring a force required to break the flexible line.

In some embodiments, a method for measuring strength of a flexible line of a gastric residence system comprises: receiving a gastric residence system, the gastric residence system comprising: a plurality of arms connected at a proximal end of each arm, the plurality of arms extending radially from the proximal ends; and a flexible line connecting the plurality of arms, wherein a respective portion of the flexible line is embedded within a distal end of each arm; detaching a first arm and a second arm from the gastric residence system, wherein the first arm and the second arm are adjacent arms of the gastric residence system connected by a piece of the flexible line; placing the first arm in a first clamp and the second arm in a second clamp, wherein the first clamp is positioned adjacent to the second clamp; moving the first clamp away from the second clamp until the piece of the flexible line releases from the first arm or the second arm; and measuring the force required to release the piece of the flexible line.

In some embodiments, a gastric residence system comprises: six arms affixed to a central elastomer; each arm comprising a proximal end, a distal end, and an outer surface therebetween, wherein the proximal end of each arm is attached to the central elastomer and projects radially from the central elastomer, each arm having its distal end not attached to the central elastomer component and located at a larger radial distance from the central elastomer component than the proximal end; at least one arm comprising an active pharmaceutical ingredient-containing portion, wherein the at least one arm comprises: a first inert segment; a first disintegrating matrix segment attached to the first inert segment; a second inert segment attached to the first disintegrating matrix segment; a second disintegrating matrix segment attached to the second inert segment; a third inert segment attached to the second disintegrating matrix segment; the active pharmaceutical ingredient-containing portion attached to the third inert segment; and a fourth inert segment attached to the active pharmaceutical ingredient-containing portion; and a flexible line connecting each arm, wherein a respective portion of the flexible line is embedded within a distal end of each arm.

In some embodiments, the first inert segment is attached to the central elastomer. In some embodiments, the segments are in the order listed from the proximal end to the distal end of the arm comprising an active pharmaceutical ingredient-containing portion, wherein the first inert segment is at the proximal end of the arm comprising the active pharmaceutical ingredient-containing portion, the first inert segment is attached to the central elastomer, and the fourth inert segment is at the distal end of the arm comprising the active pharmaceutical ingredient-containing portion. In some embodiments, at least one arm excludes an active pharmaceutical ingredient-containing portion. In some embodiments, the at least one arm excluding an active pharmaceutical ingredient-containing portion comprises: a first inert segment; a first disintegrating matrix segment attached to the first inert segment; a second inert segment attached to the first disintegrating matrix segment; a second disintegrating matrix segment attached to the second inert segment; a third inert segment attached to the second disintegrating matrix segment; and a fourth inert segment attached to the third inert segment. In some embodiments, the first inert segment is attached to the central elastomer. In some embodiments, the segments are in the order listed from the proximal end to the distal end of the arm excluding an active pharmaceutical ingredient-containing portion, wherein the first inert segment is at the proximal end of the arm excluding an active pharmaceutical ingredient-containing portion, the first inert segment is attached to the central elastomer, and the fourth inert segment is at the distal end of the arm excluding the active pharmaceutical ingredient-containing portion. In some embodiments, one arm comprises the active pharmaceutical ingredient-containing portion and five arms exclude the active pharmaceutical ingredient-containing portion. In some embodiments, two arms comprise the active pharmaceutical ingredient-containing portion and four arms exclude the active pharmaceutical ingredient-containing portion. In some embodiments, three arms comprise the active pharmaceutical ingredient-containing portion and three arms exclude the active pharmaceutical ingredient-containing portion. In some embodiments, four arms comprise the active pharmaceutical ingredient-containing portion and two arms exclude the active pharmaceutical ingredient-containing portion. In some embodiments, five arms comprise the active pharmaceutical ingredient-containing portion and one arm excludes the active pharmaceutical ingredient-containing portion. In some embodiments, all six arms comprise the active pharmaceutical ingredient-containing portion. In some embodiments, the first inert segment comprises PCL. In some embodiments, the first inert segment has a radial length of 0.1-5 mm. In some embodiments, the first inert segment has a radial length of 0.5-3 mm. In some embodiments, the first inert segment has a radial length of 1-1.5 mm. In some embodiments, the first disintegrating matrix segment comprises polycaprolactone. In some embodiments, the first disintegrating matrix segment further comprises poly(ethylene oxide). In some embodiments, the first disintegrating matrix segment further comprises DL-lactide/glycolide copolymer. In some embodiments, the first disintegrating matrix segment further comprises ferrosoferric oxide. In some embodiments, the first disintegrating matrix segment has a radial length of 0.1-5 mm. In some embodiments, the first disintegrating matrix segment has a radial length of 0.5-3 mm. In some embodiments, the first disintegrating matrix segment has a radial length of 0.7-1.3 mm. In some embodiments, the second inert segment comprises polycaprolactone. In some embodiments, the second inert segment further comprises bismuth subcarbonate. In some embodiments, the second inert segment has a radial length of 0.05-3 mm. In some embodiments, the second inert segment has a radial length of 0.1-1.5 mm. In some embodiments, the second inert segment has a radial length of 0.3-0.7 mm. In some embodiments, the second disintegrating matrix segment comprises polycaprolactone. In some embodiments, the second disintegrating matrix segment further comprises HPMCAS-MG. In some embodiments, the second disintegrating matrix segment further comprises poloxamer 407. In some embodiments, the second disintegrating matrix segment has a radial length of 0.1-5 mm. In some embodiments, the second disintegrating matrix segment has a radial length of 0.5-3 mm. In some embodiments, the second disintegrating matrix segment has a radial length of 1.5-2.5 mm. In some embodiments, the third inert segment comprises polycaprolactone. In some embodiments, the third inert segment further comprises bismuth subcarbonate. In some embodiments, the third inert segment has a radial length of 0.05-3 mm. In some embodiments, the third inert segment has a radial length of 0.1-1.5 mm. In some embodiments, the third inert segment has a radial length of 0.3-0.7 mm. In some embodiments, the fourth inert segment comprises polycaprolactone. In some embodiments, the fourth inert segment further comprises copovidone. In some embodiments, the fourth inert segment further comprises poloxamer 407. In some embodiments, the fourth inert segment further comprises a colorant. In some embodiments, the fourth inert segment has a radial length of 2-20 mm. In some embodiments, the fourth inert segment has a radial length of 4-15 mm. In some embodiments, the fourth inert segment has a radial length of 4.5-13.5 mm. In some embodiments, the fourth inert segment has a radial length of 12.1-13.3 mm. In some embodiments, the fourth inert segment has a radial length of 4.8-6 mm. In some embodiments, the active pharmaceutical ingredient-containing portion comprises risperidone or a pharmaceutically acceptable salt thereof. In some embodiments, the active pharmaceutical ingredient-containing portion further comprises polycaprolactone. In some embodiments, the active pharmaceutical ingredient-containing portion further comprises copovidone. In some embodiments, the active pharmaceutical ingredient-containing portion further comprises poloxamer 407. In some embodiments, the active pharmaceutical ingredient-containing portion further comprises vitamin E succinate. In some embodiments, the active pharmaceutical ingredient-containing portion further comprises silicon dioxide. In some embodiments, the active pharmaceutical ingredient-containing portion further comprises one or more colorants. In some embodiments, the active pharmaceutical ingredient-containing portion has a radial length of 3-12 mm. In some embodiments, the active pharmaceutical ingredient-containing portion has a radial length of 5-9 mm. In some embodiments, the active pharmaceutical ingredient-containing portion has a radial length of 6.5-8.5 mm. In some embodiments, the active pharmaceutical ingredient-containing portion comprises 10-80% by weight of an active pharmaceutical ingredient. In some embodiments, the active pharmaceutical ingredient-containing portion comprises 40-60% by weight of an active pharmaceutical ingredient. In some embodiments, the active pharmaceutical ingredient-containing portion comprises 20-40% by weight of an active pharmaceutical ingredient. In some embodiments, the at least one arm comprising an active pharmaceutical ingredient-containing portion comprises a coating. In some embodiments, the coating comprises polycaprolactone. In some embodiments, the coating further comprises copovidone. In some embodiments, the coating further comprises magnesium stearate. In some embodiments, the gastric residence system has a maximum diameter of 40-50 mm. In some embodiments, a respective portion of the flexible line connecting each arm is embedded in the fourth inert segment of each arm. In some embodiments, the flexible line is embedded in the distal end of each arm by heating the distal end, pressing a portion of the flexible line into the distal end, and cooling the distal end with the portion of the flexible line embedded. In some embodiments, heating the distal end comprises directing a laser at the distal end. In some embodiments, the laser comprises an infrared laser. In some embodiments, heating comprises convection heating. In some embodiments, the flexible line comprises an irregular cross-section. In some embodiments, the irregular cross-section comprises a braided cross-section. In some embodiments, the irregular cross-section comprises at least one of bumps, ridges, knots, barbs, or valleys. In some embodiments, each arm comprises a triangular cross-section. In some embodiments, the triangular cross-section has a base of 2.5-3.5 mm and a height of 2.1-3.1 mm. In some embodiments, the triangular cross-section has a base of 2.9-3.3 mm and a height of 2.4-3.1 mm. In some embodiments, the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, 100% poly-L-lactide, polyethylene terephthalate, polypropylene, polyamide, polyester, poly(ether ester), polytetrafluoroethylene, or polyvinylidinefluoride (PVDF). In some embodiments, the flexible line comprises one or more of poly(glycolic acid) (PGA), poly(lactic-co-glycolic acid) (PLGA), or polyethylene terephthalate (PET). In some embodiments, the flexible line has a diameter of 0.05-1 mm. In some embodiments, the flexible line has a cross-sectional area of 0.005-3.14 mm². In some embodiments, the flexible line has a cross-sectional area of 0.1-5 mm². In some embodiments, the flexible line has a cross-sectional area of 0.5-3.5 mm². In some embodiments, the flexible line has a cross-sectional area of 0.6-1 mm². In some embodiments, the flexible line has a surface roughness of 5-400 μm. In some embodiments, the flexible line has a surface roughness of 50-250 μm. In some embodiments, the flexible line is absorbable. In some embodiments, the flexible line is non-absorbable.

### BRIEF DESCRIPTION OF THE FIGURES

The invention will now be described, by way of example only, with reference to the accompanying drawings, in which:

FIGS. 1A-1C show various gastric residence system configurations, according to some embodiments.

FIG. 2 shows a gastric residence system comprising a plurality of arms and the bent geometry a gastric residence system can assume most easily when compressed by forces such as gastric contractions, according to some embodiments.

FIGS. 3A-3C show various methods by which a gastric residence system may pass through a pylorus prior to dissolving, according to some embodiments.

FIGS. 4A-4B show a gastric residence system with a flexible line and how the flexible line may help prevent premature passage through the pylorus, according to some embodiments.

FIGS. 5A-5B show two different configurations of gastric residence systems with flexible lines, according to some embodiments.

FIGS. 6A-6F show different braided configurations of flexible lines, according to some embodiments.

FIGS. 7A-7D show variations of welding pucks for laser welding of flexible lines to gastric residence systems, according to some embodiments.

FIG. 8 shows an exemplary gastric residence system with an embedded flexible line, according to some embodiments.

FIGS. 9A-9B show a two arm pull (TAPS) test of a gastric residence system with a flexible line, according to some embodiments.

FIGS. 10A-10B show a pull force test of a gastric residence system with a flexible line, according to some embodiments.

FIG. 11 shows flexible line adhesion of a flexible line to distal end portions of a gastric residence system comprising materials of various stiffness, according to some embodiments.

FIG. 12 shows flexible line adhesion of a flexible line to distal end portions of a gastric residence system having various cross-section thicknesses, according to some embodiments.

FIG. 13 shows flexible line adhesion of flexible lines with varying surface area and roughness to distal end portions of a gastric residence system, according to some embodiments.

FIG. 14 shows flexible line adhesion of single-stranded and braided flexible lines to distal end portions of a gastric residence system comprising various materials, according to some embodiments.

FIG. 15 shows the relationship between flexible line slippage and flexible line adhesion between various flexible line configurations and distal end portion materials, according to some embodiments.

FIG. 16 shows an exemplary gastric residence system with a flexible line, according to some embodiments.

FIG. 17 shows demographics of patients in a clinical study of a gastric residence system comprising risperidone, according to some embodiments.

FIG. 18 shows a mean pharmacokinetic profile of a gastric residence system comprising risperidone, according to some embodiments.

FIG. 19 shows PANSS scores over the course of a clinical study of a gastric residence system comprising risperidone, according to some embodiments.

FIG. 20 shows the incidence of gastrointestinal adverse events during a clinical study of a gastric residence system comprising risperidone, according to some embodiments.

FIG. 21 shows average SSS-8 scores over the course of a clinical study of a gastric residence system comprising risperidone, according to some embodiments.

### DETAILED DESCRIPTION

Described herein are flexible line configurations for gastric residence systems and methods of preparing gastric residence systems with a flexible line. As described above, gastric residence systems are designed to reside in the gastrointestinal tract of a patient for a predetermined residence time. After the residence time elapses, the gastric residence system breaks down into several pieces small enough to pass through the pylorus. However, if the gastric residence system bends or breaks into a configuration small enough to pass through the pylorus prematurely, the agent included in the gastric residence system is not administered to the patient as intended.

Accordingly, the gastric residence systems provided herein include a flexible line that connects each of the arms of a gastric residence system. The flexible line can help prevent the gastric residence system from prematurely passing through the pylorus.

Gastric residence systems are typically administered in a folded, closed, or collapsed configuration. When the gastric residence system enters the patient's stomach, it unfolds to assume an open configuration. The physical opening or unfolding of the gastric residence system results in a dosage form with an effective size that is too large to pass through the patient's pylorus. The deployed, or expanded, gastric residence system can stay in the patient's stomach for a predetermined period of time (e.g., 24 hours, 48 hours, 7 days, 10 days, etc.).

However, one challenge in particular with gastric residence systems is ensuring a consistent and accurate residence time. A gastric residence system that passes through the pylorus too early fails to administer the intended amount of agent, compromising the efficacy and reliability of the gastric residence system.

Accordingly, gastric residence systems provided herein are designed for more consistent and accurate residence times in a patient's stomach. In particular, gastric residence systems comprising a flexible line provided herein are more likely to resist premature passage through the pylorus. Thus, gastric residence systems provided herein are more likely to deliver consistent and accurate residence times, improving the efficacy and reliability of the gastric residence system.

### Definitions

As used herein, “gastric residence system” is a dosage form comprising an agent and is configured to be administered to a patient in a folded configuration. A “gastric residence dosage form” comprises a folded gastric residence system and is configured to hold the gastric residence system in a folded configuration until deployment. For example, a gastric residence dosage form may comprise a capsule and/or a capsule coating according to those described in U.S. Appln. No. 62/821,352 titled “Capsules and Capsule Coatings for Gastric Residence Dosage Forms” and/or U.S. Appln. No. 62/821,361 titled “Coatings for Gastric Residence Forms.”

A “carrier polymer” is a polymer suitable for blending with an agent, such as a drug, for use in the invention.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” (also referred to as a “tensile polymer”) is a polymer that is capable of being deformed by an applied force from its original shape for a period of time, and which then substantially returns to its original shape once the applied force is removed.

A “coupling polymer” is a polymer suitable for coupling any other polymers together, such as coupling a first carrier polymer-agent component to a second carrier polymer-agent component. Coupling polymers typically form the linker regions between other components.

A “time-dependent polymer” or “time-dependent coupling polymer” is a polymer that degrades in a time-dependent manner when a gastric residence system is deployed in the stomach. A time-dependent polymer is typically not affected by the normal pH variations in the stomach.

“Approximately constant plasma level” refers to a plasma level that remains within a factor of two of the average plasma level (that is, between 50% and 200% of the average plasma level) measured over the period that the gastric residence system is resident in the stomach.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “patient,” “individual,” or “subject” refers to a mammal, preferably a human or a domestic animal such as a dog or cat. In a most preferred embodiment, a patient, individual, or subject is a human.

The “diameter” of a particle as used herein refers to the longest dimension of a particle.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods of the invention can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

A “flexural modulus” of a material is an intrinsic property of a material computed as the ratio of stress to strain in flexural deformation of the material as measured by a 3-point bending test. The flexural modulus of various components of the gastric residence systems disclosed herein may be measured. For example, segments of the arms of a gastric residence system (e.g., inert segments, linkers such as time-dependent disintegrating matrix segments and enteric disintegrating matrix segments, etc.) may be characterized by their respective flexural moduli. Although the segments are described herein as being components of the gastric residence system, the flexural modulus of the material comprising the segments may be measured in isolation. For example, a segment of a gastric residence system may be too short to measure the flexural modulus, but a longer sample of the same material may be used to accurately determine the flexural modulus. The longer sample used to measure the flexural modulus should have the same cross-sectional dimensions (shape and size) as the segment used in the gastric residence system. The flexural modulus is measured using a 3-point bending test based on the ASTM standard 3-point bending test (ASTM D790) using a 10 mm distance between supports. The 3-point bending test modifies the ASTM standard to accommodate shorter material samples and materials with non-rectangular cross-sections (e.g., triangular cross-sections). A first set of material samples are incubated at 37° C. The samples are removed from the media, dried, and measured. A second set of material samples are not incubated before testing. Both sets of samples are submerged in a 37±1° C. deionized water bath and oriented such that the longest line of symmetry for the cross section of the sample is positioned vertically, and the flexural modulus is measured by applying force downward. If the longest line of symmetry for the cross section of the sample is perpendicular to a single flat edge, the single flat edge should be positioned upward. If the cross-section of the sample is triangular, the apex of the triangle should be faced downward. As force is applied downward, force and displacement are measured, and the slope at the linear region is obtained to calculate the flexural modulus and flexural strength.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present invention.

In addition to the embodiments and methods disclosed here, additional embodiments of gastric residence systems, and methods of making and using such systems, are disclosed in International Patent Application Nos. WO 2015/191920, WO 2015/191925, WO 2017/070612, WO 2017/100367, WO 2017/205844, and WO 2018/227147, which are incorporated by reference herein in their entirety.

### General Principles of Gastric Residence Systems

Provided herein are flexible line configurations for gastric residence systems. Gastric residence systems are designed to be administered to a stomach of a patient, either by swallowing or other method of administration (e.g., feeding tube or gastric tube). Once a gastric residence system is in place in the stomach, the system remains in the stomach for the desired residence period (e.g., three days, seven days, two weeks, etc.). During the residence period, the system resists passage through the pylorus, which separates the stomach and the small intestine. The system releases an agent (e.g., an active pharmaceutical ingredient or drug) into the stomach over the residence period at a controlled rate of release. While residing in the stomach, the system may not interfere with the normal passage of food or other gastric contents. Once the desired residence period has elapsed, the system may pass through the pylorus and be eliminated from the patient. If the system prematurely passes from the stomach into the small intestine, it does not cause intestinal obstruction, and again is readily eliminated from the patient.

To administer a gastric residence system to a patient, the gastric residence system may be folded into a configuration small enough to be swallowed or otherwise administered. In some embodiments, the folded gastric residence system is retained in a capsule or other container which can be swallowed by the patient or otherwise administered. In some embodiments, a capsule may comprise at least one of gelatin, hydroxypropyl methylcellulose, or pullulan.

In some embodiments, the folded gastric residence system may also be secured by a dissolvable retaining band or sleeve that can prevent premature deployment of the gastric residence system in case of a failure of the capsule or other container. A gastric residence system folded and retained in a folded configuration with a sleeve or band may be encapsulated by a capsule. In some embodiments, a sleeve or band may comprise at least one of gelatin, hydroxypropyl methylcellulose, or pullulan.

Once the capsule or other container storing the gastric residence system reaches the stomach of a patient, the capsule or container dissolve and release the folded gastric retention system. Upon release, the gastric retention system may unfold and assume an open configuration. The open configuration may be a stellate shape. The dimensions of the gastric residence system in the open configuration are, when left unaltered, suitable to prevent passage of the gastric residence system through the pylorus for the desired residence period.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pylorus into the duodenum.

The gastric residence system releases an agent (e.g., while in the stomach). The gastric residence system may comprise a plurality of polymer-agent components. In one embodiment, the polymer-agent components comprise a carrier polymer, a dispersant, and an agent (or a salt thereof). In another embodiment, the polymer-agent components comprise a carrier polymer and an agent (or a salt thereof). The plurality of polymer-agent components are linked together by one or more elastomer components and/or one or more coupling polymer components. Agent is eluted from the carrier polymer-agent components into the gastric fluid of the patient over the desired residence time of the system. Release of the agent is controlled by appropriate formulation of the carrier polymer-agent components, including by the use of the dispersant in formulation of the carrier polymer-agent components, and by milling of the agent to particles of desired size prior to blending the agent with the carrier polymer and dispersant. In addition, coatings can be applied to outer surfaces of the gastric residence system. The coatings may include additional agents or agents that can affect the release of agents or the residence period of the gastric residence system.

Once the desired residence time has elapsed, the gastric residence system passes out of the stomach. To do so, various components of the gastric residence system are designed to weaken and degrade. The specific dimensions of the system are also taken into consideration. In its intact, open configuration, the gastric residence system is designed to resist passage through the pylorus. However, coupling polymer components of the gastric residence system are chosen such that they gradually degrade over the specified residence period in the stomach. When the coupling polymer components are sufficiently weakened by degradation, the gastric residence system loses critical resilience to compression or size reduction and can break apart into smaller pieces. The reduced-size system and any smaller pieces are designed to pass through the pylorus. The system then passes through the intestines and is eliminated from the patient. In some embodiments, a gastric residence system may be designed to weaken at specific locations such that the gastric residence system can pass through a pyloric valve intact once the residence time expires without degrading into numerous smaller pieces.

### Overall System Configuration

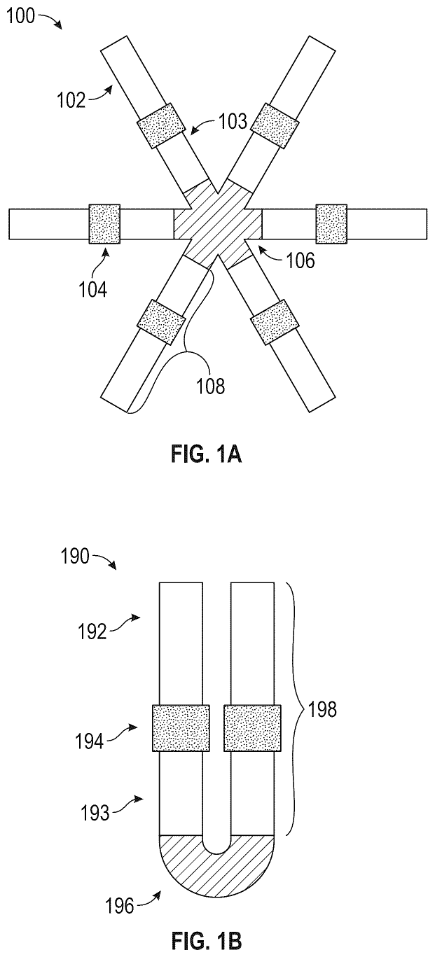

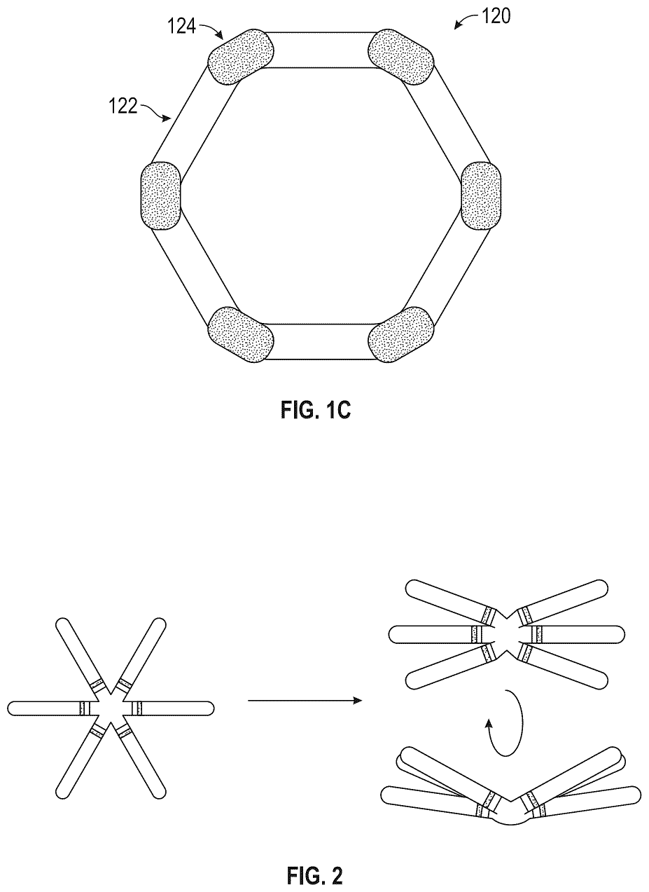

Gastric residence systems may be prepared in different configurations. The “stellate” configuration of a gastric residence system is also known as a “star” (or “asterisk”) configuration. An example of a stellate system is shown schematically in FIG. 1A. An example of a stellate system 100 is shown schematically in FIG. 1A. Multiple arms (only one of which, 108, is labeled for the sake of brevity), are affixed to disk-shaped central elastomer 106. The arms depicted in FIG. 1A are comprised of segments 102 and 103, joined by a coupling polymer or linker region 104 (only one of which is labeled for the sake of brevity) which serves as a linker region. This configuration permits the system to be folded or compacted at the central elastomer.

FIG. 1B shows a folded configuration 190 of the gastric residence system of FIG. 1A. Only two arms are illustrated in FIG. 1B for the sake of brevity. Segments 192 and 193, linker region 194, elastomer 196, and arm 198 of FIG. 1B correspond to segments 102 and 103, linker region 104, elastomer 106, and arm 108 of FIG. 1A, respectively. When folded, the overall length of the system is reduced by approximately a factor of two, and the system can be conveniently placed in a container such as a capsule or other container suitable for oral administration. When the capsule reaches the stomach, the capsule dissolves, releasing the gastric residence system. The gastric residence system then unfolds into its uncompacted state, which is retained in the stomach for the desired residence period.

While the linker regions 104 are shown as slightly larger in diameter than the segments 102 and 103 in FIG. 1A, they can be the same diameter as the segments, so that the entire arm 102-104-103 has a smooth outer surface.

In some embodiments, the stellate system may have an arm composed of only one segment, which is attached to the central elastomer by a linker region. This corresponds to FIG. 1A with the segments 103 omitted. The single-segment arms comprising segments 102 are then directly attached to central elastomer 106 via the linkers 104. The linkers can comprise a coupling polymer or a disintegrating matrix.

A stellate system can be described as a gastric residence system for administration to the stomach of a patient, comprising an elastomer component, and a plurality of at least three carrier polymer-agent components comprising a carrier polymer and an agent or a salt thereof, attached to the elastomer component, wherein each of the plurality of carrier polymer-agent components is an arm comprising a proximal end, a distal end, and an outer surface therebetween; wherein the proximal end of each arm is attached to the elastomer component and projects radially from the elastomer component, each arm having its distal end not attached to the elastomer component and located at a larger radial distance from the elastomer component than the proximal end; wherein each arm independently comprises one or more segments, each segment comprising a proximal end, a distal end, and an outer surface therebetween. In some embodiments, when two or more segments are present in an arm, each segment is attached to an adjacent segment via a linker region. In some embodiments, when two or more segments are present in an arm, one segment is directly attached to the other segment, without using a linker region. The linker region can be a coupling polymer or a disintegrating matrix. The arms can be attached to the central elastomer via a coupling polymer or a disintegrating matrix, and can have intervening portions of interfacing polymers. For the plurality of at least three arms, or for a plurality of arms, a preferred number of arms is six, but three, four, five, seven, eight, nine, or ten arms can be used. The arms should be equally spaced around the central elastomer; if there are N arms, there will be an angle of about 360/N degrees between neighboring arms.

FIG. 1C shows another possible overall configuration 120 for a gastric residence system, which is a ring configuration. Segments 122 are joined by coupling polymer or linker region 124 (only one segment and one coupling linkage are labeled for clarity). The coupling polymer/linker region in this design must also function as an elastomer, to enable the ring to be twisted into a compacted state for placement in a container, such as a capsule.

In one embodiment of the stellate configuration, the segments 102 and 103 comprise a carrier polymer blended with an agent or drug. In one embodiment of the ring configuration, the segments 122 comprise a carrier polymer blended with an agent or drug.

The coupling polymers of the gastric residence system, which serve as linker regions, are designed to break down gradually in a controlled manner during the residence period of the system in the stomach. If the gastric residence system passes prematurely into the small intestine in an intact form, the system is designed to break down much more rapidly to avoid intestinal obstruction. This is readily accomplished by using enteric polymers as coupling polymers. Enteric polymers are relatively resistant to the acidic pH levels encountered in the stomach, but dissolve rapidly at the higher pH levels found in the duodenum. Use of enteric coupling polymers as safety elements protects against undesired passage of the intact gastric residence system into the small intestine. The use of enteric coupling polymers also provides a manner of removing the gastric residence system prior to its designed residence time; should the system need to be removed, the patient can drink a mildly alkaline solution, such as a sodium bicarbonate solution, or take an antacid preparation such as hydrated magnesium hydroxide (milk of magnesia) or calcium carbonate, which will raise the pH level in the stomach and cause rapid degradation of the enteric coupling polymers. The gastric residence system will then break apart and be eliminated from the patient. In the system shown in FIG. 1A, at least the coupling polymer used for the couplings 104 are made from such enteric polymers.

In additional embodiments, a time-dependent coupling polymer or linker can be used. Such a time-dependent coupling polymer or linker degrades in a predictable, time-dependent manner. In some embodiments, the degradation of the time-dependent coupling polymer or linker may not be affected by the varying pH of the gastrointestinal system.

In additional embodiments, different types of linkers can be used in the gastric residence systems. That is, both enteric linkers (or enteric coupling polymers) and time-dependent linkers (or time-dependent coupling polymers) can be used. In some embodiments, a single multi-segment arm of a stellate system can use both an enteric linker at some linker regions between segments, and a time-dependent linker at other linker regions between segments.

Linker regions are typically about 100 microns to about 2 millimeter in width, such as about 200 um to about 2000 um, about 300 um to about 2000 um, about 400 um to about 2000 um, about 500 um to about 2000 um, about 600 um to about 2000 um, about 700 um to about 2000 um, about 800 um to about 2000 um, about 900 um to about 2000 um, about 1000 um to about 2000 um, about 1100 um to about 2000 um, about 1200 um to about 2000 um, about 1300 um to about 2000 um, about 1400 um to about 2000 um, about 1500 um to about 2000 um, about 1600 um to about 2000 um, about 1700 um to about 2000 um, about 1800 um to about 2000 um, or about 1900 um to about 2000 um; or about 100 um to about 1900 um, about 100 um to about 1800 um, about 100 um to about 1700 um, about 100 um to about 1600 um, about 100 um to about 1500 um, about 100 um to about 1400 um, about 100 to about 1300 um, about 100 um to about 1200 um, about 100 um to about 1100 um, about 100 um to about 1000 um, about 100 um to about 900 um, about 100 um to about 800 um, about 100 um to about 700 um, about 100 um to about 600 um, about 100 um to about 500 um, about 100 um to about 400 um, about 100 um to about 300 um, or about 100 um to about 200 um. Linker regions can be about 100 um, about 200 um, about 300 um, about 400 um, about 500 um, about 600 um, about 700 um, about 800 um, about 900 um, about 1000 um, about 1100 um, about 1200 um, about 1300 um, about 1400 um, about 1500 um, about 1600 um, about 1700 um, about 1800 um, about 1900 um, or about 2000 um in width, where each value can be plus or minus 50 um (±50 um).

The central elastomeric polymer of a stellate system is typically not an enteric polymer; however, the central elastomeric polymer can also be made from such an enteric polymer where desirable and practical.

The central elastomer should have a specific durometer and compression set. The durometer is important because it determines the folding force of the dosage form and whether it will remain in the stomach; in an embodiment, the range is from about 60 to about 90A. The compression set should be as low as possible to avoid having permanent deformation of the gastric residence system when stored in the capsule in its compacted configuration. In one embodiment, the range is about 10% to about 20%. Non-limiting examples of materials that fit these requirements are the QP1 range of liquid silicone rubbers from Dow Corning. In any embodiment with a central elastomer, the QP1-270 (70A durometer) liquid silicone rubber can be used. In some embodiments, the central elastomer may comprise a 40A, 50A, or 60A durometer liquid silicone rubber (Shin Etsu).

Segments and arms of the gastric residence systems can have cross-sections in the shape of a circle (in which case the segments are cylindrical), a polygon (such as segments with a triangular cross-section, rectangular cross-section, or square cross-section), or a pie-shaped cross-section (in which case the segments are cylindrical sections). Segments with polygon-shaped or pie-shaped cross-sections, and ends of cylindrically-shaped sections which will come into contact with gastric tissue, can have their edges rounded off to provide rounded corners and edges, for enhanced safety in vivo. That is, an arc can be used to transition from one edge or plane to another edge or plane. Thus, a “triangular cross-section” includes cross-sections with an approximately triangular shape, such as a triangle with rounded corners. An arm with a triangular cross-section includes an arm where the edges are rounded, and the corners at the end of the arm are rounded. Rounded corners and edges are also referred to as fillet corners, filleted corners, fillet edges, or filleted edges.

However, it has been demonstrated that gastric residence systems of a stellate shape can bend into a configuration that allows for premature passage through the pylorus of a patient. Gastric residence systems that prematurely pass through the pylorus fail to deliver the agent of the gastric residence system to the patient. Further, premature passage causes inconsistency, causes unreliability, and compromises the efficacy of the gastric residence system.

FIG. 2 shows a stellate-shaped gastric residence system having a plurality of arms. One example of a bended configuration is shown on the right side of FIG. 2. Due to forces in the stomach (e.g., peristaltic forces), gastric residence systems may bend into configurations, such as that shown in FIG. 2, that can allow for premature passage through the pylorus.

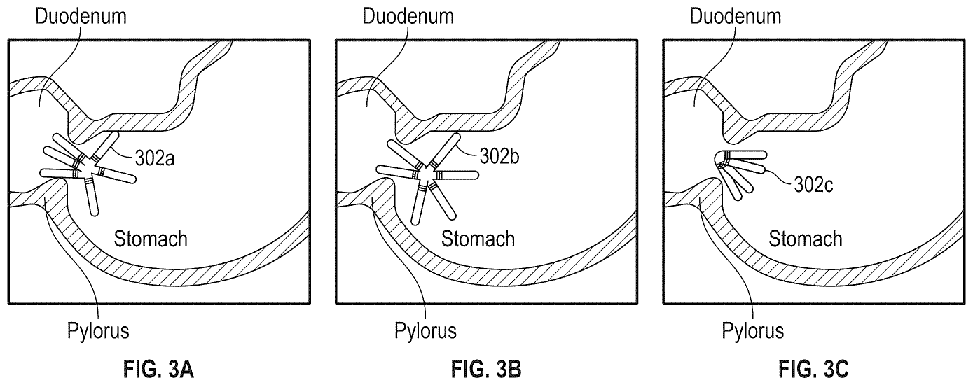

Other possible bended configurations are shown in FIGS. 3A-3C. Specifically, FIGS. 3A-3C show three different configurations that a gastric residence system may assume that can allow for premature passage through the pylorus. As shown in each Figure, the relatively stiff arms of the gastric residence system remain straight. However, because the core of each of the gastric residence systems has a higher flexibility than the arms, the core can bend. The bending of the core can allow gastric residence systems having relatively stiff arms to prematurely pass through the pylorus of a patient.

As shown in FIG. 3A, gastric residence system 302a is shown in a bended configuration having three arms leading through the pyloric opening. FIG. 3B shows gastric residence system 302b in a bended configuration having two arms leading through the pyloric opening. FIG. 3C shows gastric residence system 302c in a bended configuration analogous to the shape of a shuttlecock and having the core leading through the pyloric opening.

Accordingly, described herein are gastric residence systems comprising a flexible line. A flexible line connecting the distal tips of adjacent arms of a gastric residence system can help prevent premature passage of the gastric residence system through a patient's pylorus. Flexible line configurations and gastric residence systems comprising flexible lines are described in more detail with respect to the arms and coupling polymers of a gastric residence system.

### Flexible Line Configurations

As described, including a flexible line in the gastric residence system may help prevent the gastric residence system from prematurely passing through a patient's pylorus. Accordingly, the flexible line configurations described herein can help improve the efficacy and reliability of gastric residence systems.

As used herein, a “flexible line” may refer to a strand, fiber, suture, or filament, or a bundle of strands, fibers, sutures, or filaments, connecting two or more parts of a gastric residence system. In some embodiments, a flexible line can have a circular, flat, or irregular cross-section.

A flexible line may be attached to a distal end of each arm of a gastric residence system. In some embodiments, the flexible line may be embedded in the distal end of each arm, such that the flexible line connects the distal end of each arm to the distal ends of the adjacent arms. The flexible line may prevent the gastric residence system from prematurely passing through the pylorus by maintaining the spacing of the arms in the expanded configuration, thereby increasing the force required to compress the gastric residence system into a configuration small enough to prematurely pass through the pylorus. The flexible line may also prevent one or more arms from prematurely entering the pylorus.

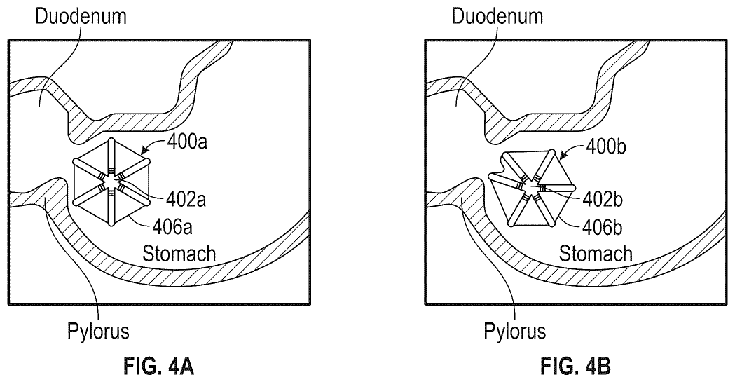

FIGS. 4A and 4B show how the inclusion of a flexible line affects the most common bending and passage modes of an intact gastric residence system through the pylorus. For example, gastric residence system 400a of FIG. 4A comprises a central core 402a and a plurality of arms. As shown, each arm 404a of the plurality of arms extends radially from the central core 402. Each arm 404 is attached to core 402a at a proximal end. Flexible line 406a is shown attached to the distal end of each arm 404a. FIG. 4A shows gastric residence system 400a in an open configuration. As shown, when gastric residence system 400a remains in an open configuration, flexible line 406a helps prevent gastric residence system 400a from passing prematurely through a pylorus.

FIG. 4B shows gastric residence system 400b in a bended configuration. Gastric residence system 400b comprises core 402b, arms 404b, and flexible line 406b. As shown, even if gastric residence system 400b bends into a configuration that might otherwise allow for premature passage through a patient's pylorus (see FIG. 3B), flexible line 408b can help prevent the device from passing. In particular, flexible line 408b is flexible and stretchable such that it can maintain its integrity despite gastric forces that may bend and contort gastric residence system 400b.

In some embodiments, one or more arms of the gastric residence system may comprise a separate tip portion located at a distal end of the one or more arms. The flexible line may be connected to the arm by way of the separate tip portion at the distal end. In some embodiments, a separate tip portion is affixed to the end of an arm having an active pharmaceutical ingredient-containing portion, such that the flexible line can connect to the separate tip portion rather than the active pharmaceutical ingredient-containing portion. In some embodiments, the flexible line may connect directly to the active pharmaceutical ingredient-containing portion.

The separate tip portion may also be configured to separate from the rest of the arm when in a gastric environment. In particular, the separate tip portion may be configured to separate from the arm, allowing the flexible line to also separate from the gastric residence system. This separation may be fine-tuned such that the separate tip portion and flexible line separate once a predetermined gastric residence time approaches expiration, such that the gastric residence system separates and passes through a patient's pylorus at the expiration of the predetermined gastric residence time. If the separate tip portion and/or flexible line separate too early, the gastric residence system risks passing through the patient's pylorus prematurely.

In some embodiments, the separate tip portion may comprise the same material as the distal ends of the non-drug eluting arms of the gastric residence system. In some embodiments, the separate tip portion may comprise a different material than the distal ends of the non-drug eluting arms of the gastric residence system. The separate tip portion may comprise one or more polymers (including enteric polymers and/or pH-independent polymers), enteric materials, plasticizers, acids, and/or colorants.

In some embodiments, the separate tip portion and/or the distal ends may comprise one or more polymers. Suitable polymers may include polycaprolactone (PCL), polyvinylpyrrolidone (copovidone), polyoxyethylene-polyoxypropylene (Poloxamer 407, P407), thermoplastic polyurethanes (e.g., Pathway™ by Lubrizol), poly-D-lysine (PDL), poly(L-lactide), poly(L-lactide-co-D,L lactide), polyglycolide, poly(L-lactide-co-glycolide), poly(L-lactide co-caprolactone), poly(DL-lactide), poly(D,L-lactide-co-glycolide), polyethylene oxide, poly(lactic-co-glycolic acid), poly(lactic acid), polydioxanone, cellulose, hydroxypropyl methylcellulose acetate succinate MG grade (HPMCAS-MG), or combinations thereof. A preferred polymer is PCL. In some embodiments, the composition of a separate tip portion and/or distal end may be the same as the composition of a linker component (e.g., an enteric disintegrating matrix segment or a time-dependent disintegrating matrix segment). In some embodiments, the composition of a separate tip portion and/or distal end may be different than the composition of a linker component. In some embodiments, a separate tip portion and/or distal end may comprise from 10 to 50 wt. % polymer or 10 to 100 wt. % polymer. In some embodiments, a separate tip portion and/or distal end may comprise less than 100 wt. %, less than 90 wt. %, less than 80 wt. %, less than 70 wt. %, less than 60 wt. %, less than 50 wt. %, less than 40 wt. %, less than 30 wt. %, or less than 20 wt. % polymer. In some embodiments, a separate tip portion and/or distal end may comprise more than 10 wt. %, more than 20 wt. %, more than 30 wt. %, more than 40 wt. %, more than 50 wt. %, more than 60 wt. %, more than 70 wt. %, more than 80 wt. %, or more than 90 wt. % polymer. In some embodiments, a separate tip portion and/or distal end may comprise 100% polymer, such as 100% PCL. In some embodiments, the separate tip portion and/or distal end may comprise PCL and copovidone.

In some embodiments, the separate tip portion and/or the distal ends may further comprise an enteric material. The enteric material may comprise an enteric polymer. For example, suitable enteric polymers include Cellulose acetate phthalate, Hydroxypropyl methylcellulose phthalate 50, Hydroxypropyl methylcellulose phthalate 55, Polyvinylacetate phthalate, Methacrylic acid-methyl methacrylate copolymer (1:1), Methacrylic acid-methyl methacrylate copolymer (2:1), Methacrylic acid-ethyl acrylate copolymer (2:1), Shellac, Hydroxypropyl methylcellulose acetate succinate, Poly (methyl vinyl ether/maleic acid) monoethyl ester, or Poly (methyl vinyl ether/maleic acid) n-butyl ester. In some embodiments, the separate tip portion and/or the distal ends may comprise from 20 to 90 wt. % enteric material. In some embodiments, the separate tip portion and/or the distal ends may comprise less than 90 wt. %, less than 80 wt. %, less than 70 wt. %, less than 60 wt. %, less than 50 wt. %, less than 40 wt. %, or less than 30 wt. % enteric material. In some embodiments, the separate tip portion and/or the distal ends may comprise more than 20 wt. %, more than 30 wt. %, more than 40 wt. %, more than 50 wt. %, more than 60 wt. %, more than 70 wt. %, or more than 90 wt. % enteric material.

In some embodiments, the separate tip portion and/or the distal ends may further comprise a plasticizer. Suitable plasticizers may include propylene glycol, P407, triethyl citrate, triacetin, dibutyl sebacate, and/or polyethylene glycol. In some embodiments, the separate tip portion and/or the distal ends may comprise from 1 to 20 wt. % plasticizer. In some embodiments, the separate tip portion and/or the distal ends may comprise less than 20 wt. %, less than 15 wt. %, less than 10 wt. %, or less than 5 wt. % plasticizer. In some embodiments, the separate tip portion and/or the distal ends may comprise more than 1 wt. %, more than 5 wt. %, more than 10 wt. %, or more than 15 wt. % plasticizer.

In some embodiments, the separate tip portion and/or the distal ends may further comprise an acid. Suitable acids can include stearic acid or other fatty acids. In some embodiments, the separate tip portion and/or the distal ends may comprise from 1 to 20 wt. % or from 1 to 10 wt. % acid. In some embodiments, the separate tip portion and/or the distal ends may comprise less than 20 wt. %, less than 15 wt. %, less than 10 wt. %, or less than 5 wt. % acid. In some embodiments, the separate tip portion and/or the distal ends may comprise more than 1 wt. %, more than 5 wt. %, more than 10 wt. %, or more than 15 wt. % acid.

In some embodiments, the separate tip portion and/or the distal ends may further comprise one or more colorants. A colorant may be a dye, a pigment, or a lake (e.g., FD&C Blue No. 1 Aluminum Lake). Alternatively or in addition, the separate tip portion and/or the distal ends may further comprise a radiopaque agent, such as bismuth subcarbonate and/or barium sulfate.

In some embodiments, the composition of the separate tip portion and/or the distal ends may be selected to improve the adhesive force between the separate tip portion and/or the distal ends and the flexible line (referred to herein as flexible line adhesion). Flexible line adhesion is the force required to separate a flexible line from the arm to which it is attached. Improving flexible line adhesion can improve gastric retention of a gastric residence system. If the flexible line is strongly adhered to an arm, the risk of flexible line slippage is reduced. Slippage refers to the release of the flexible line from one or more arms of the gastric residence system. When the flexible line slips, one or more arms of the gastric residence system may move from their original positions, which may allow the gastric residence system to fold into a configuration that allows it to prematurely exit the stomach through the pylorus. Thus, improving flexible line adhesion to the separate tip portions of the arms of the gastric residence system may reduce the risk of premature passage of the gastric residence system. In some embodiments, flexible line adhesion may be measured using the TAPS method described herein.

In some embodiments, the flexible line adhesion between an arm and the flexible line may be about 5-25 N or about 8-25 N. In some embodiments, the flexible line adhesion may be at least 5 N, at least 6 N, at least 7 N, at least 8N, at least 9 N, at least 10 N, at least 15 N, or at least 20 N. In some embodiments, the flexible line adhesion may be less than 25 N, less than 20 N, less than 15 N, less than 10 N, less than 9 N, less than 8 N, less than 7 N, or less than 6 N.

In some embodiments, the stiffness of the separate tip portion and/or the distal ends may affect flexible line adhesion. In some embodiments, increasing the stiffness of the separate tip portion and/or the distal ends may improve flexible line adhesion. Stiffness of the separate tip portion and/or the distal ends may be quantified by their flexural modulus. Flexural modulus may be measured using a 3-point bending test (e.g., the modified 3-point bending test described herein). In some embodiments, suitable materials may have a flexural modulus of about 50 MPa to about 5 GPa or about 50 MPa to about 1 GPa. In some embodiments, suitable materials may have a flexural modulus of at least about 50 MPa, at least about 150 MPa, at least about 250 MPa, at least about 350 MPa, at least about 450 MPa, at least about 550 MPa, at least about 650 MPa, at least about 750 MPa, at least about 850 MPa, at least about 950 MPa, at least about 1 GPa, at least about 2 GPa, at least about 3 GPa, or at least about 4 GPa. In some embodiments, suitable materials may have a flexural modulus of less than about 5 GPa, less than about 4 GPa, less than about 3 GPa, less than about 2 GPa, less than about 1 GPa, less than about 900 MPa, less than about 800 MPa, less than about 700 MPa, less than about 600 MPa, less than about 500 MPa, less than about 400 MPa, less than about 300 MPa, less than about 200 MPa, or less than about 100 MPa. In some embodiments, suitable materials may have a flexural modulus of about 50 MPa to about 500 MPa, about 150 MPa to about 600 MPa, about 250 MPa to about 700 MPa, about 350 MPa to about 800 MPa, about 450 to about 900 MPa, about 550 MPa to about 1 GPa, about 650 MPa to about 1 GPa, about 750 MPa to about 1 GPa, about 850 MPa to about 1 GPa, about 950 MPa to about 1 GPa, about 1 GPa to about 3 GPa, or about 3 GPa to about 5 GPa.

In some embodiments, the stiffness of the separate tip portion and/or the distal ends may be quantified by their flexural strength. Flexural strength may be measured using a 3-point bending test (e.g., the modified 3-point bending test described herein). In some embodiments, the flexural strength of the separate tip portions and/or distal ends may be about 5 to about 100 N. In some embodiments, the flexural strength may be at least about 5 N, at least about 8 N, at least about 10 N, at least about 20 N, at least about 30 N, at least about 40 N, at least about 50 N, at least about 60 N, at least about 70 N, at least about 80 N, or at least about 90 N. In some embodiments, the flexural strength may be less than about 100 N, less than about 90 N, less than about 80 N, less than about 70 N, less than about 60 N, less than about 50 N, less than about 40 N, less than about 30 N, less than about 20 N, less than about 10 N, or less than about 8 N. In some embodiments, the flexural strength may be about 5 N to about 10 N, about 10 N to about 20 N, about 20 N to about 40 N, about 30 N to about 50 N, about 40 N to about 60 N, about 50 N to about 70 N, about 60 N to about 80 N, about 70 N to about 80 N, about 80 N to about 90 N, or about 90 N to about 100 N.

In some embodiments, the stiffness of the separate tip portion and/or the distal ends may be quantified by their maximum force. Maximum force may be measured using a 3-point bending test (e.g., the modified 3-point bending test described herein) and may represent the maximum value on a force-displacement curve generated from performing the 3-point bending test. In some embodiments, the maximum force of the separate tip portion and/or the distal ends may be about 3 to about 100 N or about 5 to about 80 N. In some embodiments, the maximum force may be at least about 3 N, at least about 5 N, at least about 15 N, at least about 25 N, at least about 35 N, at least about 45 N, at least about 55 N, at least about 65 N, at least about 75 N, at least about 85 N, or at least about 95 N. In some embodiments, the maximum force may be less than about 100 N, less than about 90 N, less than about 80 N, less than about 70 N, less than about 60 N, less than about 50 N, less than about 40 N, less than about 30 N, less than about 20 N, less than about 10 N, or less than about 5 N. In some embodiments, the maximum force may be about 3 N to about 20 N, about 5 N to about 30 N, about 20 N to about 40 N, about 30 N to about 50 N, about 40 N to about 60 N, about 50 N to about 70 N, about 60 N to about 80 N, or about 70 N to about 80 N.

In some embodiments, the stiffness of the separate tip portion and/or the distal ends, as measured by the flexural modulus, flexural strength, and/or maximum force, may be stable over time in a gastric environment. In some embodiments, the stiffness of the separate tip portion and/or the distal end material when incubated in fasted state simulated gastric fluid (FaSSGF) for 1 day may be within about 10% of the stiffness of the separate tip portion and/or the distal end material when incubated in FaSSGF for 14 days. In some embodiments, the stiffness of the separate tip portion and/or distal end material may change less than 20%, less than 15%, less than 10%, less than 8%, less than 5%, or less than 3% from day 1 to day 14 in FaSSGF. In some embodiments, the stiffness of the separate tip portion and/or distal end material may change at least 1%, at least 3%, at least 5%, at least 8%, at least 10%, or at least 15% from day 1 to day 14 in FaSSGF.

In some embodiments, the separate tip portion and/or the distal ends may optionally be radiopaque, such that they can be located via abdominal X-ray if necessary. In some embodiments, one or more of the materials used to construct the separate tip portions is sufficiently radiopaque for X-ray visualization. In other embodiments, a radiopaque substance is added to one or more materials of the separate tip portion and/or the distal ends, coated onto one or more materials of the separate tip portion and/or the distal ends, or added to a small portion of the separate tip portion and/or the distal ends. Examples of suitable radiopaque substances are barium sulfate, bismuth subcarbonate, bismuth oxychloride, and bismuth trioxide. For example, the one or more polymers of the separate tip portion and/or the distal ends may include radiopaque PCL e.g., 90% PCL with 10% bismuth subcarbonate (rPCL10), 95% PCL with 5% bismuth subcarbonate (rPCL5), or 70% PCL with 30% bismuth subcarbonate (rPCL30).

In some embodiments, the arms of the gastric residence system (and thus the separate tip portion and/or the distal ends of the arms of the gastric residence system) may have cross-sections in the shape of a circle, a polygon (e.g., a triangle, a rectangle, or a square), or a pie-shape. A triangular cross-section may be preferred. In some embodiments, segments with polygon-shaped or pie-shaped cross-sections may have their sharp edges rounded off to provide rounded corners and edges, for enhanced safety in vivo. That is, instead of having a sharp transition between intersecting edges or planes, an arc is used to transition from one edge or plane to another edge or plane. Thus, a triangular cross-section includes cross-sections with an approximately triangular shape, such as a triangle with rounded corners. Rounded corners and edges are referred to as fillet corners, filleted corners, fillet edges, or filleted edges.

In some embodiments, the arms of the gastric residence system (and thus the separate tip portion and/or the distal ends of the arms of the gastric residence system) may have triangular cross-sections. The triangular cross-section may be in the shape of an equilateral triangle. The size of the cross-section may be selected such that the gastric residence system and the flexible line, when folded, can fit inside of a capsule for delivery to a patient. In some embodiments, each leg of the equilateral triangle may be about 2.5 to about 3.5 mm in length. In some embodiments, each leg of the equilateral triangle may be at least about 2.5 mm in length, at least about 2.6 mm in length, at least about 2.7 mm in length, at least about 2.8 mm in length, at least about 2.9 mm in length, at least about 3.0 mm in length, at least about 3.1 mm in length, at least about 3.2 mm in length, at least about 3.3 mm in length, or at least about 3.4 mm in length. In some embodiments, each leg of the equilateral triangle may be less than about 3.5 mm in length, less than about 3.4 mm in length, less than about 3.3 mm in length, less than about 3.2 mm in length, less than about 3.1 mm in length, less than about 3.0 mm in length, less than about 2.9 mm in length, less than about 2.8 mm in length, less than about 2.7 mm in length, or less than about 2.6 mm in length. In some embodiments, each leg of the equilateral triangle may be about 2.5 mm in length to 3.4 mm in length, about 2.6 mm in length to 3.5 mm in length, about 2.6 mm in length to about 3.4 mm in length, about 2.7 mm in length to about 3.4 mm in length, about 2.6 mm in length to about 3.3 mm in length, about 2.7 mm in length to about 3.3 mm in length, about 2.7 mm in length to about 3.2 mm in length, about 2.8 mm in length to about 3.3 mm in length, about 2.8 mm in length to about 3.2 mm in length, about 2.8 mm in length to about 3.1 mm in length, about 2.9 mm in length to about 3.2 mm in length, about 3.0 mm in length to about 3.2 mm in length, or about 2.9 mm in length to 3.1 mm in length.

In some embodiments, the separate tip portion and/or the distal ends of the arms of the gastric residence system may soften upon hydration. In some embodiments, the separate tip portion and/or the distal ends may soften about 10 to about 80% upon hydration in a gastric environment. In some embodiments, the separate tip potion and/or the distal ends may not soften upon hydration in a gastric environment (i.e., may soften 0%). In some embodiments, the separate tip portion and/or the distal ends may stiffen upon hydration in a gastric environment. In some embodiments, the separate tip portion and/or the distal ends may soften at least about 10%, at least about 20%, at least about 30%, at least about 40%, at least about 50%, at least about 60%, or at least about 70% upon hydration in a gastric environment. In some embodiments, the separate tip portion and/or the distal ends may soften less than about 80%, less than about 70%, less than about 60%, less than about 50%, less than about 40%, less than about 30%, or less than about 20% upon hydration in a gastric environment. In some embodiments, the separate tip portion and/or the distal ends may soften at least about 10% to about 70%, at least about 20% to about 70%, at least about 30% to about 70%, at least about 40% to about 70%, at least about 50% to about 70%, at least about 60% to about 70%, about 10% to 60%, about 10% to 50%, about 10% to about 40%, about 10% to about 30%, or about 10% to about 20% upon hydration in a gastric environment.

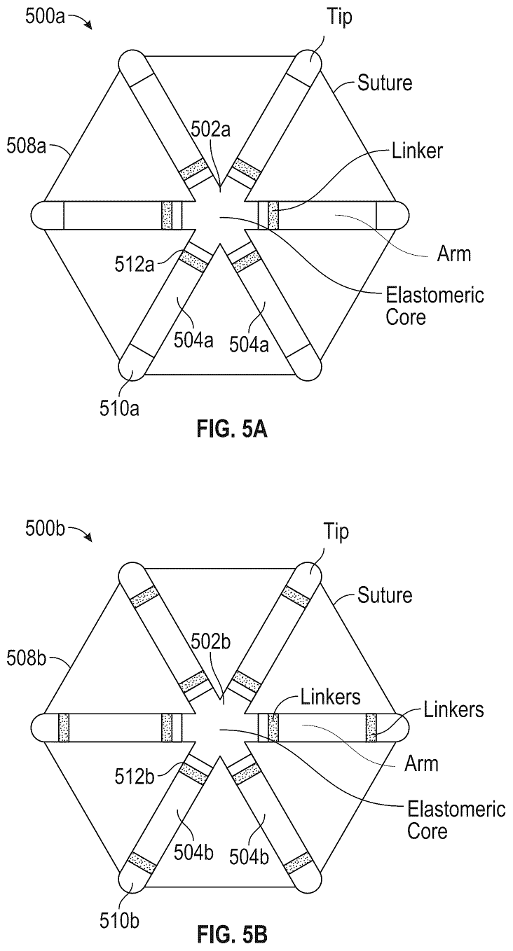

As described, a flexible line may be connected to the distal ends of the arms of the gastric residence system. The flexible line may be connected to the distal end of at least one arm via a separate tip portion. FIGS. 5A and 5B show two different configurations of a gastric residence system having a flexible line connected to the distal end of each arm. FIGS. 5A and 5B show embodiments in which each arm in the gastric residence system includes a separate tip portion. In particular, FIG. 5A shows gastric residence system 500a comprising core 502a and six arms 504a. Each arm 504a comprises a separate tip portion 510a at a distal end. In some embodiments, each arm 504a may be connected to core 502a via one or more linkers 512a. As shown, flexible line 508a connects each arm 504a at separate tip portion 510a. In some embodiments, a single flexible line 508a may wrap circumferentially around gastric residence system 500a, connecting to each arm at separate tip portion 510a. In some embodiments, multiple flexible lines 508a may connect each arm 504a of gastric residence system 500a.

FIG. 5B shows gastric residence system 500b having a core 502b and six arms 504b with a separate tip portion 510b at a distal end of each arm 504b. Unlike gastric residence system 500a of FIG. 5A, gastric residence system 500b comprises one or more linkers 512b connecting each arm 504b to core 502b, as well as one or more linkers 512b connecting two segments of arm 504b. As shown, flexible line 508b connects each arm 504b at separate tip portion 510b. In some embodiments, a single flexible line 508b may wrap circumferentially around gastric residence system 500a, connecting to each arm at separate tip portion 510a. In some embodiments, multiple flexible lines 508b may connect each arm 504b of gastric residence system 500b.

In some embodiments, the flexible line may comprise one or more absorbable flexible line materials. Absorbable flexible line materials generally lose their entire or most of their tensile strength within three months. Examples of absorbable flexible line materials include polyglycolic acid (PGA, e.g., Dexon, Dexon II, Dexon S, Bondek), poly(lactic-co-glycolic acid) (PLGA, e.g., DemeTECH Polyglactin 910), polycaprolactone (PCL), poly(lactic acid) (PLA), PCL-PLA copolymers, polydioxanone, poly(trimethylene carbonate), PCL-poly(glycolic acid) copolymers, Poly(glycerol sebacate), Polyanhydrides, Polyphosphazenes, Poly(alkyl cyanoacrylate)s, poly(amino acids), Poly(propylene fumarate), catgut (collagen derived from sheep intestinal submucosa), reconstituted collagen, poly(glycolide/lactide) random copolymer (e.g., Vicryl), antimicrobial-coated Vicryl (Vicryl Plus), poly-p-dioxanone (e.g., PDS, PDSII, PDS Plus), poly(glycolide/trimethylene carbonate) block copolymer (e.g., Maxon), poly(glycolide/F-caprolactone) (e.g., Monocryl, Monocryl Plus), poly(glycolide/p-dioxanone/trimethylene carbonate) triblock copolymer (e.g., Biosyn), poly(glycolide/ε-caprolactone/trimethlyene carbonate) triblock coppolymer (Caprosyn), polyglytone 6211, 100% poly-L-lactide (Orthodek), polyhydroxyalkanoates (PHA), poly(ethylene glycol)-co-poly(lactic acid) (PELA), poly-4-hydroxybutyrate (e.g., TephaFlex), and/or combinations thereof. A particularly useful flexible line material can comprise PGA. In some embodiments, a flexible line may comprise about 10 to about 100 wt. %, about 10 to about 90 wt. %, about 20 to about 80%, or about 30 to about 70 wt. % absorbable material. In some embodiments, a flexible line may comprise less than about 100 wt. %, less than about 90 wt. %, less than about 80 wt. %, less than about 70 wt. %, less than about 60 wt. %, less than about 50 wt. %, less than about 40 wt. %, less than about 30 wt. %, or less than about 20 wt. % absorbable material. In some embodiments, a flexible line may comprise more than about 10 wt. %, more than about 20 wt. %, more than about 30 wt. %, more than about 40 wt. %, more than about 50 wt. %, more than about 60 wt. %, more than about 70 wt. %, more than about 80 wt. % absorbable material, or more than about 90 wt. % absorbable material. In some embodiments, a flexible line may comprise 100 wt. % absorbable material.

In some embodiments, the absorbable material may be crystalline, semi-crystalline, or amorphous. The degree of crystallinity may impact the speed and/or degree to which the absorbable material loses its tensile strength.

In some embodiments, the absorbable material is PLGA. The ratio of poly(glycolic acid) (PGA) to poly(lactic acid) (PLA) in the PLGA can be varied. In some embodiments, the molar ratio of PGA to PLA may be about 95:5 to about 40:60. In some embodiments, the molar ratio of PGA to PLA may be 95:5, 90:10, 85:15, 80:20, 75:25, 70:30, 65:35, 60:40, 55:45, 50:50, 45:55, or 40:60. In some embodiments, the intrinsic viscosity of the PLGA may be about 0.5-3 dL/g. In some embodiments, the intrinsic viscosity of the PLGA may be at least 0.5 dL/g, at least 1.0 dL/g, at least 1.5 dL/g, at least 2.0 dL/g, or at least 2.5 dL/g. In some embodiments, the intrinsic viscosity of the PLGA may be less than 3.0 dL/g, less than 2.5 dL/g, less than 2.0 dL/g, less than 1.5 dL/g, or less than 1.0 dL/g.

In some embodiments, the flexible line may comprise one or more non-absorbable flexible line materials. Non-absorbable materials retain most of their initial strength for longer than two to three months. Examples of non-absorbable flexible line materials include natural fibers (e.g., silk, cotton, linen) and man-made fibers (e.g., polyethylene terephthalate (PET), polypropylene (PP), ultra-high molecular weight polyethylene (UHMWPE), polyamide (e.g., Nylon 66, Nylon 6), polyester, poly(ether ester), polytetrafluoroethylene (PTFE), polyvinylidine fluoride (PVDF), stainless steel). In some embodiments, a flexible line may comprise about 10 wt. % to about 90 wt. %, about 20 wt. % to about 80 wt. %, about 30 wt. % to about 70 wt. %, or about 40 wt. % to about 60% wt. % non-absorbable material. In some embodiments, a flexible line may comprise less than about 90 wt. %, less than about 80 wt. %, less than about 70 wt. %, less than about 60 wt. %, less than about 50 wt. %, less than about 40 wt. %, less than about 30 wt. %, or less than about 20 wt. % non-absorbable material. In some embodiments, a flexible line my comprise more than about 10 wt. %, more than about 20 wt. %, more than about 30 wt. %, more than about 40 wt. %, more than about 50 wt. %, more than about 60 wt. %, more than about 70 wt. %, or more than about 80 wt. % non-absorbable material.

In some embodiments, the flexible line may further comprise a plasticizer. Suitable plasticizers may include propylene glycol, P407, triethyl citrate, triacetin, dibutyl sebacate, and/or polyethylene glycol. In some embodiments, a flexible line may include about 0.1 to about 20 wt. % plasticizer, about 1 to about 10 wt. % plasticizer, or about 2 to about 8 wt. % plasticizer. In some embodiments, a flexible line may comprise less than about 20 wt. %, less than about 15 wt. %, less than about 10 wt. %, less than about 5 wt. %, or less than about 1 wt. % plasticizer. In some embodiments, a flexible line may comprise more than about 0.1 wt. %, more than about 1 wt. %, more than about 5 wt. %, more than about 10 wt. %, or more than about 15 wt. % plasticizer.

In some embodiments, the flexible line may further comprise one or more drugs. For example, the flexible line may be extruded or co-extruded using a drug-containing rod stock.

In some embodiments, the flexible line may have a cross-section in the shape of a circle, an oval, an annulus, a square, a rectangle, or an irregular shape (e.g., a braided, knotted, or barbed shape). The thickness or diameter of the cross-section may be about 0.05 mm to about 1 mm, about 0.1 mm to about 0.9 mm, about 0.2 mm to about 0.8 mm, or about 0.3 mm to about 0.6 mm. In some embodiments, the thickness or diameter of the cross-section may be less than about 1 mm, less than about 0.8 mm, less than about 0.6 mm, less than about 0.4 mm, less than about 0.2 mm, or less than about 0.1 mm. In some embodiments, the thickness or diameter of the cross-section may be more than about 0.05 mm, more than about 0.1 mm, more than about 0.2 mm, more than about 0.4 mm, more than about 0.6 mm, or more than about 0.8 mm.

In some embodiments, the flexible line may have a cross-sectional area of about 0.005 mm² to about 3.14 mm² or about 0.628 mm² to about 3.14 mm². In some embodiments, the cross-sectional area may be at least about 0.005 mm², at least about 0.01 mm², at least about 0.02 mm², at least about 0.03 mm², at least about 0.04 mm², 0.05 mm², at least about 0.1 mm², at least about 0.2 mm², at least about 0.3 mm², at least about 0.4 mm², at least about 0.5 mm², at least about 0.6 mm², at least about 0.628 mm², at least about 0.7 mm², at least about 0.8 mm², at least about 0.9 mm², at least about 1.0 mm², at least about 1.5 mm², at least about 2.0 mm², at least about 2.5 mm², or at least about 3.0 mm². In some embodiments, the cross-sectional area may be less than about 3.14 mm², less than about 3.0 mm², less than about 2.5 mm², less than about 1.5 mm², or less than about 1.0 mm², less than about 0.9 mm², less than about 0.8 mm², less than about 0.7 mm², less than about 0.628 mm², less than about 0.6 mm², less than about 0.5 mm², less than about 0.4 mm², less than about 0.3 mm², less than about 0.2 mm², less than about 0.1 mm², less than about 0.05 mm², less than about 0.04 mm², less than about 0.03 mm², less than about 0.02 mm², or less than about 0.01 mm². In some embodiments, the cross-sectional area may be at least about 0.005 mm² to about 2.5 mm², at least about 0.01 mm² to about 2.5 mm², at least about 0.05 mm² to about 2.5 mm², at least about 0.1 mm² to about 2.5 mm², at least about 0.5 mm² to about 2.5 mm², at least about 0.628 mm² to about 2.5 mm², at least about 0.8 mm² to about 2.5 mm², at least about 1.0 mm² to about 2.5 mm², at least about 1.2 mm² to about 2.5 mm², at least about 1.2 mm² to about 2.5 mm², at least about 1.4 mm² to about 2.5 mm², at least about 1.6 mm² to about 2.5 mm², at least about 1.8 mm² to about 2.5 mm², at least about 2.0 mm² to about 2.5 mm², at least about 2.2 mm² to about 2.5 mm², or at least about 2.4 mm² to about 2.5 mm². In some embodiments, the cross-sectional area may be at least about 0.005 mm² to about 2.4 mm², at least about 0.005 mm² to about 2.2 mm², at least about 0.005 mm² to about 2.0 mm², at least about 0.005 mm² to about 1.8 mm², at least about 0.005 mm² to about 1.6 mm², at least about 0.005 mm² to about 1.4 mm², at least about 0.005 mm² to about 1.2 mm², at least about 0.005 mm² to about 1.0 mm², or at least about 0.005 mm² to about 0.8 mm². In some embodiments, the cross-sectional area may be at least about 0.628 mm² to about 2.4 mm², at least about 0.628 mm² to about 2.2 mm², at least about 0.628 mm² to about 2.0 mm², at least about 0.628 mm² to about 1.8 mm², at least about 0.628 mm² to about 1.6 mm², at least about 0.628 mm² to about 1.4 mm², at least about 0.628 mm² to about 1.2 mm², at least about 0.628 mm² to about 1.0 mm², or at least about 0.628 mm² to about 0.8 mm².

In some embodiments, the physical configuration of the flexible line may comprise a single-stranded flexible line such as a smooth, barbed, or knotted monofilament. Barbs and/or knots may increase adhesion between the flexible line and the separate tip portions and/or distal ends of the arms of the gastric residence system. For example, barbs may be created in a flexible line by making shallow radial or angled cuts into a side of the flexible line. The barbs may be rigid and prominent enough to improve adhesion but flexible and small enough to avoid irritating the stomach lining of a subject when the gastric residence system is deployed in a gastric environment.

Alternatively, the flexible line may be a multi-stranded flexible line (e.g., a flexible line comprising two or more twisted strands, two or more knotted strands, and/or a two or more braided strands). A preferred flexible line configuration is braided. In some embodiments, braided configurations may increase the roughness and/or surface area of the flexible line compared to single-stranded flexible line configurations, which may increase adhesion between the flexible line and the separate tip portions and/or distal ends of the arms of the gastric residence system. Increasing flexible line adhesion may prevent premature passage of the gastric residence system through the pylorus.

Braided flexible line configurations may be braided using a machine or by hand. Braided flexible line configurations can be characterized by their braid angle, braid length, braid construction, and number of strands. Braid angle is the angle between the central axis of the braid and a strand of the braid. Braid length is the length between repeating units of the braid. Braid construction is the manner in which strands of a braid are combined.

In some embodiments, a braided configuration may have a braid angle of about 10 to about 40 degrees. In some embodiments, a braided configuration may have a braid angle of less than about 40 degrees, less than about 35 degrees, less than about 30 degrees, less than about 25 degrees, less than about 20 degrees, or less than about 15 degrees. In some embodiments, a braided configuration may have a braid angle of more than about 10 degrees, more than about 15 degrees, more than about 20 degrees, more than about 25 degrees, more than about 30 degrees, or more than about 35 degrees.

In some embodiments, a braided configuration may have a braid length of about 0.5 mm to about 3.1 mm. In some embodiments, a braided configuration may have a braid length less than about 3.1 mm, less than about 2.6 mm, less than about 2.1 mm, less than about 1.6 mm, less than about 1.1 mm, or less than about 0.6 mm. In some embodiments, a braided configuration may have a braid length of more than about 0.5 mm, more than about 1.0 mm, more than about 1.5 mm, more than about 2.0 mm, more than about 2.5 mm, or more than about 3.0 mm.

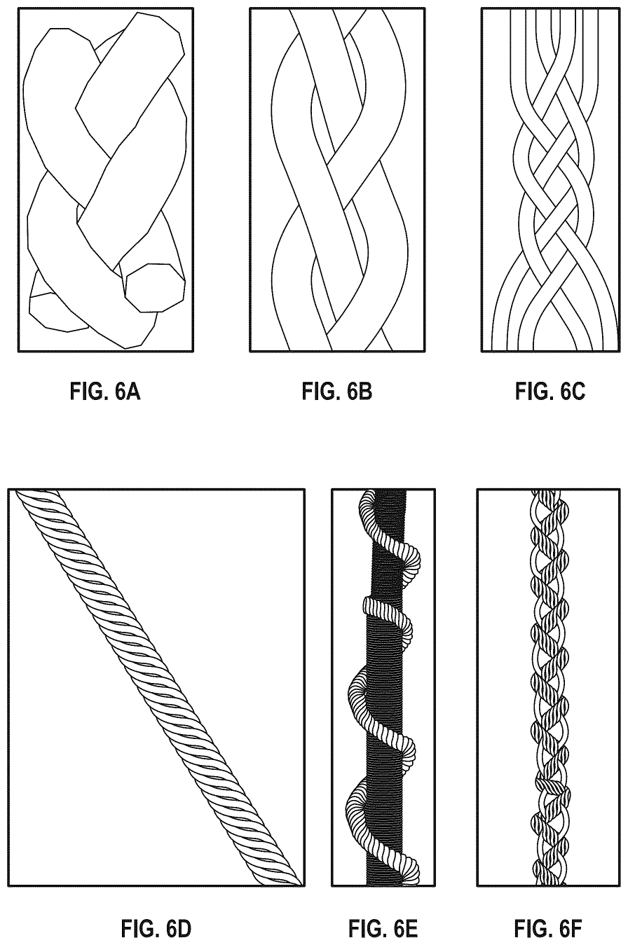

In some embodiments, the braid construction of a braided flexible line may be a standard braid (e.g., a flexible line constructed by interlacing three or more strands in a repeating pattern). Alternatively, the braid construction of a braided flexible line may be a variation of a standard braid (e.g., a flexible line comprising a braided core and a braided sheath surrounding the core). Exemplary braided flexible line configurations are shown in FIGS. 6A-6F. FIG. 6A shows a standard three-stranded braided configuration. FIG. 6B shows a standard three-stranded braided configuration with a looser braid than the configuration shown in FIG. 6A. FIG. 6C shows a standard four-stranded braided configuration. FIG. 6D shows a candy-stripe knot braid. FIG. 6E shows a Chinese step ladder braid. FIG. 6F shows a lattice knot braid.

In some embodiments, a braided configuration may include up to 1000 strands. As used herein, a “strand” refers to a monofilament. A strand may be smooth or may have barbs and/or knots, which may increase the roughness and/or surface area of the strand. A braided configuration may include a plurality of individual strands braided together. Alternatively, a braided configuration may include a plurality of braids that are braided together. For example, a first plurality of strands may be braided together to form a first braid, a second plurality of strands may be braided together to form a second braid, and a third plurality of strands may be braided together to form a third braid. The first braid, the second braid, and the third braid may then be braided together to form a fourth braid. In some embodiments, braiding a plurality of braids together may increase roughness and/or surface area of the resulting braid more than braiding the equivalent number of individual strands (i.e., monofilaments) together. For example, braiding together three braids each having three individual strands may increase roughness and/or surface area more than braiding together nine individual strands without first creating three braids.

In some embodiments, a flexible line may be constructed by performing multiple braiding passes. For example, a first pass may involve creating three braids, each braid having three strands. A second pass may involve braiding together the three braids. This process may be repeated any number of times. In some embodiments, a braided flexible line may be constructed by performing at least one braiding pass, at least two braiding passes, at least three braiding passes, at least four braiding passes, at least five braiding passes, at least six braiding passes, at least seven braiding passes, at least eight braiding passes, at least nine braiding passes, or at least ten braiding passes. In some embodiments, a braided flexible line may be constructed by performing less than eleven braiding passes, less than ten braiding passes, less than nine braiding passes, less than eight braiding passes, less than seven braiding passes, less than six braiding passes, less than five braiding passes, less than four braiding passes, less than three braiding passes, or less than two braiding passes.

In some embodiments, a braided configuration may include between 2-1000 total strands. In some embodiments, a braided configuration may include at least two strands, at least three strands, at least four strands, at least five strands, at least ten strands, at least twenty strands, at least thirty strands, at least forty strands, at least fifty strands, at least 100 strands, at least 200 strands, at least 300 strands, at least 400 strands, at least 500 strands, at least 600 strands, at least 700 strands, at least 800 strands, or at least 900 strands. In some embodiments, a braided configuration may include less than 1000 strands, less than 900 strands, less than 800 strands, less than 700 strands, less than 600 strands, less than 500 strands, less than 400 strands, less than 300 strands, less than 200 strands, less than 100 strands, less than fifty strands, less than forty strands, less than thirty strands, less than twenty strands, less than ten strands, less than five strands, less than four strands, or less than three strands.

In some embodiments, the flexible line may be characterized by its surface roughness. Surface roughness measures the deviation of a surface from a mean height. Surface roughness may be measured by imaging a flexible line using Raman spectroscopy in order to generate a topographical map of the surface of the flexible line. Raman analysis may be performed on the topological map to generate a set of x, y, and z values for each coordinate on the map. Surface roughness may then be calculated by taking the root mean square of the z values of the map. In some embodiments, the flexible line may have a surface roughness of about 5 μm to about 400 μm or about 50 μm to about 250 μm. In some embodiments, the flexible line may have a surface roughness of less than about 400 μm, less than about 350 μm, less than about 300 μm, less than about 250 μm, less than about 200 μm, less than about 150 μm, less than about 100 μm, less than about 50 μm, less than about 25 μm, less than about 20 μm, less than about 15 μm, or less than about 10 μm. In some embodiments, the flexible line may have a surface roughness of more than about 5 μm, more than about 10 μm, more than about 15 μm, more than about 20 μm, more than about 25 μm, more than about 50 μm, more than about 100 μm, more than about 150 μm, more than about 200 μm, more than about 250 μm, more than about 300 μm, or more than about 350 μm. In some embodiments, the flexible line may have a surface roughness of about 5 μm to about 200 μm, about 5 μm to about 150 μm, about 5 μm to about 100 μm, about 100 μm to about 400 μm, about 150 μm to about 400 μm, or about 200 μm to about 400 μm. In some embodiments, the suture may have a surface roughness of about 50 μm to about 200 μm, about 50 μm to about 150 μm, about 50 μm to about 100 μm, about 100 μm to about 250 μm, about 150 μm to about 250 μm, or about 200 μm to about 250 μm.

In some embodiments, the flexible line may comprise a coating. Suitable coatings may include insoluble coatings or water-soluble coatings. Exemplary insoluble coatings may include polycaprolactone (DemeSORB), polyglactin 370 (Vicryl), polycaprolate (Dexon II), and polyhydroxybutyrate. Exemplary water-soluble coatings may include poloxamer 188 (Dexon Plus). In some embodiments, coatings may include poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, or mixtures thereof. A coating may increase adhesion of the flexible line to the arms of the gastric residence system.

In some embodiments, a corona treatment may be applied to the flexible line to alter the surface properties of the flexible line. A high voltage electrical discharge (e.g., about 10,000-30,000 V) may be applied to the flexible line. The electrical discharge may alter the properties of the surface of the flexible line. For example, the electrical discharge may increase wettability of the flexible line and/or the surface area of the flexible line, which may result in improved adhesion of the flexible line to the arms of the gastric residence system.

A length of the flexible line can be measured as a length between each arm of the gastric residence system, or, for embodiments comprising a single flexible line wrapping the circumference of the gastric residence system, the entire length of said circumferentially-wrapped flexible line. Either way, the length of a flexible line depends on the size of the gastric residence system and the number of arms. For example, for a stellate-shape gastric residence system comprising six arms, the length of a circumferentially-wrapped single flexible line may be about 60 mm to about 200 mm, about 75 mm to about 175 mm, about 100 mm to about 150 mm, about 110 mm to about 140 mm, or about 120 mm to about 130 mm in length. In some embodiments, the length of a circumferentially-wrapped single flexible line may be at least 60 mm, at least 70 mm, at least 80 mm, at least 90 mm, at least 100 mm, at least 110 mm, at least 120 mm, at least 130 mm, at least 140 mm, at least 150 mm, at least 160 mm, at least 170 mm, at least 180 mm, or at least 190 mm. In some embodiments, the length of a circumferentially-wrapped single flexible line may be less than 200 mm, less than 190 mm, less than 180 mm, less than 170 mm, less than 160 mm, less than 150 mm, less than 140 mm, less than 130 mm, less than 120 mm, less than 110 mm, less than 100 mm, less than 90 mm, less than 80 mm, or less than 70 mm. The length of the flexible line between any two adjacent arms of the six arms may be about 15 mm to about 30 mm, about 18 mm to about 24 mm, or about 20 mm to about 22 mm. In some embodiments, the length of the flexible line between any two adjacent arms may be at least 15 mm, at least 18 mm, at least 20 mm, at least 22 mm, at least 24 mm, at least 26 mm, or at least 28 mm. In some embodiments, the length of the flexible line between any two adjacent arms may be less than 30 mm, less than 28 mm, less than 26 mm, less than 24 mm, less than 22 mm, less than 20 mm, less than 18 mm, or less than 16 mm.

The force required to separate a flexible line from a separate tip portion and/or distal end of an arm of the gastric residence system may be quantified using the Two Arm Pull (TAPS) test, described in detail in the “Testing Methods” section below. In some embodiments, the force required to separate a flexible line from its corresponding separate tip portion or distal end may be about 4 N to about 25 N. In some embodiments, the force required to separate a flexible line from its corresponding separate tip portion or distal end may be less than about 25 N, less than about 20 N, less than about 15 N, less than about 10 N, or less than about 5 N. In some embodiments, the force required to separate a flexible line from its corresponding separate tip portion or distal end may be greater than about 4 N, greater than about 10 N, greater than about 15 N, or greater than about 20 N. In some embodiments, the force required to separate a flexible line from its corresponding separate tip portion or distal end may decrease the longer the gastric residence system stays in a gastric environment.

In some embodiments, the flexible line may be configured to degrade within a predetermined time period in order to aid passage of the gastric residence system through the pylorus. As used in this context, “degradation” of the flexible line refers to substantial weakening of the flexible line. The flexible line may dissolve or may become weak enough such that it no longer provides resistance to passage through the pylorus (e.g., the flexible line may break or may separate from the arms of the gastric residence system). The flexible line may be configured to degrade within about 7-14 days in a gastric environment. In some embodiments, the flexible line may be configured to degrade in less than 14 days, less than 13 days, less than 12 days, less than 11 days, less than 10 days, less than 9 days, or less than 8 days in a gastric environment. In some embodiments, the flexible line may be configured to degrade in more than 7 days, more than 8 days, more than 9 days, more than 10 days, more than 11 days, more than 12 days, or more than 13 days in a gastric environment.

In some embodiments, the flexible line may be sterilized. Sterilization may have the effect of artificially aging the flexible line by morphologically degrading the flexible line. The flexible line may be sterilized prior to deploying a gastric residence system including the flexible line in a gastric environment (e.g., prior to forming the gastric residence system). Sterilizing the flexible line may provide the flexible line with a faster absorption profile (i.e., sterilization may decrease the amount of time required for the flexible line to degrade in a gastric environment). In some embodiments, sterilizing the flexible line may include heating the flexible line at a high temperature, subjecting the flexible line to a high pressure, irradiating the flexible line with radiation (e.g., gamma radiation), autoclaving the flexible line, treating the flexible line with ethylene oxide, performing a plasma treatment (e.g., radio-frequency glow discharge), or any other suitable sterilization method.

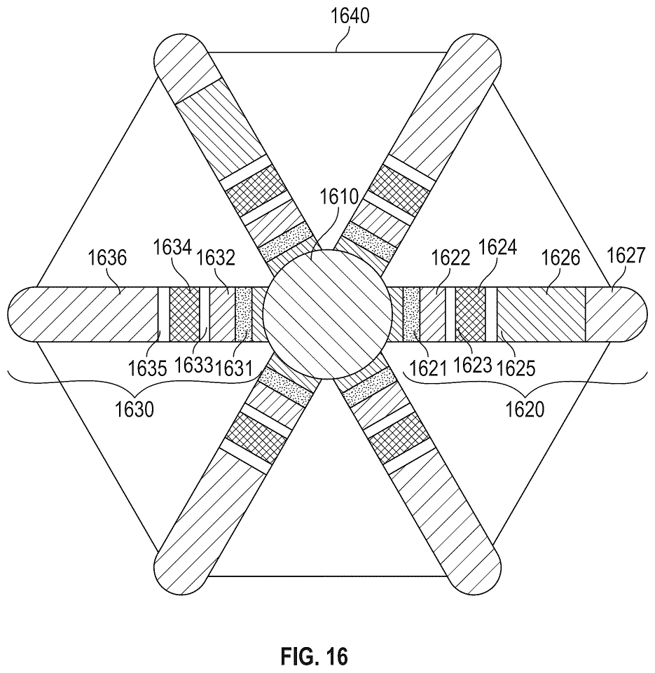

FIG. 16 shows an exemplary gastric residence system with a flexible line, according to some embodiments. The system 1600 comprises a central elastomeric core 1610. The core may comprise liquid silicon rubber overmolded onto a polycarbonate component. The shape of the core 1610 may be an asterisk or a stellate having six short branches to which six arms may attach. In some embodiments, the liquid silicon rubber component of the core 1610 may be about 5-10 mm, about 6-9 mm, about 7-8 mm, or about 7.5 mm in length at its longest (e.g., from a tip of a first branch to a tip of a second branch directly opposite the first branch). In some embodiments, the polycarbonate component of the core 1610 may be about 7-15 mm, about 10-12 mm, or about 11.5 mm in length at its longest. The polycarbonate component may serve as an intercomponent anchor, in that it may extend from the core into one or more additional segments, serving to strengthen the connection between the core and the arms. Examples of intercomponent anchors are described in US Patent Application Publication No. 2019/0262265 at, e.g., FIG. 14A and FIG. 14B, FIG. 36A-FIG. 36E, paragraphs [0031]-[0035], and paragraphs [0294]-[0375]. At least one arm 1620 connected to the core 1610 may include an active pharmaceutical ingredient-containing portion 1626. FIG. 16 shows two such active-pharmaceutical ingredient-containing arms. One or more arms connected to the core 1610 may not contain an active pharmaceutical ingredient-containing portion. FIG. 16 shows four such non-active pharmaceutical ingredient-containing arms. (For the sake of brevity, only one such arm 1630 is labeled in FIG. 16.)

For an arm 1620 including an active pharmaceutical ingredient-containing portion (a drug-eluting arm), a first inert segment 1621 may optionally be connected to a branch of the core 1610. The first inert segment 1621 may comprise polycaprolactone (PCL). In some embodiments, the first inert segment 1621 may be injection molded onto the core 1610 such that inert segment 1621 is a part of the core 1610, rather than a separate segment of the drug-eluting arm 1620.

First inert segment 1621 may be attached to a time-dependent disintegrating matrix segment 1622. In some embodiments, time-dependent disintegrating matrix segment 1622 may comprise one or more of PCL (e.g., PCL12), poly(ethylene oxide) (e.g., PEO100K), a DL-lactide/glycolide copolymer (e.g., 50/50 DL-Lactide/Glycolide copolymer, acid (pDLG 5002A)), and/or ferrosoferric oxide. Time-dependent disintegrating matrix segment 1622 may be configured to degrade in a predictable, time-dependent manner. In some embodiments, degradation of time-dependent disintegrating matrix segment 1622 may not be affected by the varying pH of the gastrointestinal system. Exemplary amounts of the components for the time-dependent disintegrating matrix segment 1622 are provided in the table below.

| Time-dependent | | | |
|---|---|---|---|
| disintegrating | Formulation 1 | Formulation 2 | Formulation 3 |
| matrix segment | (% w/w) | (% w/w) | (% w/w) |
| PCL12 | 35-55 | 40-50 | 42-47 |
| PEO100K | 0.5-3.5 | 1-3 | 1.5-2.5 |
| 50/50 DL-Lactide/ | 40-65 | 45-60 | 50-55 |
| Glycolide copolymer, | | | |
| acid (pDLG 5002A) | | | |
| Ferrosoferric Oxide | 0.001-1 | 0.005-0.5 | 0.01-0.1 |

Time-dependent disintegrating matrix segment 1622 may be attached to a second inert segment 1623. The second inert segment 1623 may comprise radiopaque PCL (rPCL), which may comprise PCL (e.g., Corbion PC17) and bismuth subcarbonate. Exemplary amounts of the components for the second inert segment 1623 are provided in the table below.

| | Formulation 1 | Formulation 2 | Formulation 3 |
|---|---|---|---|
| Second inert segment | (% w/w) | (% w/w) | (% w/w) |
| Corbion PC17 | 60-80 | 65-75 | 68-72 |
| Bismuth subcarbonate | 20-40 | 25-35 | 28-32 |

Second inert segment 1623 may be attached to an enteric disintegrating matrix segment 1624. Enteric disintegrating matrix segment 1624 may comprise one or more of PCL, an enteric polymer (e.g., hydroxypropyl methylcellulose acetate succinate MG grade, i.e., HPMCAS-MG), or poloxamer 407 (P407). Enteric disintegrating matrix segment 1624 may be designed to break down gradually in a controlled manner during the residence period of the system in the stomach and break down more rapidly if the gastric residence system prematurely passes into the small intestine intact. Enteric disintegrating matrix segment 1624 may be relatively resistant to the acidic pH levels of the stomach but may dissolve at the higher pH levels found in the duodenum. Thus, enteric disintegrating matrix segment 1624 may protect against undesired passage of an intact gastric residence system into the small intestine. Exemplary amounts of the components for enteric disintegrating matrix segment 1624 are provided in the table below.

| Enteric | | | |
|---|---|---|---|
| disintegrating | Formulation 1 | Formulation 2 | Formulation 3 |
| matrix segment | (% w/w) | (% w/w) | (% w/w) |
| PCL | 30-55 | 35-50 | 40-45 |
| HPMCAS-MG | 40-70 | 45-65 | 50-60 |
| P407 | 0.1-5 | 0.5-3 | 1-2.5 |

Enteric disintegrating matrix segment 1624 may be attached to a third inert segment 1625. The third inert segment 1625 may have the same composition as the second inert segment 1623, namely rPCL comprising PCL (e.g., Corbion PC17) and bismuth subcarbonate. Exemplary amounts of the components for third inert segment 1625 are provided in the table below.

| | Formulation 1 | Formulation 2 | Formulation 3 |
|---|---|---|---|
| Third inert segment | (% w/w) | (% w/w) | (% w/w) |
| Corbion PC17 | 60-80 | 65-75 | 68-72 |
| Bismuth subcarbonate | 20-40 | 25-35 | 28-32 |

Third inert segment 1625 may be attached to an active pharmaceutical ingredient-containing portion 1626. In some embodiments, active pharmaceutical ingredient-containing portion 1626 may include risperidone. Active pharmaceutical ingredient-containing portion 1626 may further include one or more of PCL (e.g., PCL17), copovidone (e.g., Kollidon VA64), poloxamers (e.g., P407), Vitamin E succinate, colloidal silicon dioxide, or colorants (e.g., FD&C Yellow 5 Alum lake, FD&C Blue 1 Alum lake). Exemplary amounts of the components for active pharmaceutical ingredient-containing portion 1626 are provided in the table below.

| Active pharmaceutical | | | |
|---|---|---|---|
| ingredient-containing | Formulation 1 | Formulation 2 | Formulation |
| portion | (% w/w) | (% w/w) | 3 (% w/w) |
| Risperidone | 20-50 | 25-45 | 30-40 |
| PCL17 | 40-70 | 45-65 | 50-60 |
| VA64 | 1-10 | 2-8 | 4-6 |
| P407 | 0.5-8 | 1-5 | 2-4 |
| Vitamin E succinate | 0.01-2 | 0.05-1.5 | 0.1-1 |
| Silicon dioxide (SiO₂) | 0.01-2 | 0.05-1.5 | 0.1-1 |
| FD&C Yellow 5 Alum | 0.001-1 | 0.005-0.5 | 0.01-0.1 |
| lake (16-18%) | | | |
| FD&C Blue 1 Alum lake | 0.001-1 | 0.005-0.5 | 0.01-0.1 |
| (11-13%) | | | |

In some embodiments, active pharmaceutical ingredient-containing portion 1626 may include about 10% to about 80% by weight active pharmaceutical ingredient (API). In some embodiments, active pharmaceutical ingredient-containing portion 1626 may include at 10% by weight API, at least 20% by weight API, at least 30% by weight API, at least 40% by weight API, at least 50% by weight API, at least 60% by weight API, or at least 70% by weight API. In some embodiments, active pharmaceutical ingredient-containing portion 1626 may include less than 80% by weight API, less than 70% by weight API, less than 60% by weight API, less than 50% by weight API, less than 40% by weight API, less than 30% by weight API, or less than 20% by weight API.

Active pharmaceutical ingredient containing portion 1626 may be connected to a fourth inert segment 1627 (also referred to herein as a “separate tip portion”). In some embodiments, a portion of a flexible line 1640 may be embedded in the fourth inert segment 1627, which may connect the active pharmaceutical ingredient-containing arm 1620 to adjacent arms (e.g., arm 1630). The fourth inert segment 1627 may comprise PCL (e.g., Corbion PC17). In some embodiments, the fourth inert segment 1627 may further comprise one or more of copovidone (e.g., VA64), poloxamers (e.g., P407), or colorants (e.g., FD&C Blue 1 Alum lake (11-13%)). Exemplary amounts of the components for fourth inert segment 1627 are provided in the table below.

| Fourth inert | Formulation 1 | Formulation 2 | Formulation 3 |
|---|---|---|---|
| segment | (% w/w) | (% w/w) | (% w/w) |
| Corbion PC17 | 50-100 | 60-80 | 65-75 |
| VA64 | 20-45 | 25-40 | 30-35 |
| P407 | 0.01-5 | 0.1-3 | 1-2 |
| FD&C Blue 1 Alum | 0.001-2 | 0.005-1 | 0.01-0.5 |
| lake (11-13%) | | | |

Approximate radial lengths of the segments on an exemplary drug eluting arm 1620 are provided in the table below.

| | Radial | | Radial | | Radial | |
|---|---|---|---|---|---|---|
| Segment | length set 1 | | length set 2 | | length set 3 | |
| First inert segment | 0.1-5 | mm | 0.5-3 | mm | 1.3 | mm |
| Time-dependent | 0.1-5 | mm | 0.5-3 | mm | 1.0 | mm |
| disintegrating matrix | | | | | | |
| segment | | | | | | |
| Second inert segment | 0.05-3 | mm | 0.1-1.5 | mm | 0.5 | mm |
| Enteric disintegrating | 0.1-5 | mm | 0.5-3 | mm | 1.85 | mm |
| matrix segment | | | | | | |
| Third inert segment | 0.05-3 | mm | 0.1-1.5 | mm | 0.5 | mm |
| Active pharmaceutical | 3-12 | mm | 5-9 | mm | 7.3 | mm |
| ingredient-containing | | | | | | |
| portion | | | | | | |
| Fourth inert segment | 2-10 | mm | 4-7 | mm | 5.8 | mm |

In some embodiments, each segment of drug eluting arm 1620 may have a triangular cross-section. In some embodiments, the triangular cross-section is an equilateral triangular cross section. The base length of the equilateral triangle of each segment of drug eluting arm 1620 is provided in the table below.

| | Cross-section | Cross-section | Cross-section |
|---|---|---|---|
| | base length | base length | base length |
| Segment | set 1 | set 2 | set 3 |
| First inert segment | 2.5-3.5 mm | 2.9-3.3 mm | 3.3 mm |
| Time-dependent | 2.5-3.5 mm | 2.9-3.3 mm | 3.3 mm |
| disintegrating matrix | | | |
| segment | | | |
| Second inert segment | 2.5-3.5 mm | 2.9-3.3 mm | 3.3 mm |
| Enteric disintegrating | 2.5-3.5 mm | 2.9-3.3 mm | 3.3 mm |
| matrix segment | | | |
| Third inert segment | 2.5-3.5 mm | 2.9-3.3 mm | 3.3 mm |
| Active pharmaceutical | 2.5-3.5 mm | 2.9-3.3 mm | 3.3 mm |
| ingredient-containing | | | |
| portion | | | |
| Fourth inert segment | 2.5-3.5 mm | 2.9-3.3 mm | 3.1 mm |

In some embodiments, drug eluting arm 1620 may comprise a coating. The coating may comprise one or more of PCL (e.g., PCL17), copovidone (e.g., VA64), or magnesium stearate. In some embodiments, the coating may further comprise trace amounts of ethyl acetate used to dissolve PCL17, VA64, and/or magnesium stearate in preparation for coating. The coating may be applied using a pan coater. In some embodiments, the coat may comprise about 2-4% of the weight of the coated drug-eluting arm 1620. Exemplary amounts of the components of the coating are provided in the table below.

| | Formulation 1 | Formulation 2 | Formulation 3 |
|---|---|---|---|
| Coating | (% w/w) | (% w/w) | (% w/w) |
| PCL17 | 60-90 | 65-85 | 70-80 |
| VA64 | 10-40 | 20-30 | 22-27 |
| Magnesium | 0.1-5 | 0.5-4 | 1-3 |
| stearate | | | |

In some embodiments, one arm of the gastric residence system 1600 comprises a drug eluting arm 1620. In some embodiments, two arms of the gastric residence system 1600 comprise a drug eluting arm 1620. In some embodiments, three arms of the gastric residence system 1600 comprise a drug eluting arm 1620. In some embodiments, four arms of the gastric residence system 1600 comprise a drug eluting arm 1620. In some embodiments, five arms of the gastric residence system 1600 comprise a drug eluting arm 1620. In some embodiments, all six arms of the gastric residence system 1600 comprise a drug eluting arm 1620.

In some embodiments, the gastric residence system 1600 comprises one or more arms 1630 that do not include an active pharmaceutical ingredient-containing portion (non-drug eluting arms). For a non-drug eluting arm 1630, a first inert segment 1631 may be connected to a branch of the core 1610. The first inert segment 1631 may comprise PCL. First inert segment 1631 may have the same composition as first inert segment 1621. Like first inert segment 1621, first inert segment 1631 may be injection molded onto the core 1610, such that first inert segment 1631 is a part of the core rather than a separate segment of a non-drug eluting arm 1630.

First inert segment 1631 may be attached to a time-dependent disintegrating matrix segment 1632. Time-dependent disintegrating matrix segment 1632 may have the same composition as time-dependent disintegrating matrix segment 1622, namely one or more of PCL (e.g., PCL12), poly(ethylene oxide) (e.g., PEO100K), a DL-lactide/glycolide copolymer (e.g., 50/50 DL-Lactide/Glycolide copolymer, acid (pDLG 5002A)), and/or ferrosoferric oxide.

Time-dependent disintegrating matrix segment 1632 may be attached to a second inert segment 1633. Second inert segment 1633 may have the same composition as second inert segment 1623, namely PCL (e.g., Corbion PC17) and bismuth subcarbonate.

Second inert segment 1633 may be attached to an enteric disintegrating matrix segment 1634. Enteric disintegrating matrix segment 1634 may have the same composition as enteric disintegrating matrix segment 1624, namely one or more of PCL, an enteric polymer (e.g., HPMCAS-MG), or a poloxamer (e.g., P407).

Enteric disintegrating matrix segment 1634 may be attached to a third inert segment 1635. Third inert segment 1635 may have the same composition as third inert segment 1625, namely PCL (e.g., Corbion PC17) and bismuth subcarbonate.

Third inert segment 1635 may be attached to a fourth inert segment 1636. Fourth inert segment 1636 (also referred to herein as the “distal end” of a non-drug eluting arm) may have the same composition as fourth inert segment 1627 (the “separate tip portion” of a drug-eluting arm), namely one or more of PCL (e.g., Corbion PC17), copovidone (e.g., VA64), poloxamer (e.g., P407), or colorants (e.g., FD&C Blue 1 Alum lake (11-13%)). In some embodiments, a portion of a flexible line 1640 may be embedded in the fourth inert segment.

Approximate radial lengths of the segments on an exemplary non-drug eluting arm 1630 are provided in the table below.

| | Radial | | Radial | | Radial | |
|---|---|---|---|---|---|---|
| Segment | length set 1 | | length set 2 | | length set 3 | |
| First inert segment | 0.1-5 | mm | 0.5-3 | mm | 1.3 | mm |
| Time-dependent | 0.1-5 | mm | 0.5-3 | mm | 1.0 | mm |
| disintegrating matrix | | | | | | |
| segment | | | | | | |
| Second inert segment | 0.05-3 | mm | 0.1-1.5 | mm | 0.5 | mm |
| Enteric disintegrating | 0.1-5 | mm | 0.5-3 | mm | 1.85 | mm |
| matrix segment | | | | | | |
| Third inert segment | 0.05-3 | mm | 0.1-1.5 | mm | 0.5 | mm |
| Fourth inert segment | 5-20 | mm | 10-15 | mm | 13.1 | mm |

In some embodiments, each segment of non-drug eluting arm 1630 may have a triangular cross-section. In some embodiments, the triangular cross-section is an equilateral triangular cross section. The base length of the equilateral triangle of each segment of non-drug eluting arm 1630 is provided in the table below.

| | Cross-section | Cross-section | Cross-section |
|---|---|---|---|
| | base length | base length | base length |
| Segment | set 1 | set 2 | set 3 |
| First inert segment | 2.5-3.5 mm | 2.9-3.3 mm | 3.3 mm |
| Time-dependent | 2.5-3.5 mm | 2.9-3.3 mm | 3.3 mm |
| disintegrating matrix | | | |
| segment | | | |
| Second inert segment | 2.5-3.5 mm | 2.9-3.3 mm | 3.3 mm |
| Enteric disintegrating | 2.5-3.5 mm | 2.9-3.3 mm | 3.3 mm |
| matrix segment | | | |
| Third inert segment | 2.5-3.5 mm | 2.9-3.3 mm | 3.3 mm |
| Fourth inert segment | 2.5-3.5 mm | 2.9-3.3 mm | 3.1 mm |

In some embodiments, the diameter of the gastric residence system, at its widest (e.g., from the distal tip of a first arm to the distal tip of a second arm opposite the first arm), is about 20-60 mm, about 25-55 mm, about 40-50 mm, about 42-48 mm, or about 44-46 mm.

In some embodiments, one arm of the gastric residence system 1600 comprises a non-drug eluting arm 1630. In some embodiments, two arms of the gastric residence system 1600 comprise a non-drug eluting arm 1630. In some embodiments, three arms of the gastric residence system 1600 comprise a non-drug eluting arm 1630. In some embodiments, four arms of the gastric residence system 1600 comprise a non-drug eluting arm 1630. In some embodiments, five arms of the gastric residence system 1600 comprise a non-drug eluting arm 1630. In some embodiments, six arms of the gastric residence system 1600 comprise a non-drug eluting arm 1630.

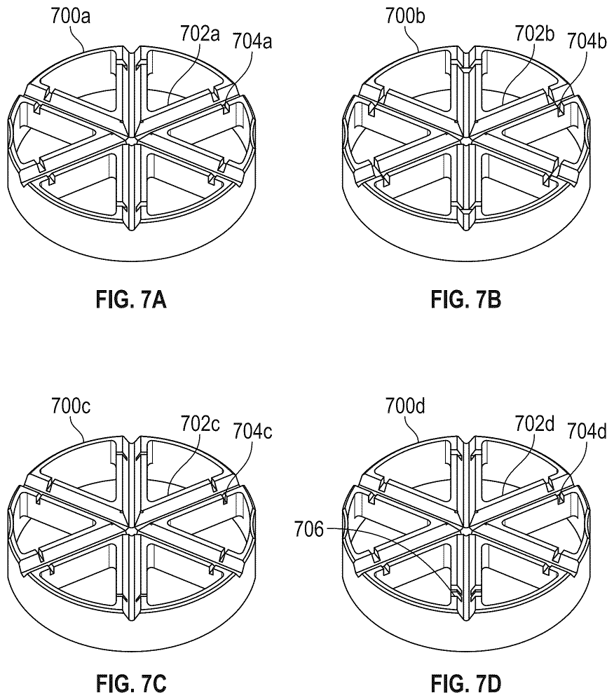

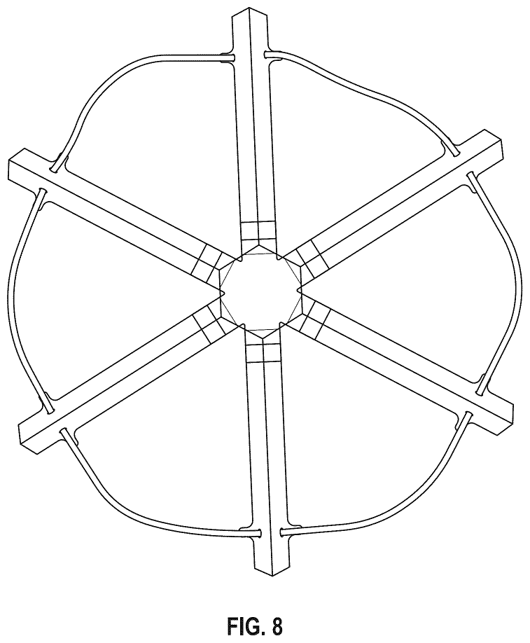

As shown in FIG. 16, a flexible line 1640 may circumferentially connect adjacent arms of the gastric residence system. In some embodiments, the flexible line may comprise polyglycolic acid (PGA). The flexible line may have a diameter of about 0.05-1 mm, about 0.1-0.5 mm, or about 0.2 mm. In some embodiments, the flexible line may comprise a plurality of smaller filaments braided together. The flexible line 1640 may be embedded into each arm of the gastric residence system, for example via the laser welding process described herein with reference to FIGS. 7A-7D.

Gastric residence system 1600 may be folded and enclosed in a sleeve and/or capsule for delivery. In some embodiments, gastric residence system 1600 may first be placed within an inner sleeve before being placed in an outer capsule. The sleeve may comprise hydroxypropyl methylcellulose (HPMC). The sleeve may be a size 0 capsule, cap only. In some embodiments, a capsule may be used in addition to a sleeve. The capsule may comprise HPMC. The capsule may be a Size 00EL capsule. In some embodiments, the capsule may comprise a coating. The capsule coating may comprise one or more of Eudragit E, dibutyl sebacate, or magnesium stearate. The capsule coating may comprise about 35-45 mg per capsule. Exemplary amounts of the components of the capsule coating are provided in the table below.

| | Formulation 1 | Formulation 2 | Formulation 3 |
|---|---|---|---|
| Capsule coating | (% w/w) | (% w/w) | (% w/w) |
| Eudragit E | 70-99 | 80-95 | 85-92 |
| Dibutyl sebacate | 1-10 | 2-8 | 3-5 |
| Magnesium stearate | 1-10 | 2-8 | 3-5 |

### System Dimensions

Once the gastric residence system is assembled, the system must be able to adopt a compacted state with dimensions that enable the patient to swallow the system (or for the system to be introduced into the stomach by alternate means, such as a feeding tube or gastrostomy tube). Typically, the system is held in the compacted state by a container such as a capsule. Upon entry into the stomach, the system is then released from the container and adopts an uncompacted state, that is, an expanded conformation, with dimensions that prevent passage of the system through the pyloric sphincter, thus permitting retention of the system in the stomach.

Accordingly, the system should be capable of being placed inside a standard-sized capsule of the type commonly used in pharmacy. Standard capsule sizes in use in the United States are provided below in the Capsule Table below (see “Draft Guidance for Industry on Size, Shape, and Other Physical Attributes of Generic Tablets and Capsules” at URL www.regulations.gov/#!documentDetail;D=FDA-2013-N-1434-0002). As these are the outer dimensions of the capsule, and as dimensions will vary slightly between capsule manufacturers, the system should be capable of adopting a configuration which is about 0.5 to about 1 mm smaller than the outer diameter shown, and about 1 mm to about 2 mm shorter than the length shown in the Capsule Table.

Capsule Table

| | Outer | |
|---|---|---|
| Capsule | Diameter | Length |
| Size | (mm) | (mm) |
| 000 | 9.9 | 26.1 |
| 00 | 8.5 | 23.3 |
| 0 | 7.6 | 21.7 |
| 1 | 6.9 | 19.4 |
| 2 | 6.3 | 18.0 |
| 3 | 5.8 | 15.9 |
| 4 | 5.3 | 14.3 |
| 5 | 4.9 | 11.1 |

Capsules can be made of materials well-known in the art, such as gelatin or hydroxypropyl methylcellulose. In one embodiment, the capsule is made of a material that dissolves in the gastric environment, but not in the oral or esophageal environment, which prevents premature release of the system prior to reaching the stomach.

In one embodiment, the system will be folded or compressed into a compacted state in order to fit into the capsule, for example, in a manner such as that shown in FIG. 1B. Once the capsule dissolves in the stomach, the system will adopt a configuration suitable for gastric retention, for example, in a manner such as that shown in FIG. 1A. Preferred capsule sizes are 00 and 00e1 (a 00e1-size capsule has the approximate length of a 000 capsule and the approximate width of a 00 capsule), which then places constraints on the length and diameter of the folded system.

Once released from the container, the system adopts an uncompacted state with dimensions suitable to prevent passage of the gastric residence system through the pyloric sphincter. In one embodiment, the system has at least two perpendicular dimensions, each of at least about 2 cm in length; that is, the gastric residence system measures at least about 2 cm in length over at least two perpendicular directions. In another embodiment, the perimeter of the system in its uncompacted state, when projected onto a plane, has two perpendicular dimensions, each of at least about 2 cm in length. The two perpendicular dimensions can independently have lengths of from about 2 cm to about 7 cm, about 2 cm to about 6 cm, about 2 cm to about 5 cm, about 2 cm to about 4 cm, about 2 cm to about 3 cm, about 3 cm to about 7 cm, about 3 cm to about 6 cm, about 3 cm to about 5 cm, about 3 cm to about 4 cm, about 4 cm to about 7 cm, about 4 cm to about 6 cm, about 4 cm to about 5 cm, or about 4 cm to about 4 cm. These dimensions prevent passage of the gastric residence system through the pyloric sphincter. For star-shaped polymers with N arms (where N is greater than or equal to three, such as N=6), the arms can have dimensions such that the system has at least two perpendicular dimensions, each of length as noted above. These two perpendicular dimensions are chosen as noted above in order to promote retention of the gastric residence system.

The system is designed to eventually break apart in the stomach at the end of the desired residence time (residence period), at which point the remaining components of the system are of dimensions that permit passage of the system through the pyloric sphincter, small intestine, and large intestine. Finally, the system is eliminated from the body by defecation, or by eventual complete dissolution of the system in the small and large intestines. Thus, coupling polymers or disintegrating matrices are placed in the gastric residence systems of the invention in a configuration such that, at the end of the desired residence period when the coupling polymers or disintegrating matrices break or dissolve, the uncoupled components of the gastric residence system have dimensions suitable for passage through the pyloric sphincter and elimination from the digestive tract.

### Residence Time

The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks). In one embodiment, the gastric residence system has a residence time of about 21 days (about three weeks), or up to about 21 days (about three weeks). In one embodiment, the gastric residence system has a residence time of about 28 days (about one month), or up to about 28 days (about one month).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

In one embodiment, the gastric residence system has a residence time between about 24 hours and about 21 days. In one embodiment, the gastric residence system has a residence time between about 48 hours and about 21 days. In one embodiment, the gastric residence system has a residence time between about 72 hours and about 21 days. In one embodiment, the gastric residence system has a residence time between about 96 hours and about 21 days. In one embodiment, the gastric residence system has a residence time between about 5 days and about 21 days. In one embodiment, the gastric residence system has a residence time between about 6 days and about 21 days. In one embodiment, the gastric residence system has a residence time between about 7 days and about 21 days. In one embodiment, the gastric residence system has a residence time between about 10 days and about 21 days. In one embodiment, the gastric residence system has a residence time between about 14 days and about 21 days.

In one embodiment, the gastric residence system has a residence time between about 24 hours and about one month (e.g., about 28 days). In one embodiment, the gastric residence system has a residence time between about 48 hours and about one month. In one embodiment, the gastric residence system has a residence time between about 72 hours and about one month. In one embodiment, the gastric residence system has a residence time between about 96 hours and about one month. In one embodiment, the gastric residence system has a residence time between about 5 days and about one month. In one embodiment, the gastric residence system has a residence time between about 6 days and about one month. In one embodiment, the gastric residence system has a residence time between about 7 days and about one month. In one embodiment, the gastric residence system has a residence time between about 10 days and about one month. In one embodiment, the gastric residence system has a residence time between about 14 days and about one month. In one embodiment, the gastric residence system has a residence time between about 21 days and about one month.

The gastric residence system releases a therapeutically effective amount of agent (or salt thereof) during at least a portion of the residence time or residence period during which the system resides in the stomach. In one embodiment, the system releases a therapeutically effective amount of agent (or salt thereof) during at least about 25% of the residence time. In one embodiment, the system releases a therapeutically effective amount of agent (or salt thereof) during at least about 50% of the residence time. In one embodiment, the system releases a therapeutically effective amount of agent (or salt thereof) during at least about 60% of the residence time. In one embodiment, the system releases a therapeutically effective amount of agent (or salt thereof) during at least about 70% of the residence time. In one embodiment, the system releases a therapeutically effective amount of agent (or salt thereof) during at least about 75% of the residence time. In one embodiment, the system releases a therapeutically effective amount of agent (or salt thereof) during at least about 80% of the residence time. In one embodiment, the system releases a therapeutically effective amount of agent (or salt thereof) during at least about 85% of the residence time. In one embodiment, the system releases a therapeutically effective amount of agent (or salt thereof) during at least about 90% of the residence time. In one embodiment, the system releases a therapeutically effective amount of agent (or salt thereof) during at least about 95% of the residence time. In one embodiment, the system releases a therapeutically effective amount of agent (or salt thereof) during at least about 98% of the residence time. In one embodiment, the system releases a therapeutically effective amount of agent (or salt thereof) during at least about 99% of the residence time.

### Evaluation of Release Characteristics

The release characteristics of agent from segments, arms, and gastric residence systems can be evaluated by various assays. Assays for agent release are described in detail in the examples. Release of agent in vitro from segments, arms, and gastric residence systems can be measured by immersing a segment, arm, or gastric residence system in a liquid, such as water, 0.1N HCl, fasted state simulated gastric fluid (FaSSGF), or fed state simulated gastric fluid (FeSSGF). In one embodiment, fasted state simulated gastric fluid (FaSSGF) is used for release assays. Simulated gastric fluid indicates either fasted state simulated gastric fluid (FaSSGF) or fed state simulated gastric fluid (FeSSGF); when a limitation is specified as being measured in simulated gastric fluid (SGF), the limitation is met if the limitation holds in either fasted state simulated gastric fluid (FaSSGF) or fed state simulated gastric fluid (FeSSGF). For example, if a segment is indicated as releasing at least 10% of an agent over the first 24 hours in simulated gastric fluid, the limitation is met if the segment releases at least 10% of the agent over the first 24 hours in fasted state simulated gastric fluid, or if the segment releases at least 10% of the agent over the first 24 hours in fed state simulated gastric fluid.

Ethanol burst release is typically measured by immersing a segment, arm, or gastric residence system in a solution of 40% ethanol and 60% fasted state simulated gastric fluid for one hour, followed by immersing the same segment, arm, or gastric residence system in 100% fasted state simulated gastric fluid for the remainder of the test period, and measuring release of agent at appropriate time points. This test is designed to simulate the effects of consumption of alcoholic beverages by a patient having a gastric residence system of the invention deployed in the patient's stomach.

While in vitro tests can be performed using segments, arms, or gastric residence systems, use of segments for in vitro tests is most convenient for rapid evaluation of the release characteristics. When in vitro tests are done to compare release rates under different conditions (such as release in 100% FaSSGF versus release in 40% ethanol/60% FaSSGF), the comparison solutions are kept at the same temperature, such as room temperature, 25° C., or 37° C.

In vivo tests can be performed in animals such as dogs (for example, beagle dogs or hound dogs), non-human primates (e.g., monkeys), and swine. For in vivo tests, a gastric residence system is used, since an individual segment or arm would not be retained in the stomach of the animal. Blood samples can be obtained at appropriate time points, and, if desired, gastric contents can be sampled by cannula or other technique.

Clinical trials in humans, conducted in accordance with appropriate laws, regulations, and institutional guidelines, also provide in vivo data.

### Gastric Delivery Pharmacokinetics for Gastric Residence Systems

The gastric residence systems of the invention provide for high bioavailability of the agent as measured by AUC_{inf} after administration of the systems, relative to the bioavailability of a conventional oral formulation of the agent. The systems also provide for maintenance of an approximately constant plasma level or a substantially constant plasma level of the agent.

Relative bioavailability, FREL, of two different formulations, formulation A and formulation B, is defined as:

$${F}_{REL} = 100 \times ( {AUC}_{A} \times {Dose}_{B} ) / ( {AUC}_{B} \times {Dose}_{A} )$$

where AUC_{A} is the area under the curve for formulation A, AUC_{B} is the area under the curve for formulation B, Dose_{A} is the dosage of formulation A used, and Dose_{B} is the dosage of formulation B used. AUC, the area under the curve for the plot of agent plasma concentration versus time, is usually measured at the same time (t) after administration of each formulation, in order to provide the relative bioavailability of the formulations at the same time point. AUC_{inf} refers to the AUC measured or calculated over “infinite” time, that is, over a period of time starting with initial administration, and ending where the plasma level of the agent has dropped to a negligible amount.

In one embodiment, the substantially constant plasma level of agent provided by the gastric residence systems of the invention can range from at or above the trough level of the plasma level of agent when administered daily in a conventional oral formulation (that is, Cₘᵢₙ of agent administered daily in immediate-release formulation) to at or below the peak plasma level of agent when administered daily in a conventional oral formulation (that is, Cₘₐₓ of agent administered daily in immediate-release formulation). In some embodiments, the substantially constant plasma level of agent provided by the gastric residence systems of the invention can be about 50% to about 90% of the peak plasma level of agent when administered daily in a conventional oral formulation (that is, Cₘₐₓ of agent administered daily in immediate-release formulation). The substantially constant plasma level of agent provided by the gastric residence systems of the invention can be about 75% to about 125% of the average plasma level of agent when administered daily in a conventional oral formulation (that is, Cₐᵥₑ of agent administered daily in immediate-release formulation). The substantially constant plasma level of agent provided by the gastric residence systems of the invention can be at or above the trough level of plasma level of agent when administered daily in a conventional oral formulation (that is, Cₘᵢₙ of agent administered daily in immediate-release formulation), such as about 100% to about 150% of Cₘᵢₙ.

The gastric residence systems of the invention can provide bioavailability of agent released from the system of at least about 50%, at least about 60%, at least about 70%, or at least about 80% of that provided by an immediate release form comprising the same amount of agent. As indicated above, the bioavailability is measured by the area under the plasma concentration-time curve (AUC_{inf}).

### Dissolution Profile, Bioavailability and Pharmacokinetics for Gastric Residence Systems

The gastric residence systems described herein provide a steady release of an agent or a pharmaceutically acceptable salt thereof over an extended period of time. The systems are designed to release a therapeutically effective amount of an agent or salt thereof over the period of residence in the stomach. The release of agent (or salt thereof) can be measured in vitro or in vivo to establish the dissolution profile (elution profile, release rate) of the agent (or salt thereof) from a given residence system in a specific environment. The dissolution profile can be specified as a percentage of the original amount of agent (or salt thereof) present in the system which elutes from the system over a given time period.

Thus, in some embodiments, the agent (or salt thereof) contained in a gastric residence system can have a dissolution profile of 10-20% release between zero hours and 24 hours in a given environment. That is, over the 24-hour period after initial introduction of the gastric residence system into the environment of interest, 10-20% of the initial agent (or salt thereof) contained in the system elutes from the system.

The environment of interest can be 1) the stomach of a patient (that is, an in vivo environment), or 2) simulated gastric fluid (that is, an in vitro environment).

The gastric residence systems of the invention provide for high bioavailability of the agent (or salt thereof) as measured by AUC_{inf} after administration of the systems, relative to the bioavailability of a conventional oral formulation of the agent (or salt thereof). The systems also provide for maintenance of a substantially constant plasma level of the agent (or salt thereof).

Parameters of interest for release include the linearity of release over the residence period of the gastric residence systems, the standard deviation of release over the residence period (which is related to linearity of release; a standard deviation of zero indicates that release is linear over the entire residence period), the release over the initial six hours of residence (that is, burst release upon initial administration), and total release of agent (or salt thereof) over the residence period. In one embodiment, the residence period is seven days, although other periods, such as two, three, four, five, six, eight, nine, ten, 11, 12, 13, or 14 days can be used.

Linearity of agent (or salt thereof) release over the residence period refers to the amount released during each 24-hour period of residence. For a seven-day period of residence, it is desirable that about the amount of agent (or salt thereof) is released each day, i.e., that linearity of agent (or salt thereof) release is maximized. This will minimize the standard deviation of daily agent or agent salt release over the residence period. In some embodiments, the gastric release systems have a variation (or a standard deviation) for daily agent (or salt thereof) release of less than about 100%, less than about 90%, less than about 80%, less than about 70%, less than about 60%, less than about 50%, less than about 40%, less than about 30%, less than about 25%, less than about 20%, less than about 15%, less than about 10%, or less than about 5%, over the period of residence. In some embodiments, the period of residence can be about three days, about seven days, about ten days, or about two weeks.

Minimization of burst release, that is, release over the initial period of residence (such as six hours, twelve hours, or 24 hours after administration of a gastric residence system) is desirable in order to maintain a predictable and steady release profile. If T is the total agent (or salt thereof) release over the residence period (in units of mass), and D is the number of days of the residence period, then completely linear release would mean that about T/D mass of agent (or salt thereof) is released per day. If the period over which burst release is measured is the first six hours, then a linear release profile will result in 0.25×T/D mass of agent (or salt thereof) released during the first six hours. In percentage terms of the total amount of agent (or salt thereof) released over the residence period of D days, linear release would be about 100/D % of agent (or salt thereof) per day, and a linear release over the first six hours would be 25/D %. (Note that 100% in this context indicates the total amount of agent (or salt thereof) released, regardless of how much agent (or salt thereof) is contained in the initial formulation.) Thus, for a seven day residence period, linear release over the first six hours would be about 3.6% of the total amount of agent (or salt thereof) released over the seven-day period.

In some embodiments, during the initial six hours of residence after administration the gastric residence systems release about 0.2 to about 2 times T/D of the total mass of agent (or salt thereof) T released over the residence period of D days, or about 0.2 to about 1.75 times T/D of the total mass of agent (or salt thereof) T released over the residence period of D days, or about 0.2 to about 1.5 times T/D of the total mass of agent (or salt thereof) T released over the residence period of D days, or about 0.2 to about 1.25 times T/D of the total mass of agent (or salt thereof) T released over the residence period of D days, or about 0.2 to about 1 times T/D of the total mass of agent (or salt thereof) T released over the residence period of D days, or about 0.2 to about 0.8 times T/D of the total mass of agent (or salt thereof) T released over the residence period of D days, or about 0.2 to about 0.75 times T/D, or about 0.2 to about 0.7 times T/D, or about 0.2 to about 0.6 times T/D, or about 0.2 to about 0.5 times T/D, or about 0.2 to about 0.4 times T/D, or about 0.2 to about 0.3 times T/D, or about 0.25 to about 2 times T/D, or about 0.3 to about 2 times T/D, or about 0.4 to about 2 times T/D, or about 0.5 to about 2 times T/D, or about 0.6 to about 2 times T/D, or about 0.7 to about 2 times T/D, or about 0.25 to about 1.5 times T/D, or about 0.3 to about 1.5 times T/D, or about 0.4 to about 1.5 times T/D, or about 0.5 to about 1.5 times T/D, or about 0.6 to about 1.5 times T/D, or about 0.7 to about 1.5 times T/D, or about 0.25 to about 1.25 times T/D, or about 0.3 to about 1.25 times T/D, or about 0.4 to about 1.25 times T/D, or about 0.5 to about 1.25 times T/D, or about 0.6 to about 1.25 times T/D, or about 0.7 to about 1.25 times T/D, or about 0.25 to about 1 times T/D, or about 0.3 to about 1 times T/D, or about 0.4 to about 1 times T/D, or about 0.5 to about 1 times T/D, or about 0.6 to about 1 times T/D, or about 0.7 to about 1 times T/D, or about 0.25 times T/D, or about 0.25 to about 0.8 times T/D, or about 0.3 to about 0.8 times T/D, or about 0.4 to about 0.8 times T/D, or about 0.5 to about 0.8 times T/D, or about 0.6 to about 0.8 times T/D, or about 0.7 to about 0.8 times T/D, or about 0.8 times T/D, about 1 times T/D, about 1.25 times T/D, about 1.5 times T/D, or about 2 times T/D.

In some embodiment of the gastric residence systems, during the initial six hours of residence after administration the gastric residence systems release about 2% to about 10% of the total mass of agent (or salt thereof) released over the residence period, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments of the gastric residence systems, where the gastric residence systems have a residence period of about seven days, during the initial six hours of residence after administration the gastric residence systems release about 2% to about 10% of the total mass of agent (or salt thereof) released over the residence period of seven days, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments, during the initial 24 hours of residence after administration, the gastric residence systems release about 10% to about 35% of the total mass of agent (or salt thereof) released over the residence period, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

In some embodiments, where the gastric residence systems have a residence period of about seven days, during the initial 24 hours of residence after administration the gastric residence systems release about 10% to about 35% of the total mass of agent (or salt thereof) released over the residence period of seven days, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

### Elastomers

Elastomers (also referred to as elastic polymers or tensile polymers) enable the gastric residence system to be compacted, such as by being folded or compressed, into a form suitable for administration to the stomach by swallowing a container or capsule containing the compacted system. Upon dissolution of the capsule in the stomach, the gastric residence system expands into a shape which prevents passage of the system through the pyloric sphincter of the patient for the desired residence time of the system. Thus, the elastomer must be capable of being stored in a compacted configuration in a capsule for a reasonable shelf life, and of expanding to its original shape, or approximately its original shape, upon release from the capsule. In one embodiment, the elastomer is a silicone elastomer. In one embodiment, the elastomer is formed from a liquid silicone rubber (LSR), such as sold in the Dow Corning QP-1 liquid silicone rubber kit. In one embodiment, the elastomer is crosslinked polycaprolactone. In one embodiment, the elastomer is an enteric polymer, such as those listed in the Enteric Polymer Table. In some embodiments, the coupling polymer(s) used in the system are also elastomers. Elastomers are also useful as the central polymer in the star-shaped or stellate design of the gastric residence systems.

In one embodiment, both the coupling polymer and elastomer are enteric polymers, which provides for more complete breakage of the system into the carrier polymer-agent pieces if the system enters the intestine, or if the patient drinks a mildly basic solution in order to induce passage of the system.

Examples of elastomers which can be used include silicones, such as those formed using Dow Corning QP-1 kits; urethane-cross-linked polycaprolactones; poly(acryloyl 6-aminocaproic acid) (PA6ACA); poly(methacrylic acid-co-ethyl acrylate) (EUDRAGIT L 100-55); and mixtures of poly(acryloyl 6-aminocaproic acid) (PA6ACA) and poly(methacrylic acid-co-ethyl acrylate) (EUDRAGIT L 100-55).

Flexible coupling polymers, i.e., elastomeric coupling polymers or elastomers, are used as the central polymer in the star-shaped or stellate design of the gastric residence systems. A particularly useful elastomer for use as the central elastomer of the stellate or star configuration is silicone rubber. Liquid silicone rubber (LSR) can be molded easily and cured into a desired shape. The Dow Corning QP-1 series, comprising cross-linked dimethyl and methyl-vinyl siloxane copolymers and reinforcing silica, are examples of such silicone rubber polymers (see, for example, the Web site www.dowcorning.com/DataFiles/090276fe8018ed07.pdf). Non-segmented arms or arms comprising segments of carrier polymer-agent components can then be attached to the central silicone rubber elastomer. Another elastomer which can be used as the central elastomer in the stellate design is crosslinked polycaprolactone.

Specific configurations of gastric residence systems are disclosed in International Patent Application No. WO 2017/100367, and any of those configurations can be used for the gastric residence systems disclosed herein.

### Carrier Polymers for Segments and Arms (Carrier Polymer-Agent Component)

The segments and arms of the gastric residence system comprise a carrier polymer-agent component, which comprises the agent (or a pharmaceutically acceptable salt of an agent) to be eluted from the gastric residence system in the gastric environment. The agent is blended into the carrier polymer to form a carrier polymer-agent mixture. This mixture can be formed into the desired shape or shapes for use as carrier polymer-agent components in the systems. After the drug or drug salt is blended into the carrier polymer to form the carrier polymer-drug mixture, the drug or drug salt is distributed or dispersed throughout the blended mixture. If excipients, anti-oxidants, or other ingredients are included in the carrier polymer-drug blend, they will also be distributed or dispersed throughout the blended mixture.

Selection of the carrier material for the agent or pharmaceutically acceptable salt thereof in a gastric residence system influences the release profile of drug during the period of gastric residence. Carrier polymers may be thermoplastic, to allow extrusion using hot melt extrusion or 3D printing techniques. They may also have a high enough melt strength and viscosity to enable extrusion into the required geometry. They may have low melting temperatures (for example, less than about 120° C.), to avoid exposing agents or drugs to high temperatures during manufacture. They may have a sufficient mechanical strength (Young's modulus, compression strength, tensile strength) to avoid breaking in the stomach during the desired residence period. Further, they should be capable of forming stable blends with agents, therapeutic agents, drugs, excipients, dispersants, and other additives.

Exemplary carrier polymers suitable for use in this invention include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, poly(ethylene-co-vinyl acetate), and copolymers, mixtures, blends and combinations thereof.

Polycaprolactone (PCL) and/or thermoplastic polyurethanes are preferred carrier polymers. In some embodiments, polydioxanone is used as the carrier polymer. In any of the embodiments of the gastric residence system, the carrier polymer used in the gastric residence system can comprise polycaprolactone, such as linear polycaprolactone with a number-average molecular weight (Mn) range between about 60 kiloDalton (kDa) to about 100 kDa; 75 kDa to 85 kDa; or about 80 kDa; or between about 45 kDa to about 55 kDa; or between about 50 kDa to about 110,000 kDa, or between about 80 kDa to about 110,000 kDa.

Further, release of drug can be modulated by a wide variety of excipients included in the carrier polymer-agent component. Soluble excipients include P407, Eudragit E, PEG, Polyvinylpyrrolidone (PVP), and Polyvinyl alcohol (PVA). Insoluble, wicking excipients include Eudragit RS and Eudragit RL. Degradable excipients include PLA, PLGA, PLA-PCL, polydioxanone, and linear copolymers of caprolactone and glycolide; polyaxial block copolymers of glycolide, caprolactone, and trimethylene carbonate; polyaxial block copolymers of glycolide, trimethylene carbonate, and lactide; polyaxial block copolymers of glycolide, trimethylene carbonate and polypropylene succinate; polyaxial block copolymers of caprolactone, lactide, glycolide, and trimethylene carbonate; polyaxial block copolymers of glycolide, trimethylene carbonate, and caprolactone; and linear block copolymers of lactide, caprolactone, and trimethylene carbonate; such as linear copolymers of caprolactone (95%) and glycolide (5%); polyaxial block copolymers of glycolide (68%), caprolactone (29%), and trimethylene carbonate (3%); polyaxial block copolymers of glycolide (86%), trimethylene carbonate (9%), and lactide (5%); polyaxial block copolymers of glycolide (70%), trimethylene carbonate (27%) and polypropylene succinate (2%); polyaxial block copolymers of caprolactone (35%), lactide (34%), glycolide (17%), and trimethylene carbonate (14%); polyaxial block copolymers of glycolide (55%), trimethylene carbonate (25%), and caprolactone (20%); and linear block copolymers of lactide (39%), caprolactone (33%), and trimethylene carbonate (28%). Insoluble, swellable excipients include Polyvinyl acetate (PVAc), Crospovidone, Croscarmellose, HPMCAS, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%). Surfactants include Lecithin, Taurocholate, SDS, Soluplus, Fatty acids, and Kolliphor RH40.

Other excipients can be added to the carrier polymers to modulate the release of agent. Such excipients can be added in amounts from about 1% to 75%, from about 5% to 50%, or from about 5% or about 30%. Examples of such excipients include Poloxamer 407 (available as Kolliphor P407, Sigma Cat #62035), poly(ethylene glycol)-block-poly(propylene glycol)-block-poly(ethylene glycol), CAS No. 9003-11-6; H—(OCH2CH2)x-(O—CH(CH3)CH2)y-(OCH2CH2)z-OH where x and z are about 101 and y is about 56); Pluronic P407; Eudragit E, Eudragit EPO (available from Evonik); hypromellose (available from Sigma, Cat #H3785), Kolliphor RH40 (available from Sigma, Cat #07076), polyvinyl caprolactam, polyvinyl acetate (PVAc), polyvinylpyrrolidone (PVP), polyvinyl alcohol (PVA), polyethylene glycol (PEG), and Soluplus (available from BASF; a copolymer of polyvinyl caprolactam, polyvinyl acetate, and polyethylene glycol). Preferred soluble excipients include Eudragit E, polyethylene glycol (PEG), polyvinylpyrrolidone (PVP), polyvinyl acetate (PVAc), and polyvinyl alcohol (PVA). Preferred insoluble excipients include Eudragit RS and Eudragit RL. Preferred insoluble, swellable excipients include crospovidone, croscarmellose, hypromellose acetate succinate (HPMCAS), and carbopol. EUDRAGIT RS and EUDRAGIT RL are registered trademarks of Evonik (Darmstadt, Germany) for copolymers of ethyl acrylate, methyl methacrylate and methacrylic acid ester with quaternary ammonium groups (trimethylammonioethyl methacrylate chloride), having a molar ratio of ethyl acrylate, methyl methacrylate and trimethylammonioethyl methacrylate of about 1:2:0.2 in Eudragit® RL and about 1:2:0.1 in Eudragit® RS. Preferred insoluble, swellable excipients include crospovidone, croscarmellose, hypromellose acetate succinate (HPMCAS), carbopol, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%).

Further examples of excipients that can be used in the segments of the gastric residence system are listed in the Excipient Table below.

Excipient Table

| Function | General Examples | Specific Examples |
|---|---|---|
| Polymeric and | Polyalkylene oxides | Kolliphor RH, Kolliphor P407, Soluplus, |
| non-polymeric | Polyethoxylated castor oil | Cremophor, SDS |
| solubilizers | Detergents | |
| Release-enhancing | Acrylate polymers | Eudragit RL |
| excipient (porogen | Acrylate co-polymers | Eudragit RS |
| or wicking agent) | Polyvinylpyrrolidone | Eudragit E |
| | | Linear block copolymer of dioxanone and |
| | | ethlyne glycol (e.g., 80:20 ratio) |
| Dispersant | porous inorganic material | silica, hydrophilic-fumed silica, hydrophobic |
| | polar inorganic material | colloidal silica, magnesium aluminum |
| | non-toxic metal oxides | silicate, stearate salts, calcium stearate, |
| | amphiphilic organic | magnesium stearate, microcrystalline |
| | molecules | cellulose, carboxymethylcellulose, |
| | polysaccharides, | hypromellose, phospholipids, |
| | cellulose, cellulose | polyoxyethylene stearates, zinc acetate, |
| | derivatives | alginic acid, lecithin, sodium lauryl sulfate, |
| | fatty acids | aluminum oxide |
| | detergents | |
| Stabilizer/ | Anti-oxidants | Tocopherols |
| Preservative agent | Anti-microbial agents | Alpha-tocopherol |
| | Buffering | Ascorbic acid; ascorbate salts |
| | substances/pH | Carotenes |
| | stabilizers | Butylated hydroxytoluene (BHT) |
| | | Butylated hydroxyanisole (BHA) |
| | | Fumaric acid |
| | | calcium carbonate |
| | | calcium lactate |
| | | calcium phosphate |
| | | sodium phosphate |
| | | sodium bicarbonate |

Carrier Polymer-Agent/Agent Salt Combinations with Excipients and Other Additives

The blend of carrier polymer-agent or carrier polymer-agent salt can comprise various excipients and other additives. The following Table CPE-1 lists combinations of excipients and other additives that can be used in combination with agent or salt thereof and carrier polymer in the compositions making up the arms or segments of arms of the gastric residence systems. These excipients and other additives can be combined with agent or salt thereof (where the agent or agent salt comprises between about 10% to about 60% by weight of the composition) with the carrier polymer, such as polycaprolactone, making up the remainder of the composition. Excipients include the following, which can be used individually or in any combination, in amounts ranging from about 1% to about 30%, such as about 5% to about 20%, by weight of the composition: Kolliphor P407 (poloxamer 407, poly(ethylene glycol)-block-poly(propylene glycol)-block-poly(ethylene glycol)), Eudragit RS (Poly[Ethyl acrylate, methyl methacrylate, trimethylammonioethyl methacrylate chloride]1:2:0.1), Eudragit RL (Poly[Ethyl acrylate, methyl methacrylate, trimethylammonioethyl methacrylate chloride]1:2:0.2), PDO (polydioxanone), PEG-PCL, SIF (FaSSIF/FaSSGF powder from BioRelevant), EPO (dimethylaminoethyl methacrylate-butyl methacrylate-methyl methacrylate copolymer), Kollidon VA64 (vinylpyrrolidone-vinyl acetate copolymer in a ratio of 6:4 by mass), polyvinyl acetate, polyvinyl pyrrolidine.

Other additives include silicon dioxide (comprising, for example, about 0.1% to about 5% by weight of the composition, such as about 0.1% to 1% or about 0.5%) and an anti-oxidant, such as alpha-tocopherol (comprising, for example, about 0.1% to about 5% by weight of the composition, such as about 0.1% to 1% or about 0.5%). Each row of the table below represents a formulation of excipients and other additives for use with the carrier polymer and agent or salt thereof.

TABLE CPE-1
Excipients and additives, in combination with
agent or salt thereof and carrier polymer

EPO, P407, Silica, α-tocopherol
EPO, Silica, α-tocopherol
Eudragit RL, Eudragit RS, Kolliphor P407, Silica, α-tocopherol
Eudragit RL, Kolliphor P407, Silica, α-tocopherol
Eudragit RL, Eudragit RS, Kolliphor P407, Silica, α-tocopherol
Eudragit RL, Kolliphor P407, Silica, α-tocopherol
Eudragit RL, Kolliphor P407, Silica, α-tocopherol
Eudragit RS, P407, Silica, α-tocopherol
Eudragit RS, Silica, α-tocopherol
Kollidon VA64, Silica, α-tocopherol
Kolliphor P407, Silica, α-tocopherol
Kolliphor RH40, Silica, α-tocopherol
PDO, Silica, α-tocopherol
PEG-PCL, Silica, α-tocopherol
Poly Vinyl Acetate, Silica, α-tocopherol
PVP, Silica, α-tocopherol
SIF, Silica, α-tocopherol
Silica, P188, P407, α-tocopherol
Silica, α-tocopherol

Table CPE-2 lists specific amounts of excipients and other additives that can be used in combination with agent or salt thereof and carrier polymer in the compositions making up the arms or segments of arms of the gastric residence systems.

The amounts listed in Table CPE-2 can be varied by plus-or-minus 20% of each ingredient (for example, 0.5% silica can vary between 0.4% and 0.6% silica, as 20% of 0.5% is 0.1%). Each row of the table below represents a formulation of excipients and other additives for use with the carrier polymer and agent or salt thereof.

TABLE CPE-2
Excipients and additives, in combination with
agent or salt thereof and carrier polymer

0.5% Silica, 0.5% α-tocopherol
0.5% Silica, 2% P407, 0.5% α-tocopherol
0.5% Silica, 2% P188, 2% P407, 0.5% α-tocopherol
0.5% Silica, 3% Eudragit RS, 2% P407, 0.5% α-tocopherol
1% Kolliphor P407, 0.5% Silica, 0.5% α-tocopherol
10% Eudragit RS, 2.5% P407, 2% Silica, 0.5% α-tocopherol
10% Eudragit RS, 5% P407, 0.5% Silica, 0.5% α-tocopherol
10% Eudragit RS, 5% P407, 2% Silica, 0.5% α-tocopherol
12% Eudragit RL, 3% Kolliphor P407, 0.5% Silica, 0.5% α-tocopherol
12% Eudragit RL, 5% Kolliphor P407, 0.5% Silica, 0.5% α-tocopherol
14.78% Eudragit RS, 0.226% P407, 0.5% Silica, 0.5% α-tocopherol
17.5% Eudragit RS, 5% P407, 0.5% Silica, 0.5% α-tocopherol
19.8% Eudragit RS, 0.5% Silica, 0.5% α-tocopherol
2% Kolliphor P407, 0.5% Silica, 0.5% α-tocopherol
2% P407, 0.5% Silica, 0.5% α-tocopherol
20% Eudragit RS, 2% P407, 0.5% Silica, 0.5% α-tocopherol
21.25% Eudragit RS, 2.5% P407, 0.5% Silica, 0.5% α-tocopherol
25% Eudragit RL, 5% P407, 0.5% Silica, 0.5% α-tocopherol
25% Eudragit RS, 0.5% Silica, 0.5% α-tocopherol
25% Eudragit RS, 5% P407, 0.5% Silica, 0.5% α-tocopherol
3% Eudragit RL, 9% Eudragit RS, 5% Kolliphor P407, 0.5% Silica,
0.5% α-tocopherol
3.5% Eudragit RS, 2.5% P407, 2% Silica, 0.5% α-tocopherol
3.5% Eudragit RS, 5% P407, 2% Silica, 0.5% α-tocopherol
30% PDO, 0.5% Silica, 0.5% α-tocopherol
39.5% PEG-PCL, 0.36% Silica, 0.36% α-tocopherol
4.5% EPO, 4.5% P407, 0.5% Silica, 0.5% α-tocopherol
5% Kolliphor P407, 0.5% Silica, 0.5% α-tocopherol
5% Kolliphor RH40, 0.5% Silica, 0.5% α-tocopherol
5% SIF, 0.5% Silica, 0.5% α-tocopherol
6% Eudragit RL, 5% Kolliphor P407, 0.5% Silica, 0.5% α-tocopherol
6% Eudragit RL, 6% Eudragit RS, 5% Kolliphor P407, 0.5% Silica,
0.5% α-tocopherol
6.75% Eudragit RS, 3.75% P407, 2% Silica, 0.5% α-tocopherol
7% EPO, 2% P407, 0.5% Silica, 0.5% α-tocopherol
9% EPO, 0.5% Silica, 0.5% α-tocopherol
9% Eudragit RL, 3% Eudragit RS, 5% Kolliphor P407, 0.5% Silica,
0.5% α-tocopherol
9% Kollidon VA64, 0.5% Silica, 0.5% α-tocopherol
9% Poly Vinyl Acetate, 0.5% Silica, 0.5% α-tocopherol
9% PVP, 0.5% Silica, 0.5% α-tocopherol
9% SIF, 0.5% Silica, 0.5% α-tocopherol

### Agents for Use in Gastric Residence Systems

Agents (active pharmaceutical ingredients, such as therapeutic agents) which can be administered to or via the gastrointestinal tract can be used in the gastric residence systems of the invention. The agent is blended with the carrier polymer, and any other excipients or other additives to the carrier polymer, and formed into a segment for use in a gastric residence system. Agents include, but are not limited to, drugs, pro-drugs, biologics, and any other substance which can be administered to produce a beneficial effect on an illness or injury.

Agents that can be used in the gastric residence systems of the invention include statins, such as rosuvastatin; nonsteroidal anti-inflammatory drugs (NSAIDs) such as meloxicam; selective serotonin reuptake inhibitors (SSRIs) such as escitalopram and citalopram; blood thinners, such as clopidogrel; steroids, such as prednisone; antipsychotics, such as aripiprazole and risperidone; analgesics, such as buprenorphine; opioid antagonists, such as naloxone; anti-asthmatics such as montelukast; anti-dementia drugs, such as memantine; cardiac glycosides such as digoxin; alpha blockers such as tamsulosin; cholesterol absorption inhibitors such as ezetimibe; anti-gout treatments, such as colchicine; antihistamines, such as loratadine and cetirizine, opioids, such as loperamide; proton-pump inhibitors, such as omeprazole; antiviral agents, such as entecavir; antibiotics, such as doxycycline, ciprofloxacin, and azithromycin; antimalarial agents; levothyroxine; substance abuse treatments, such as methadone and varenicline; contraceptives; stimulants, such as caffeine; and nutrients such as folic acid, calcium, iodine, iron, zinc, thiamine, niacin, vitamin C, vitamin D, biotin, plant extracts, phytohormones, and other vitamins or minerals. Biologics that can be used as agents in the gastric residence systems of the invention include proteins, polypeptides, polynucleotides, and hormones. Exemplary classes of agents include, but are not limited to, analgesics; anti-analgesics; anti-inflammatory drugs; antipyretics; antidepressants; antiepileptics; antipsychotic agents; neuroprotective agents; anti-proliferatives, such as anti-cancer agents; antihistamines; antimigraine drugs; hormones; prostaglandins; antimicrobials, such as antibiotics, antifungals, antivirals, and antiparasitics; anti-muscarinics; anxiolytics; bacteriostatics; immunosuppressant agents; sedatives; hypnotics; antipsychotics; bronchodilators; anti-asthma drugs; cardiovascular drugs; anesthetics; anti-coagulants; enzyme inhibitors; steroidal agents; steroidal or non-steroidal anti-inflammatory agents; corticosteroids; dopaminergics; electrolytes; gastro-intestinal drugs; muscle relaxants; nutritional agents; vitamins; parasympathomimetics; stimulants; anorectics; anti-narcoleptics; and antimalarial drugs, such as quinine, lumefantrine, chloroquine, amodiaquine, pyrimethamine, proguanil, chlorproguanil-dapsone, sulfonamides (such as sulfadoxine and sulfamethoxypyridazine), mefloquine, atovaquone, primaquine, halofantrine, doxycycline, clindamycin, artemisinin, and artemisinin derivatives (such as artemether, dihydroartemisinin, arteether and artesunate). The term “agent” includes salts, solvates, polymorphs, and co-crystals of the aforementioned substances. In some embodiments, the agent is selected from the group consisting of cetirizine, rosuvastatin, escitalopram, citalopram, risperidone, olanzapine, donepezil, and ivermectin. In some embodiments, the agent is one that is used to treat a neuropsychiatric disorder, such as an anti-psychotic agent such as risperidone.

Agents can be used in the gastric residence systems of the invention in any suitable crystalline form, or in amorphous form, or in both crystalline form or forms and amorphous forms. That is, agent or drug particles contained in the gastric residence systems can be used in crystalline form, in amorphous form, or in a mixture of crystalline forms (either a single crystalline form, or multiple crystalline forms) and amorphous forms, so as to provide a desired rate of release or desired physical or chemical properties.

Gastric residence systems are well-suited for use in treatment of diseases and disorders which present difficulties with patient compliance, and thus in some embodiments, the gastric residence systems are used to treat a disease or disorder where patient compliance with a medication regimen is problematic. Such diseases and disorders include neuropsychiatric diseases and disorders, dementia and other diseases and disorders which affect memory, Alzheimer's disease, psychoses, schizophrenia, and paranoia. Accordingly, agents which can be used in the gastric residence systems include, but are not limited to, anti-dementia agents, anti-Alzheimer's disease agents, and anti-psychotics.

Exemplary hydrophilic agents which can be used in the systems include risperidone, cetirizine, memantine, and olanzapine. Exemplary hydrophobic agents which can be used in the systems include aripiprazole, ivermectin, rosuvastatin, citalopram, and escitalopram.

In some embodiments, the agent or salt thereof (for example, a drug) makes up about 10% to about 40% by weight of the arm or segment, and thus the carrier polymer and any other components of the arm or segment blended into the carrier polymer together make up the remainder of the weight of the arm or segment. In some embodiments, the agent or salt thereof makes up about 10% to about 35%, about 10% to about 30%, about 10% to about 25%, about 10% to about 20%, about 10% to about 15%, about 15% to about 40%, about 20% to about 40%, about 25% to about 40%, about 30% to about 40%, about 35% to about 40%, about 15% to about 35%, about 20% to about 35%, or about 25% to about 40% by weight of the arm or segment.

Further embodiments of arms or segments, where the agent or salt thereof makes up more than about 40% by weight of the arm or segment, are described below under “high agent loading of arms and segments.”

### Agent Loading of Arms and Segments

The agent or active pharmaceutical ingredient, or pharmaceutically acceptable salt thereof, can be loaded into an arm or into a segment of an arm in an amount ranging from about 10% to about 80% by weight of the arm or of the segment of the arm. Agent/API loading can be about 10% to about 70%, about 10% to about 60%, about 10% to about 50%, about 10% to about 40%, about 10% to about 30%, about 10% to about 20%, about 20% to about 80%, about 30% to about 80%, about 40% to about 80%, about 50% to about 80%, about 60% to about 80%, about 70% to about 80%, about 30% to about 70%, or about 40% to about 60% by weight of the arm or of the segment of the arm.

In some embodiments of the invention, the arms, or segments of which the arms are comprised, can have a high loading of agent or pharmaceutically acceptable salt thereof. “High loading” generally refers to arms or arm segments where the agent or salt thereof (for example, a drug) makes up between about 40% by weight of the arm or segment, to about 80% by weight of the arm or segment. Any components of the arms or arm segments which are not blended into the carrier polymer are not included in the calculation of the weight percentage; for example, if an arm has one or more disintegrating matrices interspersed between segments of the arm, the weight of such matrices would not be included as part of the weight of the arm in the calculation of the weight percentage of agent in the arm.

In some embodiments, the amount of agent by weight in the arms, or segments of which the arms are comprised, can comprise at least about 40%, at least about 45%, at least about 50%, at least about 55%, or about 60%. In some embodiments, the amount of agent by weight in the arms, or segments of which the arms are comprised, can comprise about 40% to about 60%, about 45% to about 60%, about 50% to about 60%, about 55% to about 60%, about 40% to about 55%, about 40% to about 50%, or about 40% to about 45%. In some embodiments, the amount of agent by weight in the arms, or segments of which the arms are comprised, can comprise about 25% to about 60%, about 30% to about 60%, or about 35% to about 60%. In some embodiments, the amount of agent by weight in the arms, or segments of which the arms are comprised, can comprise about 51% to about 60%, about 52% to about 60%, about 53% to about 60%, about 54% to about 60%, about 55% to about 60%, about 56% to about 60%, or about 57% to about 60%.

The combination of the high agent or agent salt loading with the release rate-controlling polymer film provides gastric residence systems with increased amounts of agent or agent salt, while maintaining good release kinetics over the residence period of the system.

### Dispersants for Modulation of Agent Release and Stability of Polymer Blend

The use of a dispersant in the carrier polymer-agent component provides numerous advantages. The rate of elution of agent from the carrier polymer-agent component is affected by numerous factors as previously noted, including the composition and properties of the carrier polymer (which may itself comprise multiple polymeric and non-polymeric components); the physical and chemical properties of the agent; and the gastric environment. Avoiding burst release of agent, especially hydrophilic agents, and maintaining sustained release of the agent over the effective release period or residence period is an important characteristic of the systems. The use of a dispersant according to the invention enables better control of release rate and suppression of burst release. Burst release and release rate can be tuned by using varied concentrations of dispersant. For example, different dispersants and different excipients, at varying concentrations, can tune burst release of cetirizine in simulated gastric fluid.

Dispersants which can be used in the invention include: silicon dioxide (silica, SiO2) (hydrophilic fumed); stearate salts, such as calcium stearate and magnesium stearate; microcrystalline cellulose; carboxymethylcellulose; hydrophobic colloidal silica; hypromellose; magnesium aluminum silicate; phospholipids; polyoxyethylene stearates; zinc acetate; alginic acid; lecithin; fatty acids; sodium lauryl sulfate; and non-toxic metal oxides such as aluminum oxide. Porous inorganic materials and polar inorganic materials can be used. Hydrophilic-fumed silicon dioxide is a preferred dispersant. One particularly useful silicon dioxide is sold by Cabot Corporation (Boston, Mass., USA) under the registered trademark CAB-O-SIL® M-5P (CAS #112945-52-5), which is hydrophilic-fumed silicon dioxide having a BET surface area of about 200 m2/g±15 m2/g. The mesh residue for this product on a 45 micron sieve is less than about 0.02%. The typical primary aggregate size is about 150 to about 300 nm, while individual particle sizes may range from about 5 nm to about 50 nm.

In addition to anti-aggregation/anti-flocculation activity, the dispersant can help prevent phase separation during fabrication and/or storage of the systems. This is particularly useful for manufacture of the systems by hot melt extrusion.

The weight/weight ratio of dispersant to agent substance can be about 0.1% to about 5%, about 0.1% to about 4%, about 0.1% to about 3%, about 0.1% to about 2%, about 0.1% to about 1%, about 1% to about 5%, about 1% to about 4%, about 1% to about 3%, about 1% to about 2%, about 2% to about 4%, about 2% to about 3%, about 3% to about 4%, about 4% to about 5%, or about 0.1%, about 0.5%, about 1%, about 2%, about 3%, about 4% or about 5%.

Dispersants can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Dispersants can also be used to modulate the amount of burst release of agent or pharmaceutically acceptable salt thereof during the initial period when the gastric residence system is administered. In embodiments of a gastric residence system that is to be administered once weekly, the burst release over the approximately first six hours after initial administration is less than about 8%, preferably less than about 6%, of the total amount of agent (or salt thereof) in the system. In embodiments of a gastric residence system that is to be administered once every three days, the burst release over the approximately first six hours after initial administration is less than about 12%, preferably less than about 10%, of the total amount of agent (or salt thereof) in the system. In embodiments of a gastric residence system that is to be administered once daily, the burst release over the approximately first six hours after initial administration is less than about 40%, preferably less than about 30%, of the total amount of agent (or salt thereof) in the system. In general, if a new gastric residence system is administered every D days, and the total mass of agent (or salt thereof) is M, then the gastric residence system releases less than about [(M divided by D) times 0.5], preferably less than about [(M divided by D) multiplied by 0.4], or less than about [(M divided by D) multiplied by %], more preferably less than about [(M divided by D) multiplied by 0.3], over the approximately first six hours after initial administration. In further embodiments, the gastric residence system releases at least about [(M divided by D) multiplied by 0.25] over the approximately first six hours after initial administration, that is, the system releases at least about one-quarter of the daily dosage over the first one-quarter of the first day of administration.

### Stabilizers for Use in Gastric Residence Systems

Many agents are prone to oxidative degradation when exposed to reactive oxygen species, which can be present in the stomach. An agent contained in the system may thus oxidize due to the prolonged residence in the stomach of the system, and the extended release period of agent from the system. Accordingly, it is desirable to include stabilizers or preservatives in the systems, in order to stabilize the agent to prevent oxidative and other degradation.

Stabilizers, such as anti-oxidants including tocopherols, alpha-tocopherol, ascorbic acid, ascorbyl palmitate, butylated hydroxytoluene, butylated hydroxyanisole, and fumaric acid, can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Anti-oxidant stabilizers that can be included in the systems to reduce or prevent oxidation of the agent include alpha-tocopherol (about 0.01 to about 0.05% v/v), ascorbic acid (about 0.01 to about 0.1% w/v), ascorbyl palmitate (about 0.01 to about 0.1% w/v), butylated hydroxytoluene (about 0.01 to about 0.1% w/w), butylated hydroxyanisole (about 0.01 to about 0.1% w/w), and fumaric acid (up to 3600 ppm). Vitamin E, a tocopherol, a Vitamin E ester, a tocopherol ester, ascorbic acid, or a carotene, such as alpha-tocopherol, Vitamin E succinate, alpha-tocopherol succinate, Vitamin E acetate, alpha-tocopherol acetate, Vitamin E nicotinate, alpha-tocopherol nicotinate, Vitamin E linoleate, or alpha-tocopherol linoleate can be used as anti-oxidant stabilizers.

Certain agents can be pH-sensitive, especially at the low pH present in the gastric environment. Buffering or pH-stabilizer compounds that can be included in the systems to reduce or prevent degradation of agent at low pH include calcium carbonate, calcium lactate, calcium phosphate, sodium phosphate, and sodium bicarbonate. They are typically used in an amount of up to about 2% w/w. The buffering or pH-stabilizer compounds can comprise about 0.1% to about 4% of the carrier polymer-agent components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

The anti-oxidant stabilizers, pH stabilizers, and other stabilizer compounds are blended into the polymers containing the agent (or pharmaceutically acceptable salt thereof) by blending the stabilizer(s) into the molten carrier polymer-agent or agent salt mixture. The stabilizer(s) can be blended into molten carrier polymer prior to blending the agent (or salt thereof) into the polymer-stabilizer mixture; or the stabilizer(s) can be blended with agent (or salt thereof) prior to formulation of the blended agent (or salt thereof)-stabilizer mixture in the carrier polymer; or stabilizer(s), agent (or salt thereof), and molten carrier polymer can be blended simultaneously. Agent (or salt thereof) can also be blended with molten carrier polymer prior to blending the stabilizer(s) into the polymer-agent or agent salt mixture.

In one embodiment, less than about 10% of the agent (or salt thereof) remaining in the system is degraded or oxidized after a gastric residence period of about 24 hours. In one embodiment, less than about 10% of the agent (or salt thereof) remaining in the system is degraded or oxidized after a gastric residence period of about 48 hours. In one embodiment, less than about 10% of the agent (or salt thereof) remaining in the system is degraded or oxidized after a gastric residence period of about 72 hours. In one embodiment, less than about 10% of the agent (or salt thereof) remaining in the system is degraded or oxidized after a gastric residence period of about 96 hours. In one embodiment, less than about 10% of the agent (or salt thereof) remaining in the system is degraded or oxidized after a gastric residence period of about five days. In some embodiments, less than about 10% of the agent (or salt thereof) remaining in the system is degraded or oxidized after a gastric residence period of about a week. In some embodiments, less than about 10% of the agent (or salt thereof) remaining in the system is degraded or oxidized after a gastric residence period of about two weeks.

In one embodiment, less than about 5% of the agent (or salt thereof) remaining in the system is degraded or oxidized after a gastric residence period of about 24 hours. In one embodiment, less than about 5% of the agent (or salt thereof) remaining in the system is degraded or oxidized after a gastric residence period of about 48 hours. In one embodiment, less than about 5% of the agent (or salt thereof) remaining in the system is degraded or oxidized after a gastric residence period of about 72 hours. In one embodiment, less than about 5% of the agent (or salt thereof) remaining in the system is degraded or oxidized after a gastric residence period of about 96 hours. In one embodiment, less than about 5% of the agent (or salt thereof) remaining in the system is degraded or oxidized after a gastric residence period of about five days. In some embodiments, less than about 5% of the agent (or salt thereof) remaining in the system is degraded or oxidized after a gastric residence period of about a week. In some embodiments, less than about 5% of the agent (or salt thereof) remaining in the system is degraded or oxidized after a gastric residence period of about two weeks.

### Coupling Polymers

The coupling polymer is used to link one or more carrier polymer-agent components (i.e., arm or segment of an arm) to one or more carrier polymer-agent components, to link one or more carrier polymer-agent components to one or more elastomer components (i.e., core), or to link one or more elastomer components to one or more elastomer components. Thus, the coupling polymers form linker regions between other components of the system. Enteric polymers and time-dependent polymers are preferred for use as coupling polymers. In some embodiments, enteric polymers are used as coupling polymers. In some embodiments, time-dependent polymers which are pH-resistant, that is, less sensitive to changes in pH than enteric polymers, are used as coupling polymers. In some embodiments, both enteric polymers and time-dependent polymers which are less sensitive to changes in pH than enteric polymers are used as coupling polymers.

Enteric polymers are relatively insoluble under acidic conditions, such as the conditions encountered in the stomach, but are soluble under the less acidic to basic conditions encountered in the small intestine. Enteric polymers which dissolve at about pH 5 or above can be used as coupling polymers, as the pH of the initial segment of the small intestine, the duodenum, ranges from about 5.4 to 6.1. If the gastric residence system passes intact through the pyloric valve, the enteric coupling polymer will dissolve and the components linked by the coupling polymer will break apart, allowing passage of the residence system through the small and large intestines. Thus, the gastric residence systems are designed to uncouple rapidly in the intestinal environment by dissolution of the coupling polymer.

By “time-dependent polymer which are pH-resistant” (or equivalently, “pH-resistant time-dependent polymers”) is meant that, under conditions where an enteric polymer would degrade to the point that it would no longer link the components together, the time-dependent polymer will still have sufficient mechanical strength to link the components together. In some embodiments, the time-dependent polymer retains about the same linking capacity, that is, about 100% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 90% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 75% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 60% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 50% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 25% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer resists breaking under a flexural force of about 0.2 Newtons (N), about 0.3 N, about 0.4 N, about 0.5 N, about 0.75 N, about 1 N, about 1.5 N, about 2 N, about 2.5 N, about 3 N, about 4 N, or about 5 N, after exposure to a solution between about pH 7 to about pH 8, where the exposure is for about an hour, about a day, about three days, or about a week. Linkage strength can be measured by any relevant test that serves to test coupling ability, such as a four-point bending flexural test (ASTM D6272) or a modified test based on the four-point bending flexural test.

Exemplary coupling polymers include, but are not limited to, cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methacrylic acid methylmethacrylate copolymer, methyl acrylate-methacrylic acid copolymer, methacrylate-methacrylic acid-octyl acrylate copolymer, and copolymers, mixtures, blends and combinations thereof. Some of the enteric polymers that can be used in the invention are listed in the Enteric Polymer Table, along with their dissolution pH. (See Mukherji, Gour and Clive G. Wilson, “Enteric Coating for Colonic Delivery,” Chapter 18 of Modified-Release Drug Delivery Technology (editors Michael J. Rathbone, Jonathan Hadgraft, Michael S. Roberts), Drugs and the Pharmaceutical Sciences Volume 126, New York: Marcel Dekker, 2002.) Preferably, enteric polymers that dissolve at a pH of no greater than about 5 or about 5.5 are used. Poly(methacrylic acid-co-ethyl acrylate) (sold under the trade name EUDRAGIT L 100-55; EUDRAGIT is a registered trademark of Evonik Rohm GmbH, Darmstadt, Germany) is a preferred enteric polymer. Another preferred enteric polymer is hydroxypropylmethylcellulose acetate succinate (hypromellose acetate succinate or HPMCAS; Ashland, Inc., Covington, Ky., USA), which has a tunable pH cutoff from about 5.5 to about 7.0. Cellulose acetate phthalate, cellulose acetate succinate, and hydroxypropyl methylcellulose phthalate are also suitable enteric polymers.

In one embodiment, the enteric polymers used in the gastric residence system dissolve at a pH above about 4. In some embodiments, the enteric polymers used in the gastric residence system dissolve at a pH above about 5. In some embodiments, the enteric polymers used in the gastric residence system dissolve at a pH above about 6. In some embodiments, the enteric polymers used in the gastric residence system dissolve at a pH above about 7. In some embodiments, the enteric polymers used in the gastric residence system dissolve at a pH above about 7.5. In some embodiments, the enteric polymers used in the gastric residence system dissolve at a pH between about 4 and about 5. In some embodiments, the enteric polymers used in the gastric residence system dissolve at a pH between about 4 and about 6. In some embodiments, the enteric polymers used in the gastric residence system dissolve at a pH between about 4 and about 7. In some embodiments, the enteric polymers used in the gastric residence system dissolve at a pH between about 4 and about 7.5. In some embodiments, the enteric polymers used in the gastric residence system dissolve at a pH between about 5 and about 6. In some embodiments, the enteric polymers used in the gastric residence system dissolve at a pH between about 5 and about 7. In some embodiments, the enteric polymers used in the gastric residence system dissolve at a pH between about 5 and about 7.5. In some embodiments, the enteric polymers used in the gastric residence system dissolve at a pH between about 6 and about 7. In some embodiments, the enteric polymers used in the gastric residence system dissolve at a pH between about 6 and about 7.5.

Enteric Polymer Table

| | Dissolution |
|---|---|
| Polymer | pH |
| Cellulose acetate phthalate | 6.0-6.4 |
| Hydroxypropyl methylcellulose | 4.8 |
| phthalate 50 | |
| Hydroxypropyl methylcellulose | 5.2 |
| phthalate 55 | |
| Polyvinylacetate phthalate | 5.0 |
| Methacrylic acid-methyl methacrylate | 6.0 |
| copolymer (1:1) | |
| Methacrylic acid-methyl methacrylate | 6.5-7.5 |
| copolymer (2:1) | |
| Methacrylic acid-ethyl acrylate | 5.5 |
| copolymer (2:1) | |
| Shellac | 7.0 |
| Hydroxypropyl methylcellulose | 7.0 |
| acetate succinate | |
| Poly (methyl vinyl ether/maleic acid) | 4.5-5.0 |
| monoethyl ester | |
| Poly (methyl vinyl ether/maleic acid) | 5.4 |
| n-butyl ester | |

Additional polymers useful as coupling polymers are time-dependent polymers, that is, polymers that degrade in a time-dependent manner in the gastric environment. For example, the liquid plasticizer triacetin releases from a polymer formulation in a time-dependent manner over seven days in simulated gastric fluid, while Plastoid B retains its strength over a seven-day period in simulated gastric fluid. Thus, a polymer that degrades in a time-dependent manner can be readily prepared by mixing Plastoid B and triacetin; the degradation time of the Plastoid B-triacetin mixture can be extended by increasing the amount of Plastoid B used in the mixture (that is, using less triacetin in the mixture), while the degradation time can be decreased by decreasing the amount of Plastoid B used in the mixture (that is, using more triacetin in the mixture).

A variety of time-dependent mechanisms are available. Water-soluble time-dependent polymers break down as water penetrates through the polymer. Examples of such polymers are hydroxypropyl methylcellulose and poly vinyl acetate. Acid soluble time-dependent polymers break down over time in an acidic environment. Examples include Eudragit EPO. Time-dependent polymers can use water soluble plasticizers; as plasticizer is released, the remaining polymer becomes brittle and breaks under gastric forces. Examples of such polymers include triacetin and triethyl citrate.

In some embodiments, the carrier polymer-agent components are arms comprised of segments attached by enteric polymers. In some embodiments, the carrier polymer-agent components are attached to the elastomer component of the system by enteric polymers. In any of these embodiments, when enteric polymers are used for both segment-to-segment attachments and for attachment of the arms to the elastomeric component, the enteric polymer used for segment-segment attachments can be the same enteric polymer as the enteric polymer used for attachment of the arms to the elastomeric component, or the enteric polymer used for segment-segment attachments can be a different enteric polymer than the enteric polymer used for attachment of the arms to the elastomeric component. The enteric polymers used for the segment-segment attachments can all be the same enteric polymer, or can all be different enteric polymers, or some enteric polymers in the segment-segment attachments can be the same and some enteric polymers in the segment-segment attachments can be different. That is, the enteric polymer(s) used for each segment-segment attachment and the enteric polymer used for attachment of the arms to the elastomeric component can be independently chosen.

In some embodiments, the carrier polymer-drug components are non-segmented arms attached to the elastomer component of the system by enteric polymers, time-dependent linkers, or disintegrating matrices, or by any combination of enteric polymers, time-dependent linkers, and/or disintegrating matrices.

In any of the embodiments of the gastric residence systems described herein, the coupling polymers or linkers can comprise hydroxypropyl methyl cellulose acetate succinate (HPMCAS) and polycaprolactone (PCL). These blends can be used to form disintegrating linkers or disintegrating matrices. The ratio of HPMCAS to polycaprolactone in the disintegrating linker or disintegrating matrix can be between about 80% HPMCAS:20% PCL to about 20% HPMCAS:80% PCL. The ratio of HPMCAS to polycaprolactone can be between about 80% HPMCAS:20% PCL to about 20% HPMCAS:80% PCL; between about 70% HPMCAS:30% PCL to about 30% HPMCAS:70% PCL; between about 60% HPMCAS:40% PCL to about 40% HPMCAS:60% PCL; between about 80% HPMCAS:20% PCL to about 50% HPMCAS:50% PCL; between about 80% HPMCAS:20% PCL to about 60% HPMCAS:40% PCL; between about 70% HPMCAS:30% PCL to about 50% HPMCAS:50% PCL; between about 70% HPMCAS:30% PCL to about 60% HPMCAS:40% PCL; between about 20% HPMCAS:80% PCL to about 40% HPMCAS:60% PCL; between about 20% HPMCAS:80% PCL to about 50% HPMCAS:50% PCL; between about 30% HPMCAS:70% PCL to about 40% HPMCAS:60% PCL; between about 30% HPMCAS:70% PCL to about 50% HPMCAS:50% PCL; or about 80% HPMCAS:20% PCL, about 70% HPMCAS:30% PCL, about 60% HPMCAS:40% PCL, about 50% HPMCAS:50% PCL, about 40% HPMCAS:60% PCL, about 30% HPMCAS:70% PCL, or about 20% HPMCAS:80% PCL. The linker can further comprise a plasticizer selected from the group consisting of triacetin, triethyl citrate, tributyl citrate, poloxamers, polyethylene glycol, polypropylene glycol, diethyl phthalate, dibutyl sebacate, glycerin, castor oil, acetyl triethyl citrate, acetyl tributyl citrate, polyethylene glycol monomethyl ether, sorbitol, sorbitan, a sorbitol-sorbitan mixture, and diacetylated monoglycerides.

The linkers are chosen to weaken sufficiently after a specified period of time in order to allow the gastric residence systems to reach a point where they de-couple and pass through the pylorus and out of the stomach after the desired residence period or weaken sufficiently such that the gastric residence system is no longer retained in the stomach; that is, the linkers weaken to the point of uncoupling (the uncoupling point) or to the point where the gastric residence system can pass through the pylorus (the pyloric passage point, or passage point). Thus, in one embodiment, linkers are used that uncouple after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In one embodiment, linkers are used that uncouple after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In one embodiment, linkers are used that uncouple after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In one embodiment, linkers are used that uncouple after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In one embodiment, linkers are used that uncouple after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In one embodiment, linkers are used that uncouple after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In one embodiment, linkers are used that uncouple after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

The de-coupling or pyloric passage point in human, dog, or pig occurs when the system passes out of the stomach, that is, when it passes through the pylorus. For the in vitro measurements in simulated gastric fluid or acidic water, the de-coupling or pyloric passage point occurs when the linker weakens to the point where it will break under the normal compressive forces of the stomach, typically about 0.1 Newton to 0.2 Newton. Linkage strength (breaking point) can be measured by any relevant test that serves to test coupling ability, that is, the force required to break the linker, such as the four-point bending flexural test (ASTM D6272) described in Example 18 of WO 2017/070612, or Examples 12, 13, 15, 17, or 18 of WO 2017/100367. In one embodiment, the de-coupling or pyloric passage point is reached when the linkers uncouple at about 0.2 N of force. In another embodiment, the de-coupling or pyloric passage point is reached when the linkers uncouple at about 0.1 N of force.

The gastric residence systems can reach the pyloric passage point without any or all of the linkers actually breaking. If the linkers weaken or degrade to the point where they can no longer hold the gastric residence system in the stomach, even if one, some, or all of the linkers do not break, the gastric residence system will pass through the pylorus and into the small intestine (the pyloric passage point or passage point). In some embodiments, linkers are used that weaken to the passage point after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In some embodiments, linkers are used that weaken to the passage point after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In some embodiments, linkers are used that weaken to the passage point after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In some embodiments, linkers are used that weaken to the passage point after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In some embodiments, linkers are used that weaken to the passage point after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In some embodiments, linkers are used that weaken to the passage point after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In some embodiments, linkers are used that weaken to the passage point after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

### Gastric Residence Systems Comprising Arms of Controlled Stiffness

In some embodiments, gastric residence systems described herein may additionally comprise arms of a controlled thickness to help prevent premature passage of the gastric residence system through a patient's pylorus.

By controlling the stiffness of an element of a gastric residence system that widens/enlarges the device to its open configuration (such as an arm), the risk of premature passage of the gastric residence system through the pylorus may be minimized. Accordingly, gastric residence systems having arms of controlled stiffness can help improve the efficacy and reliability of gastric residence systems. Additionally, gastric residence systems having arms of controlled stiffness can help prevent the gastric residence system from bending into configurations that allow for premature passage through the pylorus.

Gastric residence systems having arms of controlled stiffness require more force for the gastric residence system to bend into configurations suitable for premature passage through the pylorus. Described are gastric residence systems having controlled stiffness of any member that can widen or enlarge the gastric residence system into its open configuration (such as an arm) to help minimize the risk of the gastric residence system passing through the pylorus of a patient prematurely.

A gastric residence system having arms of a controlled stiffness is defined as a system comprising one or more arms having at least a portion of the arm made of a flexible material. In some embodiments, one or more arms may include a first segment comprising a first polymer composition and a second segment comprising a second polymer composition, wherein the second segment is more flexible than the first segment.

In some embodiments, the one or more arms extend radially. A proximal end of the one or more arms may be connected to a core. In some embodiments, a gastric residence system may include a plurality of arms extending radially. In some embodiments, a gastric residence system may include a plurality of arms connected to a core at the proximal end of each arm, the plurality of arms extending radially from the core. In some embodiments, a gastric residence system may comprise a plurality of arms, each arm comprising a first segment and a second segment.

The first polymer composition of a flexible arm of a gastric residence system may comprise a relatively stiff polymer. For example, suitable polymers may include polycaprolactone, polylactic acid, poly(lactic-co-glycolic acid), HPMCAS, high durometer TPU, and/or combinations thereof. Other examples may include hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof. In some embodiments, the first segment may also comprise one or more therapeutic agent or active pharmaceutical ingredients (APIs).

In some embodiments, the first polymer composition may comprise 10-90 wt. % or 50-70 wt. % polycaprolactone. In some embodiments, the first polymer composition may comprise less than 90 wt. %, less than 80 wt. %, less than 70 wt. %, less than 60 wt. %, less than 50 wt. %, less than 40 wt. %, less than 30 wt. %, or less than 20 wt. % polycaprolactone. In some embodiments, the first polymer composition may include more than 20 wt. %, more than 30 wt. %, more than 40 wt. %, more than 50 wt. %, more than 60 wt. %, more than 70 wt. %, or more than 80 wt. % polycaprolactone.

In some embodiments, the first polymer composition may comprise 10-90 wt. % or 30-70 wt. % agent or API. In some embodiments, the first polymer composition may comprise less than 90 wt. %, less than 80 wt. %, less than 70 wt. %, less than 60 wt. %, less than 50 wt. %, less than 40 wt. %, less than 30 wt. %, or less than 20 wt. % agent or API. In some embodiments, the first polymer composition may include more than 20 wt. %, more than 30 wt. %, more than 40 wt. %, more than 50 wt. %, more than 60 wt. %, more than 70 wt. %, or more than 80 wt. % agent or API.

The second polymer composition of an arm of a gastric residence system disclosed herein may comprise a primary polymer that is flexible relative to the polymer of the first polymer composition. For example, suitable relatively “flexible” polymers may include one or more of a polyurethane, a polyether-polyamide copolymer, a thermoplastic elastomer, a thermoplastic polyurethane, polycaprolactone polylactic acid copolymer, a poly(trimethylene carbonate), a polyglycerol sebacate, a polyethylene-co-vinyl acetate, and a silicone. In some embodiments, the second polymer composition of an arm may actually comprise the same primary polymer as the first polymer composition. For example, the second polymer composition may comprise polycaprolactone. However, unlike the first polymer composition, the second polymer composition may additionally comprise a soluble material (e.g., copovidone, poloxamers). Thus, upon hydration (e.g., within the stomach), the second polymer composition will soften such that the stiffness of the second polymer composition of the second segment is less than the first polymer composition of the first segment. Suitable commercially-available polymers may include Pathway™ TPU polymers (The Lubrizol Corporation), Tecoflex™ (The Lubrizol Corporation), Tecophilic™ (The Lubrizol Corporation), Carbothane™ (The Lubrizol Corporation), Isoplast® (The Lubrizol Corporation), Pebax® (Arkema), Texin® (Covestro), Chronoflex (AdvanSource Biomaterials), NEUSoft™ (PolyOne), and Medalist® TPEs (Teknor Apex).

In some embodiments, the second polymer composition may comprise 10-90 wt. % or 40-70 wt. % primary polymer. In some embodiments, the second polymer composition may comprise less than 90 wt. %, less than 80 wt. %, less than 70 wt. %, less than 60 wt. %, less than 50 wt. %, less than 40 wt. %, less than 30 wt. %, or less than 20 wt. % primary polymer. In some embodiments, the second polymer composition may include more than 20 wt. %, more than 30 wt. %, more than 40 wt. %, more than 50 wt. %, more than 60 wt. %, more than 70 wt. %, or more than 80 wt. % primary polymer.

In some embodiments, the second polymer composition may additionally include one or more water-soluble excipients (which may include one or more polymers to the primary polymer described previously). For example, suitable water-soluble excipients may include a copovidone, a poloxamer, and/or a polyethylene oxide. Suitable commercially-available water-soluble excipients can include Kolliphor P407 (poloxamer 407, poly(ethylene glycol)-block-poly(propylene glycol)-block-poly(ethylene glycol)), PEG-PCL, SIF (FaSSIF/FaSSGF powder from BioRelevant), EPO (dimethylaminoethyl methacrylate-butyl methacrylate-methyl methacrylate copolymer), Kollidon VA64 (vinylpyrrolidone-vinyl acetate copolymer in a ratio of 6:4 by mass), polyvinyl pyrrolidine.

The second polymer composition may comprise 5-70 wt. % or 10-40 wt. % water-soluble excipients. In some embodiments, the second polymer composition may comprise less than 70 wt. %, less than 60 wt. %, less than 50 wt. %, less than 40 wt. %, less than 30 wt. %, less than 20 wt. %, or less than 10 wt. % water-soluble excipients. In some embodiments, the second polymer composition may comprise more than 5 wt. %, more than 10 wt. %, more than 20 wt. %, more than 30 wt. %, more than 40 wt. %, more than 50 wt. %, or more than 60 wt. % water-soluble excipients.

In some embodiments, the second polymer composition may comprise additional excipients. For example, the second polymer composition may comprise bismuth subcarbonate, silica, vitamin E succinate, iron oxide, a polyethylene glycol, polyvinyl acetate and polyvinylcaprolactame-based graft copolymer (Soluplus®), sodium starch glycolate, and/or hydroxypropyl cellulose. In some embodiments, the second polymer composition may comprise 10-70 wt. % or 20-50 wt. % excipients. In some embodiments, the second polymer composition may comprise less than 70 wt. %, less than 60 wt. %, less than 50 wt. %, less than 40 wt. %, less than 30 wt. %, or less than 20 wt. % excipients. In some embodiments, the second polymer composition may comprise more than 10 wt. %, more than 20 wt. %, more than 30 wt. %, more than 40 wt. %, more than 50 wt. %, or more than 60 wt. % excipients.

In some embodiments, the second polymer composition may additionally comprise an agent or API. The second polymer composition may comprise 20-80 wt. % or 40-60 wt. % agent or API. In some embodiments, the second polymer composition may comprise less than 80 wt. %, less than 70 wt. %, less than 60 wt. %, less than 50 wt. %, less than 40 wt. %, or less than 30 wt. % agent or API. In some embodiments, the second polymer composition may comprise more than 20 wt. %, more than 30 wt. %, more than 40 wt. %, more than 50 wt. %, more than 60 wt. %, or more than 70 wt. % agent or API.

Some polymer materials that are useful for creating arms of controlled stiffness may have an added advantage in thermal stability. For example, gastric residence systems may experience temperature variation during shipping and distribution. Shipping data suggest that cargo temperature extremes may approach 60° C. in some climates (Singh et al, Packag. Technol. Sci. 2012; 25: 149-160). The polymers that comprise gastric residence systems should be physically stable at this temperature if they are to be shipped without cold chain packaging and storage.

Polycaprolactone is a preferred polymer for relatively stiff arms (or stiff/first segments), and thermoplastic polyurethane is a preferred polymer for creating arms of controlled stiffness (i.e., second segments). Polycaprolactone-based arms are physically stable when exposed to temperatures as high as 55° C., but melt if they reach 60° C. When stored in a capsule, arms that begin to melt can adhere to one another and prevent the gastric residence system from unfolding in the stomach. Thermoplastic polyurethanes such as Pathway PY-PT72AE provide improved thermal stability. Pathway PY-PT72AE is an amorphous material that does not undergo a clear melt transition but does soften at elevated temperatures.

When a gastric residence system comprising relatively stiff arms is compressed (e.g., by gastric waves or a radial compression test), the compression force is transferred to the more flexible core of the gastric residence system, resulting in a gastric residence system in a bended configuration that is capable of passing through the pylorus of a patient (i.e., an opening having a diameter of 20 mm).

Conversely, when a gastric residence system comprising relatively flexible arms (i.e., having a first segment and a second segment) is compressed, the second segment absorbs some of the compression force. Thus, the compression forces are not transferred to the core of the gastric residence system having relatively flexible arms as is the case with the gastric residence system having relatively stiff arms. To compress the stiff inner segments of the arms to pyloric size, greater force is required due to the shorter lever arm attached to the flexible core. This can mean that the gastric residence system having relatively flexible arms requires a greater compression force to bend it into a configuration small enough to pass through the pylorus of a patient (i.e., an opening having a diameter of 20 mm).

As the second segment of an arm of a gastric residence system having arms of controlled stiffness increases relative to the first segment, so too does the compression force required to compress the gastric residence system into a bended configuration small enough to pass through a pylorus (i.e., an opening having a diameter of 20 mm). (As long as the size of the stiff inner portion and the core is still larger than the diameter of the pylorus.)

The ratio of the first segment of a relatively flexible arm to the second segment of the arm may vary. If the first segment is too large in comparison to the second segment, the compression forces may transfer to the core of a gastric residence system too early, allowing the compression forces to compress the gastric residence system into a bended configuration small enough to prematurely pass through a pylorus. If the second segment is too large compared to the first segment, the second segment may too easily bend under the compression forces, allowing the forces to compress the gastric residence system into a bended configuration small enough to prematurely pass through a pylorus. Both scenarios result in a gastric residence system that is not as effective at resisting premature passage through the pylorus as desired.

An effective ratio of the first segment to the second segment of a flexible arm of a gastric residence system may vary. In some embodiments, the first segment may comprise from 10-90% of a length of an arm (as measured from the proximal end to the distal end). In some embodiments, the first segment may comprise less than 90%, less than 80%, less than 70%, less than 60%, less than 50%, less than 40%, less than 30%, or less than 20% of a length of an arm. In some embodiments, the first segment may comprise more than 10%, more than 20%, more than 30%, more than 40%, more than 50%, more than 60%, more than 70%, or more than 80% of a length of an arm. In some embodiments, the second segment may comprise from 10-90% of a length of an arm (as measured from the proximal end to the distal end). In some embodiments, the second segment may comprise less than 90%, less than 80%, less than 70%, less than 60%, less than 50%, less than 40%, less than 30%, or less than 20% of a length of an arm. In some embodiments, the second segment may comprise more than 10%, more than 20%, more than 30%, more than 40%, more than 50%, more than 60%, more than 70%, or more than 80% of a length of an arm.

### System Polymeric Composition

The choice of the individual polymers for the carrier polymer, coupling polymer, and elastomer influence many properties of the system, such as drug elution rate (dependent on the carrier polymer, as well as other factors), the residence time of the system (dependent on the degradation of any of the polymers, principally the coupling polymers), the uncoupling time of the system if it passes into the intestine (dependent primarily on the enteric degradation rate of the coupling polymer, as discussed herein), and the shelf life of the system in its compressed form (dependent primarily on properties of the elastomer). As the systems will be administered to the gastrointestinal tract, all of the system components should be biocompatible with the gastrointestinal environment.

The rate of elution of drug from the carrier polymer-drug component is affected by numerous factors, including the composition and properties of the carrier polymer, which may itself be a mixture of several polymeric and non-polymeric components; the properties of the drug such as hydrophilicity/hydrophobicity, charge state, pKa, and hydrogen bonding capacity; and the properties of the gastric environment. In the aqueous environment of the stomach, avoiding burst release of a drug (where burst release refers to a high initial delivery of active pharmaceutical ingredient upon initial deployment of the system in the stomach), particularly a hydrophilic drug, and maintaining sustained release of the drug over a period of time of days to one or two weeks is challenging.

The residence time of the systems in the stomach is adjusted by the choice of coupling polymers used in the linker regions. The systems will eventually break down in the stomach, despite the use of enteric coupling polymers, as the mechanical action of the stomach and fluctuating pH will eventually weaken the enteric coupling polymers. Coupling polymers which degrade in a time-dependent manner in the stomach can also be used to adjust the time until the system breaks apart or weakens to the point where it can no longer resist passage through the pylorus, and hence adjust the residence time. Once the system breaks apart or weakens to the point where it can no longer resist passage through the pylorus, it passes into the intestines and is then eliminated.

The elastomer used in the systems is central to the shelf life of the systems. When the systems are compressed, the elastomer is subjected to mechanical stress. The stress in turn can cause polymer creep, which, if extensive enough, can prevent the systems from returning to their uncompacted configurations when released from the capsules or other container; this in turn would lead to premature passage of the system from the stomach. Polymer creep can also be temperature dependent, and therefore the expected storage conditions of the systems also need to be considered when choosing the elastomer and other polymer components.

The system components and polymers should not swell, or should have minimal swelling, in the gastric environment. The components should swell no more than about 20%, no more than about 10%, or no more than about 5% when in the gastric environment over the period of residence.

The systems are optionally radiopaque, so that they can be located via abdominal X-ray if necessary. In some embodiments, one or more of the materials used for construction of the system is sufficiently radiopaque for X-ray visualization. In other embodiments, a radiopaque substance is added to one or more materials of the system, or coated onto one or more materials of the system, or are added to a small portion of the system. Examples of suitable radiopaque substances are barium sulfate, bismuth subcarbonate, bismuth oxychloride, and bismuth trioxide. These materials are typically blended into a separate piece of the gastric residence system, such as a small segment in one or more of the arms, so as not to alter drug release from the carrier polymer, or desired properties of other system polymers such as the linkers or central elastomer. Metal striping or tips on a small portion of the system components can also be used, such as tungsten.

### Methods of Manufacture: Manufacture of Gastric Residence Systems

The gastric residence system or components thereof can be produced using three-dimensional printing techniques. Three-dimensional printing of components of the gastric residence system, such as arm or arm segments, is performed using commercially-available equipment. Three-dimensional printing has been used for pharmaceutical preparation; see Khaled et al., “Desktop 3D printing of controlled release pharmaceutical bilayer tablets,” International Journal of Pharmaceutics 461:105-111 (2014); U.S. Pat. No. 7,276,252; Alhnan et al., “Emergence of 3D Printed Dosage Forms: Opportunities and Challenges,” Pharm. Res., May 18, 2016, PubMed PMID: 27194002); Yu et al., “Three-dimensional printing in pharmaceutics: promises and problems,” J. Pharm. Sci. 97(9):3666-3690 (2008); and Ursan et al., “Three-dimensional drug printing: A structured review,” J. Am. Pharm. Assoc. 53(2):136-44 (2013).

The initial feedstocks for three-dimensional printing are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the segment or arm to be manufactured are mixed and pelletized using hot melt extrusion. The polymer or blended polymer material is extruded through a circular die, creating a cylindrical fiber which is wound around a spool.

Multiple spools are fed into the 3D printer (such as a Hyrel Printer, available from Hyrel 3D, Norcross, Ga., United States), to be fed into their representative print heads. The print heads heat up and melt the material at the nozzle, and lay down a thin layer of material (polymer or polymer blend) in a specific position on the piece being manufactured. The material cools and hardens within seconds, and the next layer is added until the complete structure is formed. The quality of the dosage form is dependent on the feed rate, nozzle temperature, and printer resolution; feed rate and nozzle temperature can be adjusted to obtain the desired quality.

Three-dimensional printing can be used to manufacture individual arms, or segments of arms. Three-dimensional printing can also be used to prepare a bulk configuration, such as a consolidated “slab,” similar to that prepared by co-extrusion methods described herein. The bulk configuration can be cut into individual pieces (that is, individual arms or individual segments) as needed.

In some embodiments of the invention, producing an entire arm of the gastric residence system by three-dimensional printing of the arm is contemplated. In some embodiments of the invention, producing a segment of an arm of the gastric residence system by three-dimensional printing of the segment of an arm is contemplated. In some embodiments, an arm or a segment thereof is produced by three-dimensional printing of adjacent portions of carrier polymer-agent or polymer-agent salt blend and linker material in a bulk configuration, such as a slab configuration. The three-dimensional printing can be followed by cutting the bulk configuration into pieces which have the desired shape of the arm or segment thereof. The three-dimensional printing can be followed by compression molding of portions of the bulk configuration into pieces which have the desired shape of the arm or segment thereof.

Three-dimensional printing is often accomplished by feeding a rod or fiber of a solid material to a print head, where it is melted and deposited with subsequent solidification, in a technique known as fused deposition modeling (sometimes also called extrusion deposition); see U.S. Pat. Nos. 5,121,329 and 5,340,433. The methods described herein for the manufacture of carrier polymer-drug components can also be used to manufacture feed material, which can be used in the manufacture via three-dimensional printing of components of the gastric residence systems.

Components of the gastric residence systems can alternatively be manufactured by co-extrusion. Most of the various configurations for the segments discussed herein can be made by either three-dimensional printing or co-extrusion. However, co-extrusion is less expensive, and can be run as a continuous process, as opposed to three-dimensional printing, which is generally run as a batch process.

Co-extrusion of components of the gastric residence system, such as an arm, or a segment of an arm, can be performed using commercially-available equipment, combined with customized co-extruder plumbing and customized dies for the desired configuration. The initial feedstocks for co-extrusion are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the segment or arm to be manufactured are mixed and pelletized using hot melt extrusion. The polymer pellets thus formed are placed into hoppers above single screw extruders and dried to remove surface moisture. Pellets are gravimetrically fed into individual single-screw extruders, where they are melted and pressurized for co-extrusion.

The appropriate molten polymers are then pumped through custom designed dies with multiple channels where they form the required geometry. The composite polymer block is cooled (water-cooled, air-cooled, or both) and cut or stamped into the desired shape, including, but not limited to, such shapes as triangular prisms, rectangular prisms, or cylinder sections (pie-shaped wedges).

In some embodiments of the invention, producing an entire arm of the gastric residence system by co-extruding the arm is contemplated. In some embodiments of the invention, producing a segment of an arm of the gastric residence system by co-extruding the segment of an arm is contemplated. In some embodiments, an arm or a segment thereof is produced by co-extruding adjacent portions of carrier polymer-agent or carrier polymer-agent salt blend and linker material in a bulk configuration, such as a slab configuration. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

In some embodiments, an arm or a segment thereof is produced by co-extruding adjacent portions of carrier polymer-agent or carrier polymer-agent salt blend and linker material in a bulk configuration, such as a slab configuration, while also co-extruding an additional polymer or polymers within the carrier polymer-agent or carrier polymer-agent salt blend, the linker material, or both the carrier polymer-agent (or agent salt) blend and the linker material. The co-extruding the additional polymer or polymers within the carrier polymer-agent or carrier polymer-agent salt blend, the linker material, or both the carrier polymer-agent (or agent salt) blend and the linker material can be performed in an islands-in-the-sea configuration. The co-extruding can be followed by cutting the bulk configuration into pieces which have the desired shape of the arm or segment thereof. The co-extruding can be followed by compression molding of portions of the bulk configuration into pieces which have the desired shape of the arm or segment thereof.

### Attachment of the Flexible Line

As described, a flexible line may be connected to the distal ends of the arms of the gastric residence system. The flexible line may be attached to a separate tip portion of at least one of the arms. In some embodiments, the flexible line may be attached to each arm by laser welding. The core and arms of the gastric residence system may be pre-assembled as described above (e.g., by placing the core and arms in a mold and joining them together in the mold, injection molding, 3D printing, or any other suitable method) and placed in a welding puck. The welding puck may comprise grooves in the same shape as the body of the gastric residence system (e.g., a stellate shape with arms having triangular cross-sections), such that the gastric residence system can be placed in the grooves of the welding puck to keep the gastric residence system in place during laser welding. The welding puck may further comprise one or more flexible line slots at the distal end of each arm through which the flexible line can be placed. For example, FIGS. 7A-7D show welding pucks with various flexible line slot configurations. FIG. 7A illustrates a welding puck 700a comprising stellate-shaped grooves into which a gastric residence system can be placed. Each arm groove 702a comprises a flexible line slot 704a (only one of each is labeled for the sake of brevity). Flexible line slot 704a has a width of about 1.4 mm and a depth of about 1.8 mm. Thus, welding puck 700a can accommodate a flexible line with a diameter of up to about 1.4 mm. FIG. 7B illustrates another welding puck 700b with stellate-shaped grooves. Each arm groove 702b comprises a flexible line slot 704b which is deeper than the flexible line slot shown in FIG. 7A. Flexible line slot 704b has a width of about 1.4 mm and a depth of about 2.7 mm. FIG. 7C illustrates another welding puck 700c with stellate-shaped grooves. Each arm groove 702c comprises a flexible line slot 704c which is narrower than the flexible line slots shown in FIGS. 7A-7B. Flexible line slot 704c has a width of about 0.7 mm and a depth of about 1.8 mm. Thus, welding puck 700c can accommodate a flexible line with a diameter of up to about 0.7 mm. FIG. 7D illustrates another welding puck 700d with stellate-shaped grooves. Five of the arm grooves 702d include a single flexible line slot 704d. Each single flexible line slot 704d has a width of about 1.4 mm and a depth of about 1.8 mm. A sixth arm groove 702d includes a double flexible line slot 706. Double flexible line slot 706 allows a flexible line to be wrapped around the entire circumference of the gastric residence system with each end secured in a slot of double flexible line slot 706. Each slot of double flexible line slot 706 has a width of about 1.4 mm and a depth of about 1.8 mm.

In some embodiments, the welding puck may comprise stainless steel (such as stainless steel 316) and/or aluminum. In some embodiments, the welding puck may comprise a silicone skin. The silicone skin may mitigate flash effects during welding. Welding flash can cause capsule bulging when the gastric residence system is folded and placed into a capsule or prevent encapsulation altogether. The silicone skin may be produced by injection molding. In some embodiments, the silicone skin may comprise a silicone elastomer with a tensile strength of about 500-2000 psi and a shore A hardness of about 40-70 A (e.g., Dow Corning QP1-250). To further mitigate welding flash, the welding puck may include a top layer that may be placed on top of the welding puck during laser welding. The top layer may prevent the material that is melted during laser welding from flowing into the flexible line slots of the welding puck and causing welding flash. The top layer may be produced by injection molding or by die punching. In some embodiment, the top layer may comprise silicone. As a non-limiting example, the top layer may comprise SSP 2390-40 silicone (tensile strength of about 1310 psi (about 9 MPa), Shore A hardness about 39), which may be about 1/32 inches (0.8 mm) thick, or Dow Corning QP1-250.

Once the gastric residence system is placed in the welding puck, the flexible line may be embedded within the distal ends of the arms of the gastric residence system using laser welding. A laser may be directed at the distal end of each arm of the gastric residence system. The laser may at least partially melt each distal end, such that the distal end becomes soft enough to embed the flexible line within it. Once the distal end is softened, the flexible line may be placed in the welding puck and aligned with the flexible line slots. The flexible line may then be pressed down into the flexible line slots. The distal ends may then be allowed to cool and harden, such that the flexible line becomes embedded within the cooled and hardened distal ends. An example of a gastric residence system with an embedded flexible line is shown in FIG. 8.

Alternatively, or additionally, the flexible line may be embedded within the distal ends of the arms of the gastric residence system using techniques other than laser welding. For example, the flexible line may be embedded by heating the distal ends of the arms using convection heating, which may at least partially melt the distal ends. A portion of the flexible line may be embedded in each arm by pressing the flexible line into the distal ends and allowing the distal ends to cool with the flexible line embedded.

### Agent Particle Size and Milling

Control of particle size used in the gastric residence systems is important for both optimal release of agent and mechanical stability of the systems. The particle size of the agents affects the surface area of the agents available for dissolution when gastric fluid permeates the carrier polymer-agent segments of the system. Also, as the arms of the systems are relatively thin in diameter (for example, 1 millimeter to 5 millimeters), the presence of a particle of agent of a size in excess of a few percent of the diameter of the arms will result in a weaker arm, both before the agent elutes from the device, and after elution when a void is left in the space formerly occupied by the agent particle. Such weakening of the arms is disadvantageous, as it may lead to premature breakage and passage of the system before the end of the desired residence period.

In one embodiment, the agent particles used for blending into the carrier polymer-agent components are smaller than about 100 microns in diameter. In another embodiment, the agent particles are smaller than about 75 microns in diameter. In another embodiment, the agent particles are smaller than about 50 microns in diameter. In another embodiment, the agent particles are smaller than about 40 microns in diameter. In another embodiment, the agent particles are smaller than about 30 microns in diameter. In another embodiment, the agent particles are smaller than about 25 microns in diameter. In another embodiment, the agent particles are smaller than about 20 microns in diameter. In another embodiment, the agent particles are smaller than about 10 microns in diameter. In another embodiment, the agent particles are smaller than about 5 microns in diameter.

In one embodiment, at least about 80% of the agent particles used for blending into the carrier polymer-agent components are smaller than about 100 microns in diameter. In another embodiment, at least about 80% of the agent particles are smaller than about 75 microns in diameter. In another embodiment, at least about 80% of the agent particles are smaller than about 50 microns in diameter. In another embodiment, at least about 80% of the agent particles are smaller than about 40 microns in diameter. In another embodiment, at least about 80% of the agent particles are smaller than about 30 microns in diameter. In another embodiment, at least about 80% of the agent particles are smaller than about 25 microns in diameter. In another embodiment, at least about 80% of the agent particles are smaller than about 20 microns in diameter. In another embodiment, at least about 80% of the agent particles are smaller than about 10 microns in diameter. In another embodiment, at least about 80% of the agent particles are smaller than about 5 microns in diameter.

In one embodiment, at least about 80% of the mass of the agent particles used for blending into the carrier polymer-agent components have sizes between about 1 micron and about 100 microns in diameter. In another embodiment, at least about 80% of the mass of the agent particles have sizes between about 1 micron and about 75 microns in diameter. In another embodiment, at least about 80% of the mass of the agent particles have sizes between about 1 micron and about 50 microns in diameter. In another embodiment, at least about 80% of the mass of the agent particles have sizes between about 1 micron and about 40 microns in diameter. In another embodiment, at least about 80% of the mass of the agent particles have sizes between about 1 micron and about 30 microns in diameter. In another embodiment, at least about 80% of the mass of the agent particles have sizes between about 1 micron and about 25 microns in diameter. In another embodiment, at least about 80% of the mass of the agent particles have sizes between about 1 micron and about 20 microns in diameter. In another embodiment, at least about 80% of the mass of the agent particles have sizes between about 1 micron and about 10 microns in diameter. In another embodiment, at least about 80% of the mass of the agent particles have sizes between about 1 micron and about 5 microns in diameter.

In one embodiment, at least about 80% of the mass of the agent particles used for blending into the carrier polymer-agent components have sizes between about 2 microns and about 100 microns in diameter. In another embodiment, at least about 80% of the mass of the agent particles have sizes between about 2 microns and about 75 microns in diameter. In another embodiment, at least about 80% of the mass of the agent particles have sizes between about 2 microns and about 50 microns in diameter. In another embodiment, at least about 80% of the mass of the agent particles have sizes between about 2 microns and about 40 microns in diameter. In another embodiment, at least about 80% of the mass of the agent particles have sizes between about 2 microns and about 30 microns in diameter. In another embodiment, at least about 80% of the mass of the agent particles have sizes between about 2 microns and about 25 microns in diameter. In another embodiment, at least about 80% of the mass of the agent particles have sizes between about 2 microns and about 20 microns in diameter. In another embodiment, at least about 80% of the mass of the agent particles have sizes between about 2 microns and about 10 microns in diameter. In another embodiment, at least about 80% of the mass of the agent particles have sizes between about 2 microns and about 5 microns in diameter.

In one embodiment, at least about 80% of the mass of the agent particles used for blending into the carrier polymer-agent components have sizes between about 5 microns and about 100 microns in diameter. In another embodiment, at least about 80% of the mass of the agent particles have sizes between about 5 microns and about 75 microns in diameter. In another embodiment, at least about 80% of the mass of the agent particles have sizes between about 5 microns and about 50 microns in diameter. In another embodiment, at least about 80% of the mass of the agent particles have sizes between about 5 microns and about 40 microns in diameter. In another embodiment, at least about 80% of the mass of the agent particles have sizes between about 5 microns and about 30 microns in diameter. In another embodiment, at least about 80% of the mass of the agent particles have sizes between about 5 microns and about 25 microns in diameter. In another embodiment, at least about 80% of the mass of the agent particles have sizes between about 5 microns and about 20 microns in diameter. In another embodiment, at least about 80% of the mass of the agent particles have sizes between about 5 microns and about 10 microns in diameter.

The particle size of the agents can be readily adjusted by milling. Several milling techniques are available to reduce larger particles to smaller particles of desired size. Fluid energy milling is a dry milling technique which uses inter-particle collisions to reduce the size of particles. A type of fluid energy mill called an air jet mill shoots air into a cylindrical chamber in a manner so as to maximize collision between agent particles. Ball milling utilizes a rolling cylindrical chamber which rotates around its principal axis. The agent and grinding material (such as steel balls, made from chrome steel or CR-NI steel; ceramic balls, such as zirconia; or plastic polyamides) collide, causing reduction in particle size of the agent. Ball milling can be performed in either the dry state, or with liquid added to the cylinder where the agent and the grinding material are insoluble in the liquid. Further information regarding milling is described in the chapter by R. W. Lee et al. entitled “Particle Size Reduction” in Water-Insoluble Drug Formulation, Second Edition (Ron Liu, editor), Boca Raton, Fla.: CRC Press, 2008; and in the chapter by A. W. Brzeczko et al. entitled “Granulation of Poorly Water-Soluble Drugs” in Handbook of Pharmaceutical Granulation Technology, Third Edition (Dilip M. Parikh, editor), Boca Raton, Fla.: CRC Press/Taylor & Francis Group, 2010 (and other sections of that handbook). Fluid energy milling (i.e., air jet milling) is a useful method of milling, as it is more amenable to scale-up compared to other dry milling techniques such as ball milling.

Substances can be added to the agent material during milling to assist in obtaining particles of the desired size, and minimize aggregation during handling. Silica (silicon dioxide, SiO2) is a useful milling additive, as it is inexpensive, widely available, and non-toxic. Other additives which can be used include silica, calcium phosphate, powdered cellulose, colloidal silicon dioxide, hydrophobic colloidal silica, magnesium oxide, magnesium silicate, magnesium trisilicate, talc, polyvinylpyrrolidone, cellulose ethers, polyethylene glycol, polyvinyl alcohol, and surfactants. In particular, hydrophobic particles less than 5 microns in diameter are particularly prone to agglomeration, and hydrophilic additives are used when milling such particles. A weight/weight ratio of about 0.1% to about 5% of milling additive, such as silica, can be used for fluid milling or ball milling, or about 0.1% to about 4%, about 0.1% to about 3%, about 0.1% to about 2%, about 0.1% to about 1%, about 1% to about 5%, about 1% to about 4%, about 1% to about 3%, about 1% to about 2%, or about 0.1%, about 0.5%, about 1%, about 2%, about 3%, about 4% or about 5%.

After milling, particles can be passed through meshes of appropriate size to obtain particles of the desired size. To obtain particles of a desired maximum size, particles are passed through a mesh with holes of the maximum size desired; particles which are too large will be retained on the mesh, and particles which pass through the mesh will have the desired maximum size. To obtain particles of a desired minimum size, particles are passed through a mesh with holes of the minimum size desired; particles which pass through the mesh are too small, and the desired particles will be retained on the mesh.

### Methods of Manufacture of Carrier Polymer-Agent (or Agent Salt) Components

Blending temperatures for incorporation of the agent (or a pharmaceutically acceptable salt thereof) into polymeric matrices typically range from about 80° C. to about 120° C., although higher or lower temperatures can be used for polymers which are best blended at temperatures outside that range. When agent (or salt thereof) particles of a particular size are used, and it is desired that the size of the particles be maintained during and after blending, blending can be done at temperatures below the melting point of the agent (or salt thereof), so as to maintain the desired size of the particles. Otherwise, temperatures can be used which melt both the polymer and the agent (or salt thereof). Blending temperatures should be below the degradation temperature of the agent (or salt thereof). In one embodiment, less than about 2% of the agent (or salt thereof) is degraded during manufacture. In one embodiment, less than about 1.5% of the agent (or salt thereof) is degraded during manufacture. In one embodiment, less than about 1% of the agent (or salt thereof) is degraded during manufacture. In one embodiment, less than about 0.75% of the agent (or salt thereof) is degraded during manufacture. In one embodiment, less than about 0.5% of the agent (or salt thereof) is degraded during manufacture. In one embodiment, less than about 0.4% of the agent (or salt thereof) is degraded during manufacture. In one embodiment, less than about 0.3% of the agent (or salt thereof) is degraded during manufacture. In one embodiment, less than about 0.2% of the agent (or salt thereof) is degraded during manufacture. In one embodiment, less than about 0.15% of the agent (or salt thereof) is degraded during manufacture. In one embodiment, less than about 0.1% of the agent (or salt thereof) is degraded during manufacture. In one embodiment, less than about 0.05% of the agent (or salt thereof) is degraded during manufacture. In one embodiment, less than about 0.04% of the agent (or salt thereof) is degraded during manufacture. In one embodiment, less than about 0.03% of the agent (or salt thereof) is degraded during manufacture. In one embodiment, less than about 0.02% of the agent (or salt thereof) is degraded during manufacture. In one embodiment, less than about 0.01% of the agent (or salt thereof) is degraded during manufacture.

Hot melt extrusion can be used to prepare the carrier polymer-agent (or agent salt) components. Single-screw or twin-screw systems can be used. As noted, if it is desired that the size of the particles be maintained during and after blending, carrier polymers should be used which can be melted at temperatures which do not degrade the agent or salt thereof. Otherwise, temperatures can be used which melt both the polymer and the agent or salt thereof.

Melting and casting can also be used to prepare the carrier polymer-agent (or salt thereof) components. The carrier polymer and agent (or salt thereof), and any other desired components, are mixed together. The carrier polymer is melted and the melt is mixed so that the agent (or salt thereof) particles are evenly distributed in the melt, poured into a mold, and allowed to cool.

Solvent casting can also be used to prepare the carrier polymer-agent (or salt thereof) components. The polymer is dissolved in a solvent, and particles of agent (or salt thereof) are added. If the size of the agent (or salt thereof) particles are to be maintained, a solvent should be used which does not dissolve the agent (or salt thereof) particles, so as to avoid altering the size characteristics of the particles; otherwise, a solvent which dissolves both the polymer and agent (or salt thereof) particles can be used. The solvent-carrier polymer-agent (or salt thereof) particle mixture (or solvent-carrier particle-agent/agent salt solution), is then mixed to evenly distribute the particles (or thoroughly mix the solution), poured into a mold, and the solvent is evaporated.

### Manufacture/Assembly of System: Affixing Arms to Central Elastomer

For a stellate gastric residence system, such as that shown in FIG. 1A, the arms of the gastric residence system can be affixed to the central elastomer in a number of ways. The central polymer can be cast or molded with short “asterisk” arms, and a linker polymer can be used to affix the arms to the asterisk arms of the central elastomer. Alternatively, the central elastomer can be formed in a mold into which the proximal ends of the arms protrude. The elastomer sets, cures, or otherwise hardens into its desired form with a portion of the arms extending into the body of the central elastomer. Alternatively, the central elastomer can be prepared with cavities into which the arms can be firmly inserted.

The invention thus includes a method of making a gastric residence system, comprising preparing at least three arms formed from a material comprising any drug-carrier polymer-excipient formulation as disclosed herein; and attaching the arms to a central elastomer to form a gastric residence system. The arms can comprise at least one segment with a release rate-controlling polymer film. The arms of the gastric residence system project radially from the central elastomer, such as in a “hub and spoke” arrangement. A preferred number of arms is six. However, stellate systems with three, four, five, seven, or eight arms can also be used.

In some embodiments, arms comprising any carrier polymer-agent formulation can be heat-welded, solvent-welded, or otherwise affixed to other elements, including disintegrating matrices, coupling polymers, or interfacing polymers, which are then affixed to a central elastomer. In some embodiments, the arms are directly affixed to a central elastomer. Disintegrating matrices, coupling polymers, or interfacing polymer segments can be welded or otherwise affixed to the central elastomer prior to affixing the arms.

In some embodiments, arms comprising any drug-carrier polymer-excipient formulation as disclosed herein can be heat-welded to polycaprolactone segments, such as short polycaprolactone “asterisk” arms affixed to a central elastomer. Linker segments can be welded to the short “asterisk” arms prior to affixing the drug-carrier polymer-excipient formulation arms. Heat welding of drug-carrier polymer-excipient formulation arms to MW 80,000 PCL segments at temperatures between 140° C. to 170° C., followed by cooling for 24 hours at 8° C., resulted in stronger welds. Thus, in one embodiment, attaching the arms comprising any drug-carrier polymer-excipient formulation as disclosed herein to a central elastomer to form a gastric residence system, can comprise heat-welding the arms to other system components, such as asterisk arms or other segments comprising at least about 90%, at least about 95%, or at least about 99% polycaprolactone (such as MW 80,000 PCL), at a temperature between about 140° C. to about 170° C., followed by cooling of the welded members attached to other system components for about 12 to about 48 hours at a temperature of about 2° C. to about 14° C., such as about 5° C. to about 10° C., or about 8° C. The other system components can alternatively be linker elements.

### Manufacture/Assembly of System

In some embodiments, once the arms of the gastric residence system have been affixed to the central elastomer, the system is ready to be folded into its compacted configuration and placed into a capsule for storage, transport, and eventual administration. The system can be folded in an automated mechanical process, or by hand, and placed into a capsule of the appropriate size and material. More detail regarding manufacture and assembly of gastric residence systems, and of packaging the gastric residence system into capsules, can be found in International Patent Application Nos. WO 2015/191920, WO 2015/191925, WO 2017/070612, WO 2017/100367, WO 2017/205844, and WO 2018/227147.

### Methods of Treatment Using the Gastric Residence Systems

The gastric residence systems can be used to treat conditions requiring administration of a drug or agent over an extended period of time. In one embodiment, a gastric residence system is administered to a human. For long-term administration of agents or drugs which are taken for months, years, or indefinitely, administration of a gastric residence system periodically, such as once weekly or once every two weeks can provide substantial advantages in patient compliance and convenience. Accordingly, the gastric residence systems of the invention can be administered once every three days, once every five days, once weekly, once every ten days, or once every two weeks. The administration frequency is timed to coincide with the designed gastric residence period of the gastric residence system which is administered, so that at about the same time that a gastric residence system passes out of the stomach after its residence period, a new gastric residence system is administered.

Once a gastric residence system has been administered to a patient, the system provides sustained release of agent or drug over the period of gastric retention. After the period of gastric retention, the system degrades and passes out of the stomach. Thus, for a system with a gastric retention period of one week, the patient will swallow (or have administered to the stomach via other methods) a new system every week. Accordingly, in one embodiment, a method of treatment of a patient with a gastric retention system of the invention having a gastric residence period of a number of days D (where D-days is the gastric residence period in days), over a total desired treatment period T-total (where T-total is the desired length of treatment in days) with the agent or drug in the system, comprises introducing a new gastric residence system every D-days into the stomach of the patient, by oral administration or other methods, over the total desired treatment period. The number of gastric residence systems administered to the patient will be (T-total) divided by (D-days). For example, if treatment of a patient for a year (T-total=365 days) is desired, and the gastric residence period of the system is 7 days (D-days=7 days), approximately 52 gastric residence systems will be administered to the patient over the 365 days, as a new system will be administered once every seven days.

Alternatively, the patient can swallow (or have administered to the stomach via other methods) a new gastric residence system at the end of the effective release period of the gastric residence system. The “effective release period” or “effective release time” is the time over which the gastric residence system releases an effective amount of the agent contained in the system. Accordingly, in one embodiment, a method of treatment of a patient with a gastric residence system of the invention having an effective release period of a number of days E (where E-days is the effective release period in days), over a total desired treatment period T-total (where T-total is the desired length of treatment in days) with the agent in the system, comprises introducing a new gastric residence system every E-days into the stomach of the patient, by oral administration or other means, over the total desired treatment period. The number of gastric residence systems administered to the patient will be (T-total) divided by (E-days). For example, if treatment of a patient for a year (T-total=365 days) is desired, and the effective release period of the system is 7 days (E-days=7 days), approximately 52 gastric residence systems will be administered to the patient over the 365 days, as a new system will be administered once every seven days.

### Kits and Articles of Manufacture

Also provided herein are kits for treatment of patients with the gastric residence systems of the invention. The kit may contain, for example, a sufficient number of gastric residence systems for periodic administration to a patient over a desired total treatment time period. If the total treatment time in days is (T-total), and the gastric residence systems have a residence time of (D-days), then the kit will contain a number of gastric residence systems equal to ((T-total) divided by (D-days)) (rounded to an integral number), for administration every D-days. Alternatively, if the total treatment time in days is (T-total), and the gastric residence systems have an effective release period of (E-days), then the kit will contain a number of gastric residence systems equal to ((T-total) divided by (E-days)) (rounded to an integral number), for administration every E-days. The kit may contain, for example, several gastric residence systems in containers (where the containers may be capsules) and may optionally also contain printed or computer readable instructions for dosing regimens, duration of treatment, or other information pertinent to the use of the gastric residence systems and/or the agent or drug contained in the gastric residence systems. For example, if the total treatment period prescribed for the patient is one year, and the gastric residence system has a residence time of one week or an effective release period of one week, the kit may contain 52 capsules, each capsule containing one gastric residence system, with instructions to swallow one capsule once a week on the same day (e.g., every Saturday).

Articles of manufacture, comprising a sufficient number of gastric residence systems for periodic administration to a patient over a desired total treatment time period, and optionally comprising instructions for dosing regimens, duration of treatment, or other information pertinent to the use of the gastric residence systems and/or the agent or drug contained in the gastric residence systems, are also included in the invention. The articles of manufacture may be supplied in appropriate packaging, such as dispensers, trays, or other packaging that assists the patient in administration of the gastric residence systems at the prescribed interval.

### Testing Methods

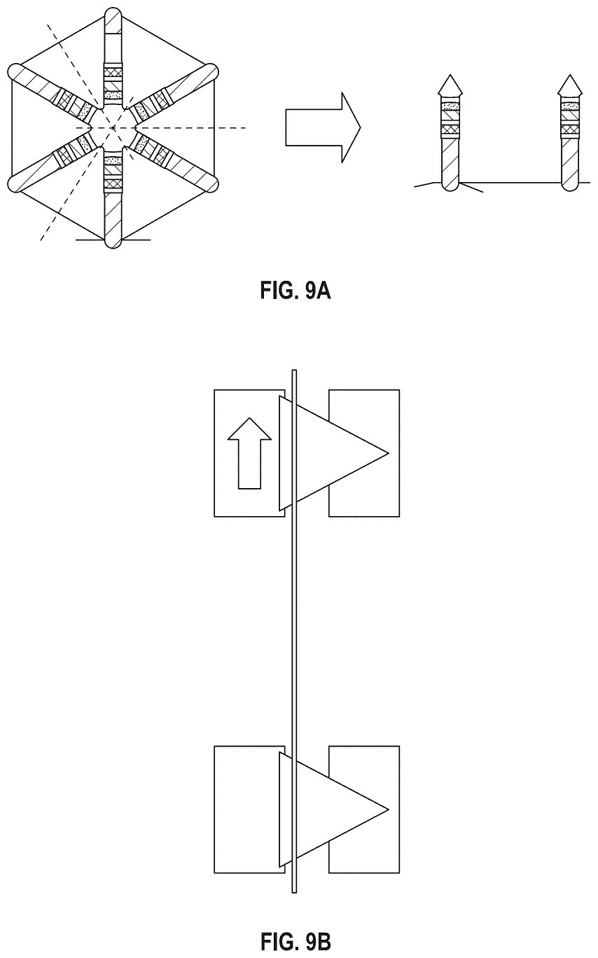

Flexible line adhesion, which is the amount of force required to separate a flexible line from a distal end of an arm, can be quantified using the Two Arm Pull (TAPS) test or the pullout force test. FIGS. 9A-9B illustrate the TAPS method. As shown in FIG. 9A, a gastric residence system having six arms and a flexible line may be prepared. The arms may be isolated from each other at a proximal end by cutting the elastomeric core into six parts. The flexible line may be cut into three parts to create three sets of two arms connected at a distal end by a portion of the flexible line. The dotted lines in FIG. 9A illustrate the cuts.

Using a set of two arms connected by a portion of the flexible line, the amount of force required to separate the flexible line from a distal end of an arm may then be tested. As shown in FIG. 9B, one arm may be placed in a top clamp and one arm may be placed in a bottom clamp with slack in the flexible line. Once the arms are secured, the top clamp may be moved upward to reduce (and eventually eliminate) the slack in the flexible line. The top clamp may be moved upward until the flexible line slips out of one or both of the arms. Thus, because the flexible line can be separated from either arm during the test, the TAPS method tests the minimum flexible line adhesion of the pair of arms rather than the flexible line adhesion of a flexible line to a single arm. The TAPS method is therefore a good prognosticator of the risk of flexible line slippage. The TAPS method may also measure the force required to break a flexible line. For example, moving the first clamp away from the second clamp may cause the flexible line to break instead of or in addition to slipping out of one or both of the arms, and the force required to break the flexible line may be measured.

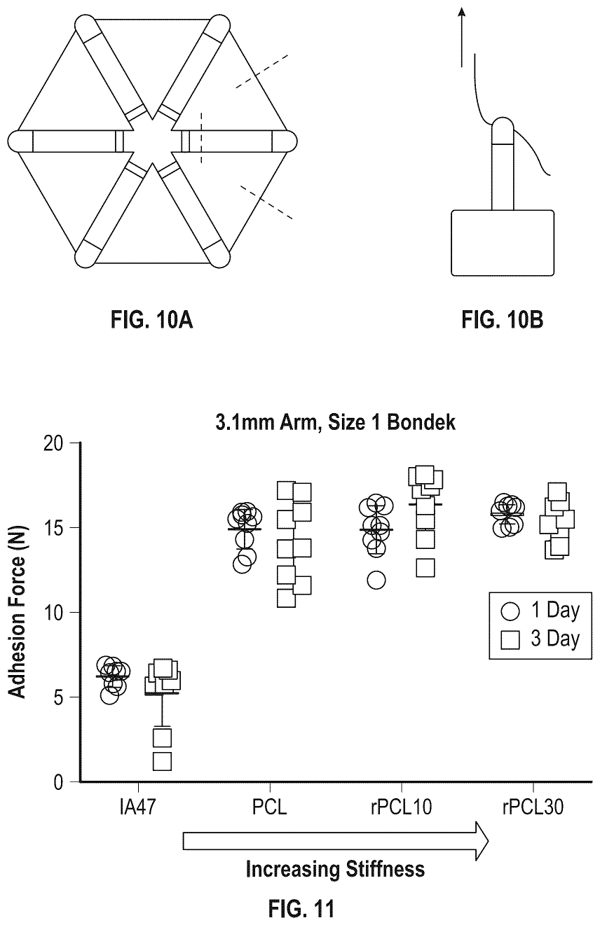

An alternative method for measuring flexible line adhesion is the pull force test, which is illustrated in FIGS. 10A-10B. As shown in FIG. 10A, the arms of a gastric residence system having six arms and flexible line a may be isolated by cutting the elastomeric core into six parts. The flexible line may be cut between each arm. The tensile force required to pull the flexible line out of each arm tip may be measured using an Instron 3340 Series Universal Testing System by gripping the base of the arm and one end of the flexible line.

### EXAMPLES

Example 1: The adhesion of a flexible line to various materials which can comprise the distal ends and/or separate tip portions of the arms of a gastric residence system was tested using the TAPS method described above in detail with reference to FIGS. 9A-9B. Gastric residence systems were prepared using a Size 1 Bondek® flexible line (comprising PGA). Four different materials having a cross-section thickness of 3.1 mm were tested: IA47 (66.45% Corbion® PC17, i.e., PCL with inherent viscosity midpoint of 1.7 dl/g, 32% VA64, 1.5% Poloxamer 407, 0.05% FD&C Blue 1 Aluminum Lake), PCL, rPCL10 (90% PCL, 10% bismuth), and rPCL30 (70% PCL, 30% bismuth). Each substrate tested had a cross-section thickness of 3.1 mm. Flexible line adhesion was measured after incubation in fasted state simulated gastric fluid (obtained from Biorelevant Ltd., London, United Kingdom; aqueous solution of 0.08 mM taurocholate, 0.02 mM phospholipids, 34 mM sodium ion, 59 mM chloride, pH 1.6) at 37° C. for 1 day or 3 days. As shown in FIG. 11, flexible line adhesion was stronger for stiffer materials, as measured by maximum force, flexural strength, and flexural modulus. The length of incubation (i.e., 1 day or 3 days) only marginally affected the flexible line adhesion of the samples tested.

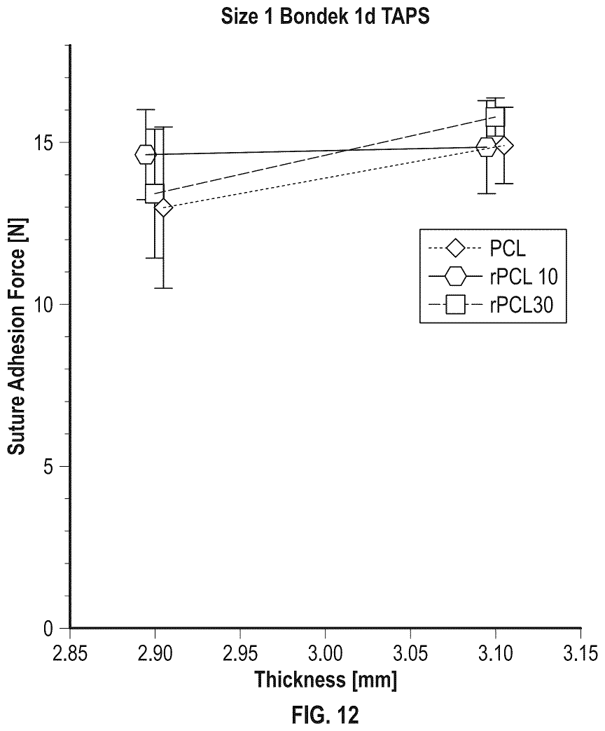

Example 2: The adhesion of a flexible line to distal end portions having varying cross-section thicknesses was tested using the TAPS method described above in detail with reference to FIGS. 9A-9B. Gastric residence systems were prepared using a Size 1 Bondek® flexible line (comprising PGA). Distal end portions were prepared using PCL, rPCL10 (10% bismuth), and rPCL30 (30% bismuth) at cross-section thicknesses of 2.90 mm and 3.10 mm. (Cross-section thickness, as used in this example, refers to the length of the base of the equilateral triangular cross-section of each arm.) Flexible line adhesion was measured after incubation in fasted state simulated gastric fluid at 37° C. for 1 day. As shown in FIG. 12, distal end portion thickness had only a marginal effect on adhesion force.

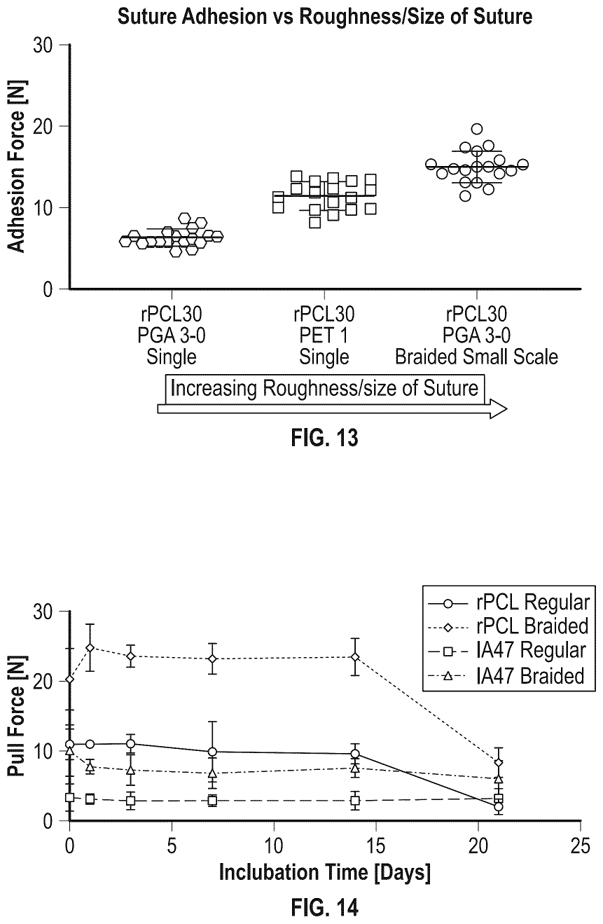

Example 3: The adhesion of a flexible line to distal end portions having varying surface areas and surface roughness was tested using the TAPS method described above in detail with reference to FIGS. 9A-9B. Gastric residence systems were prepared using flexible lines having varying sizes and compositions. A table correlating flexible line sizes to diameter ranges is provided below in Table 1. In the gastric residence systems tested, the distal end portions comprised rPCL30. As shown in FIG. 13, as roughness and surface area of a flexible line increased, flexible line adhesion also increased. For example, braided flexible line configurations experienced stronger flexible line adhesion than single flexible line configurations.

TABLE 1

| | Collagen Flexible Lines | | Synthetic Flexible Lines | |
|---|---|---|---|---|
| U.S.P. | Metric | Diameter | Metric | Diameter |
| Size | Size | Range | Size | Range |
| # 4 | 8 | 0.800-0.899 | 6 | 0.600-0.699 |
| # 3 | 7 | 0.700-0.799 | 6 | 0.600-0.699 |
| # 2 | 6 | 0.600-0.699 | 5 | 0.500-0.599 |
| # 1 | 5 | 0.500-0.599 | 4 | 0.400-0.499 |
| # 0 | 4 | 0.400-0.499 | 3.5 | 0.350-0.399 |
| # 2-0 | 3.5 | 0.350-0.399 | 3 | 0.300-0.349 |
| # 3-0 | 3 | 0.300-0.349 | 2 | 0.200-0.249 |
| # 4-0 | 2 | 0.200-0.249 | 1.5 | 0.150-0.199 |
| # 5-0 | 1.5 | 0.150-0.199 | 1 | 0.100-0.149 |
| # 6-0 | 1 | 0.100-0.149 | 0.7 | 0.070-0.099 |
| # 7-0 | 0.7 | 0.070-0.099 | 0.5 | 0.050-0.069 |
| # 8-0 | 0.5 | 0.050-0.069 | 0.4 | 0.040-0.049 |
| # 9-0 | 0.4 | 0.040-0.049 | 0.3 | 0.030-0.039 |
| # 10-0 | — | — | 0.2 | 0.020-0.029 |

Example 4: The adhesion of braided and single flexible lines to rPCL and IA47 distal end portions was tested using the pull test described above in detail with reference to FIGS. 10A-10B. The flexible lines were size 3-0 PGA flexible lines. For each combination of flexible line type and distal end portion composition, flexible line adhesion was measured after incubation in fasted state simulated gastric fluid at 37° C. for 1 day, 3 days, 7 days, 14 days, and 21 days. As shown in FIG. 14, braided flexible line configurations experienced higher flexible line adhesion to both rPCL and IA47 distal end portions, as compared to single-stranded flexible line configurations. Flexible line adhesion was fairly constant across all time points for IA47 distal end portions. Flexible line adhesion was also fairly constant over a 14-day time period for rPCL distal end portions, though a decrease in flexible line adhesion was observed over the 14-21 day time period. The combination of a braided flexible line and an rPCL distal end portion yielded the best flexible line adhesion of the combinations tested.

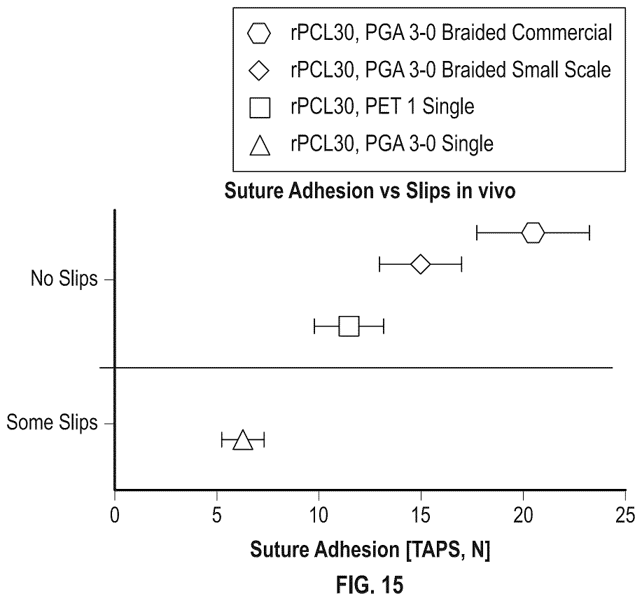

Example 5: The relationship between flexible line adhesion and flexible line slippage was tested using the TAPS method described above in detail with reference to FIGS. 9A-9B. Gastric residence systems were prepared using rPCL30 distal end portions and various flexible line materials and configurations. rPCL30 distal end portions were tested with size 1 single-stranded PET flexible lines, size 3-0 hand-braided PGA flexible lines, size 3-0 commercially braided PGA flexible lines, and size 3-0 single-stranded PGA flexible lines. As shown in FIG. 15, no flexible line slips were observed for braided flexible lines or size 1 PET flexible lines. Size 1 single-stranded PET flexible lines had a minimum flexible line adhesion of around 8 N, while size 3-0 hand-braided PGA flexible lines had a minimum flexible line adhesion of about 11 N and size 3-0 commercially braided PGA flexible lines had a minimum flexible line adhesion of about 14 N. Flexible line adhesion was strongest using braided flexible line configurations, specifically the size 3-0 commercially braided flexible lines. Flexible line adhesion was weakest for single-stranded flexible lines, and slips were observed for size 3-0 single-stranded PGA flexible lines.

Example 6: The safety and efficacy of a gastric residence system comprising risperidone was evaluated in a clinical study. A gastric residence system having a configuration described herein with reference to FIG. 16 was prepared. Specifically, the gastric residence system was a six-sided stellate having a core and six arms. One arm was a drug eluting arm comprising, in order from innermost segment closest to the core to outermost segment from the core, (1) a first inert segment, (2) a time-dependent disintegrating matrix segment, (3) a second inert segment, (4) an enteric disintegrating matrix segment, (5) a third inert segment, (6) an active pharmaceutical ingredient-containing portion, and (7) a fourth inert segment. The active pharmaceutical ingredient-containing portion comprised risperidone. The other arms were non-drug eluting arms comprising, in order from innermost segment closest to the core to outermost segment from the core, (1) a first inert segment, (2) a time-dependent disintegrating matrix segment, (3) a second inert segment, (4) an enteric disintegrating matrix segment, (5) a third inert segment, and (6) a fourth inert segment. The compositions and dimensions of each segment of the drug eluting arm and the non-drug eluting arms are provided in the table below.

| | Drug eluting arm | | Non-drug eluting arms | |
|---|---|---|---|---|
| | Composition | | Composition | |
| Segment | (% w/w) | Dimensions | (% w/w) | Dimensions |
| First inert | 100% PCL | 1.3 mm radial | 100% PCL | 1.3 mm radial |
| segment | | length/3.3 mm | | length/3.3 mm |
| | | triangular | | triangular |
| | | cross-section | | cross-section |
| | | base length | | base length |
| Time-dependent | 44.95% PCL12/2.0% | 1.0 mm radial | 44.95% PCL12/2.0% | 1.0 mm radial |
| disintegrating | PEO100K/53.0% 50/50 | length/3.3 mm | PEO100K/53.0% 50/50 | length/3.3 mm |
| matrix segment | DL-Lactide/Glycolide | triangular | DL-Lactide/Glycolide | triangular |
| | copolymer, acid | cross-section | copolymer, acid | cross-section |
| | (PDLG 5002A)/0.05% | base length | (PDLG 5002A)/0.05% | base length |
| | ferrosoferric oxide | | ferrosoferric oxide | |
| Second inert | 70% Corbion PC17/30% | 0.5 mm radial | 70% Corbion PC17/30% | 0.5 mm radial |
| segment | bismuth subcarbonate | length/3.3 mm | bismuth subcarbonate | length/3.3 mm |
| | | triangular | | triangular |
| | | cross-section | | cross-section |
| | | base length | | base length |
| Enteric | 41.5% PCL/56.5% | 1.85 mm radial | 41.5% PCL/56.5% | 1.85 mm radial |
| disintegrating | HPMCAS-MG/2.0% | length/3.3 mm | HPMCAS-MG/2.0% | length/3.3 mm |
| matrix segment | P407 | triangular | P407 | triangular |
| | | cross-section | | cross-section |
| | | base length | | base length |
| Third inert | 70% Corbion PC17/30% | 0.5 mm radial | 70% Corbion PC17/30% | 0.5 mm radial |
| segment | bismuth subcarbonate | length/3.3 mm | bismuth subcarbonate | length/3.3 mm |
| | | triangular | | triangular |
| | | cross-section | | cross-section |
| | | base length | | base length |
| Active | 35.0% Risperidone/55.9% | 7.3 mm radial | — | — |
| pharmaceutical | PCL17/5.0% VA64/3.0% | length/3.3 mm | | |
| ingredient- | P407/0.5% Vitamin E | triangular | | |
| containing | succinate/0.5% | cross-section | | |
| portion | | base length | | |
| | SiO₂/0.05% FD&C | | | |
| | Yellow 5 Alum lake | | | |
| | (16-18%)/0.05% FD&C | | | |
| | Blue 1 Alum lake | | | |
| | (11-13%) | | | |
| Fourth inert | 66.45% Corbion | 5.8 mm radial | 66.45% Corbion | 13.1 mm radial |
| segment | PC17/32.00% VA64/1.50% | length/3.1 mm | PC17/32.00% VA64/1.50% | length/3.1 mm |
| | P407/0.05% FD&C Blue 1 | triangular | P407/0.05% FD&C Blue 1 | triangular |
| | Alum lake (11-13%) | cross-section | Alum lake (11-13%) | cross-section |
| | | base length | | base length |

The drug arm was coated with 73.5% PCL17, 24.5% VA64, and 2.0% magnesium stearate, to form a release-rate modulating polymer film. The components were dissolved in ethyl acetate to prepare the coating. The ethyl acetate evaporated during the coating process. The coating was applied using a pan coater.

The arms of the gastric residence system were connected by a flexible line comprising polyglycolic acid (PGA). The flexible line was a Bondek size 3-0 braided, uncoated flexible line. The diameter of the flexible line was 0.2 mm. The flexible line was embedded in a distal end of each arm by laser welding.

The gastric residence system was folded and placed in a sleeve and capsule for administration. The sleeve was a size 0 HPMC capsule, cap only. The capsule was a size 00EL HPMC capsule. The capsule was coated with a coating comprising 90.7% by weight Eudragit E, 4.64% by weight dibutyl sebacate, and 4.64% by weight magnesium stearate.

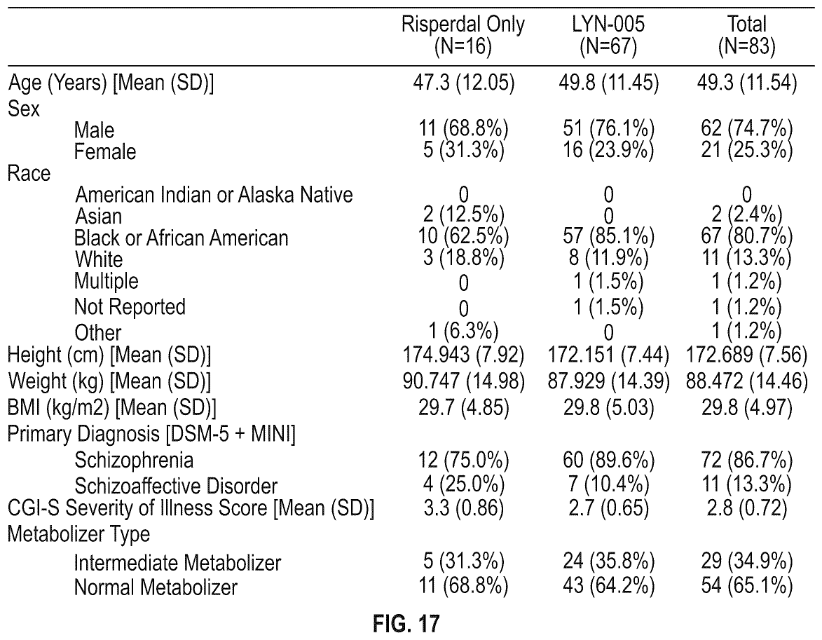

Performance of the gastric residence system (referred to as LYN-005) was tested in an open label, parallel group study on a group of clinically stable participants with schizophrenia or schizoaffective disorder on a stable dose of antipsychotic drug. Participants taking oral antipsychotics were switched to immediate release (IR) Risperdal for a 7-day run-in period before the start of week 1 of the study. Participants received a total daily 2 mg or 6 mg Risperdal (as 1 mg twice daily/2 mg once daily or as 3 mg twice daily/6 mg once daily, respectively) based on their current antipsychotic dose. Following the 7-day run-in period, participants received five doses of once-weekly LYN-005. During week 1, participants also received a supplemental daily half dose of the IR Risperdal received during the run-in period (1 mg or 3 mg, depending on the dosage received during the run-in period). [0335]83 subjects were enrolled in the study. 47 subjects completed the entire 5-week study. The number of subjects who dropped out after receiving only IR Risperdal (n=16 over 1 week) was similar to the number of subjects who dropped out while receiving LYN-005 (n=20 over 5 weeks). Most participants who dropped out did so during the 7-day run-in period before the start of week 1 (16/36) and during week 1 of the study (7/36). Demographics of the participants in the study are shown in FIG. 17.

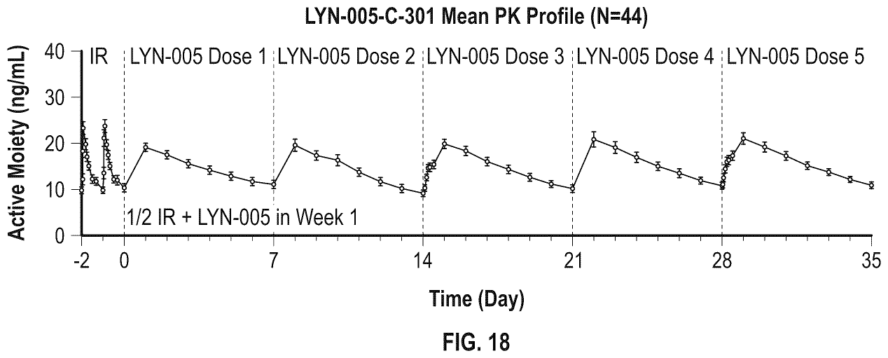

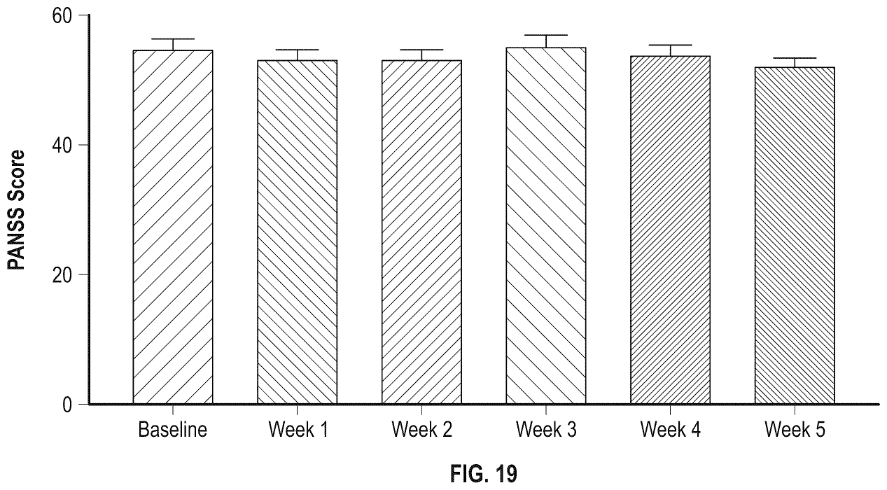

FIG. 18 shows a dose-normalized mean pharmacokinetic (PK) profile of the study participants (n=44) over the 5 week dosing period and the 7-day run-in period. The PK profile was normalized to a dose of 15 mg risperidone. Sustained release of risperidone was noted across all five doses. Drug concentrations were maintained above the therapeutic threshold across the dosing period. Drug concentrations were also well below the peak concentrations of immediate release Risperdal across all 5 doses of LYN-005.

Primary endpoints included comparison of Cmin, Cmax, and Cavg of LYN-005 at week 5 to that of IR Risperdal on the last day of run-in (Day −1). Primary endpoint criteria was determined to be a geometric mean ratio (GMR) with a 90% confidence interval (CI) of Cₘᵢₙ>0.80 for one-sided LYN/IR, GMR of Cmax<1.25 for one-sided LYN/IR, and GMR of Cavg within 0.80-1.40 for two-sided LYN/IR. All primary endpoint criteria was met, as shown in the table below.

| | | | |
|---|---|---|---|
| Primary | | 90% CI | 90% CI |
| Endpoint | Geometric | lower | upper |
| Criteria | Mean Ratio | limit | limit |

Week 5/Day −1

| | | | | |
|---|---|---|---|---|
| Cmin | One-sided | 1.02 | 0.86 | 1.22 |
| | LYN/IR > 0.80 | | | |
| Cmax | One-sided | 0.84 | 0.77 | 0.92 |
| | LYN/IR < 1.25 | | | |
| Cavg | Two-sided | 1.03 | 0.94 | 1.14 |
| | LYN/IR within | | | |
| | 0.80-1.40 | | | |

Secondary endpoints included change in Positive and Negative Syndrome Scale (PANSS) from baseline to week 5 and incidence of treatment emergent adverse events (TEALs) from week 1 to week 5. FIG. 19 shows average PANSS scores over the course of the study. The average PANSS score was maintained over the 5-week study period compared to the baseline. The PANSS scale measures the symptom severity of patients with schizophrenia.

The incidence of TEAEs was also measured. Of the 83 patients who received JR risperidone over the 7-day run-in period, 36 patients experienced any TEAL. None were serious adverse events (SAEs). Of the 67 patients who subsequently received at least one dose of LYN-on, 57 patients experienced any TEAL. One was an SAE. The TEAEs and SAEs are shown in the table below.

| | | |
|---|---|---|
| IR Run-in | LYN-005 | Overall |
| (n = 83) | (n = 67) | (n = 83) |

| | | | | | | |
|---|---|---|---|---|---|---|
| Any TEAE | 36 | (43.4%) | 57 | (85.1%) | 67 | (80.7%) |
| SAE | 0 | (0.0%) | 1 | (1.4%) | 1 | (1.2%) |
| TEAE leading to | 6 | (7.2%) | 9 | (13.4%) | 15 | (18.1%) |
| discontinuation | | | | | | |

TEAE occurring in >= 5% of participant population
in the LYN-005 group

| | | | | | | |
|---|---|---|---|---|---|---|
| Abdominal Pain | 1 | (1.2%) | 30 | (44.8%) | 31 | (37.3%) |
| GERD | 6 | (7.2%) | 10 | (14.9%) | 16 | (19.3%) |
| Constipation | 4 | (4.8%) | 8 | (11.9%) | 11 | (13.3%) |
| Dyspepsia | 3 | (3.6%) | 7 | (10.4%) | 10 | (12.0%) |
| Nausea | 1 | (1.2%) | 7 | (10.4%) | 7 | (8.4%) |
| Vomiting | 1 | (1.2%) | 8 | (11.9%) | 8 | (9.6%) |
| Headache | 8 | (9.6%) | 8 | (11.9%) | 15 | (18.1%) |
| Back Pain | 1 | (1.2%) | 6 | (9.0%) | 7 | (8.4%) |

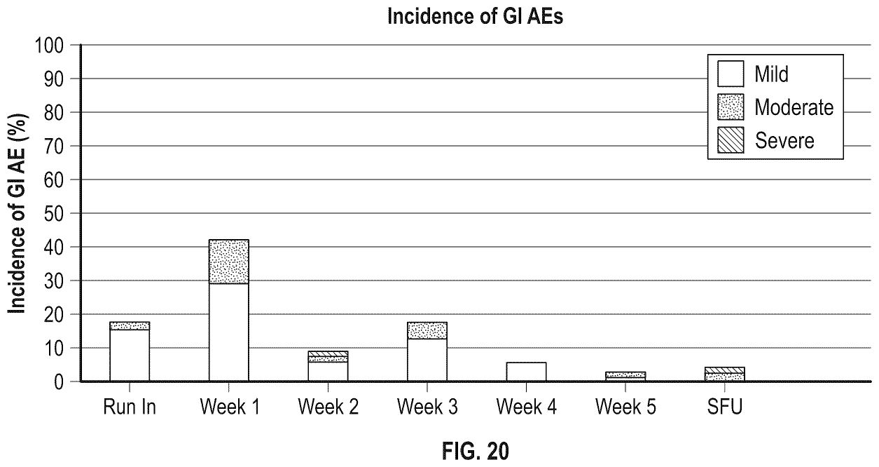

The majority of adverse events experienced were gastrointestinal adverse events (GI AEs). FIG. 20 shows the incidence of GI AEs over time for a group comprising 47 participants. As shown, the majority of GI AEs were mild (73%) and transient (less than 3 days in duration). The incidence of GI AEs was highest in week 1 of the study. GI AEs decreased over time, despite the presence of multiple stellates in the body during the later weeks of the study. The incidence rate in week 5 was over five times less than the incidence rate during the run-in period.

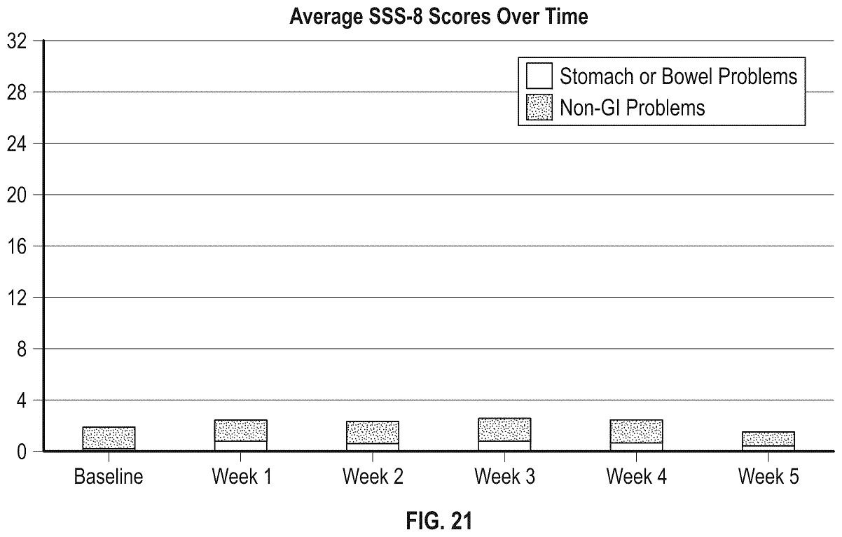

Subjects reported not being bothered by their GI AEs on the Somatic Symptom Scale (SSS-8). SSS-8 scores over time are shown in FIG. 21. SSS-8 scores had a weak correlation with the number of GI AEs reported during the same week (correlation coefficient=0.29). The presence of multiple stellates in a week did not correspond to higher scores on the SSS-8 scale.

### Exemplary Embodiments

Embodiment 1. A gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm, wherein the flexible line comprises an irregular cross-section.

Embodiment 2. The gastric residence system of embodiment 1, wherein the irregular cross-section comprises a braided cross-section.

Embodiment 3. The gastric residence system of embodiment 1 or 2, wherein the irregular cross-section comprises at least one of bumps, ridges, knots, barbs, or valleys.

Embodiment 4. The gastric residence system of any of embodiments 1-3, wherein the plurality of arms comprises at least three arms.

Embodiment 5. The gastric residence system of any of embodiments 1-4, wherein two or three arms comprise an active pharmaceutical ingredient-containing portion and a separate tip portion at a distal end of the two or three arms.

Embodiment 6. The gastric residence system of any of embodiments 1-5, wherein the active pharmaceutical ingredient comprises 10-80% by weight of the active pharmaceutical ingredient-containing portion.

Embodiment 7. The gastric residence system of any of embodiments 1-5, wherein the active pharmaceutical ingredient comprises 40-60% by weight of the active pharmaceutical ingredient-containing portion.

Embodiment 8. The gastric residence system of any of embodiments 1-5, wherein the active pharmaceutical ingredient-containing portion comprises 20-40% by weight of an active pharmaceutical ingredient.

Embodiment 9. The gastric residence system of any of embodiments 1-8, wherein each arm of the plurality of arms comprises a triangular cross-section.

Embodiment 10. The gastric residence system of embodiment 9, wherein the triangular cross-section has a base of 2.5-3.5 mm and a height of 2.1-3.1 mm.

Embodiment 11. The gastric residence system of embodiment 9, wherein the triangular cross-section has a base of 2.9-3.3 mm and a height of 2.5-2.9 mm.

Embodiment 12. The gastric residence system of any of embodiments 1-11, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

Embodiment 13. The gastric residence system of any of embodiments 1-11, wherein the flexible line comprises one or more of poly(glycolic acid) (PGA), poly(lactic-co-glycolic acid) (PLGA), or polyethylene terephthalate (PET).

Embodiment 14. The gastric residence system of any of embodiments 1-13, wherein the flexible line comprises PLGA in a poly(glycolic acid) (PGA) to poly(lactic acid) (PLA) ratio of 95:5 to 40:60.

Embodiment 15. The gastric residence system of any of embodiments 1-14, wherein the flexible line comprises PLGA with an intrinsic viscosity of 0.5-3 dL/g.

Embodiment 16. The gastric residence system of any of embodiments 1-15, wherein the flexible line comprises at least one crystalline material.

Embodiment 17. The gastric residence system of any of embodiments 1-16, wherein the flexible line comprises at least one semi-crystalline material.

Embodiment 18. The gastric residence system of any of embodiments 1-17, wherein the flexible line comprises at least one amorphous material.

Embodiment 19. The gastric residence system of any of embodiments 1-18, wherein the flexible line is configured to degrade in less than 14 days.

Embodiment 20. The gastric residence system of any of embodiments 1-18, wherein the flexible line is configured to degrade in less than 10 days.

Embodiment 21. The gastric residence system of any of embodiments 1-20, wherein the flexible line is a sterilized flexible line.

Embodiment 22. The gastric residence system of any of embodiments 1-21, wherein the flexible line has a diameter of 0.05-1 mm.

Embodiment 23. The gastric residence system of any of embodiments 1-22, wherein the flexible line has a cross-sectional area of 0.005-3.14 mm².

Embodiment 24. The gastric residence system of any of embodiments 1-22, wherein the flexible line has a cross-sectional area of 0.06-0.13 mm².

Embodiment 25. The gastric residence system of any of embodiments 1-22, wherein the flexible line has a cross-sectional area of 0.1-5 mm².

Embodiment 26. The gastric residence system of any of embodiments 1-22, wherein the flexible line has a cross-sectional area of 0.5-3.5 mm².

Embodiment 27. The gastric residence system of any of embodiments 1-22, wherein the flexible line has a cross-sectional area of 0.6-1 mm².

Embodiment 28. The gastric residence system of any of embodiments 1-27, wherein the flexible line has a surface roughness of 5-400 μm.

Embodiment 29. The gastric residence system of any of embodiments 1-27, wherein the flexible line has a surface roughness of 50-250 μm.

Embodiment 30. The gastric residence system of any of embodiments 1-29, wherein the flexible line comprises a braid of at least three strands.

Embodiment 31. The gastric residence system of embodiment 30, wherein the braid has a braid angle of 5-40 degrees and a braid length of 0.5-3.1 mm.

Embodiment 32. The gastric residence system of any of embodiments 1-31, wherein the flexible line is absorbable.

Embodiment 33. The gastric residence system of any of embodiments 1-31, wherein the flexible line is non-absorbable.

Embodiment 34. The gastric residence system of any of embodiments 1-33, wherein the flexible line comprises a coating.

Embodiment 35. The gastric residence system of embodiment 34, wherein the coating comprises polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

Embodiment 36. The gastric residence system of any of embodiments 1-35, wherein the separate tip portion comprises polycaprolactone.

Embodiment 37. The gastric residence system of embodiment 36, wherein the separate tip portion further comprises copovidone.

Embodiment 38. The gastric residence system of any of embodiments 36-37, wherein the separate tip portion further comprises Poloxamer 407.

Embodiment 39. The gastric residence system of any of embodiments 36-38, wherein the separate tip portion further comprises a colorant.

Embodiment 40. The gastric residence system of any of embodiments 36-39, wherein the separate tip portion further comprises bismuth subcarbonate.

Embodiment 41. The gastric residence system of any of embodiments 1-40, wherein the distal end of each arm comprises polycaprolactone.

Embodiment 42. The gastric residence system of embodiment 41, wherein the distal end of each arm further comprises copovidone.

Embodiment 43. The gastric residence system of any of embodiments 41-42, wherein the distal end of each arm further comprises Poloxamer 407.

Embodiment 44. The gastric residence system of any of embodiments 41-43, wherein the distal end of each arm further comprises a colorant.

Embodiment 45. The gastric residence system of any of embodiments 41-44, wherein the distal end of each arm further comprises bismuth subcarbonate.

Embodiment 46. The gastric residence system of any of embodiments 1-45, wherein the separate tip portion has a flexural modulus of at least 50 MPa.

Embodiment 47. The gastric residence system of any of embodiments 1-46, wherein the distal end of each arm has a flexural modulus of at least 50 MPa.

Embodiment 48. The gastric residence system of any of embodiments 1-47, wherein the separate tip portion has a flexural strength of at least 5 N.

Embodiment 49. The gastric residence system of any of embodiments 1-48, wherein the separate tip portion has a flexural strength of at least 8 N.

Embodiment 50. The gastric residence system of any of embodiments 1-49, wherein the distal end of each arm has a flexural strength of at least 5 N.

Embodiment 51. The gastric residence system of any of embodiments 1-50, wherein the distal end of each arm has a flexural strength of at least 8 N.

Embodiment 52. The gastric residence system of any of embodiments 1-51, wherein the separate tip portion has a maximum force of at least 3 N.

Embodiment 53. The gastric residence system of any of embodiments 1-52, wherein the separate tip portion has a maximum force of at least 5 N.

Embodiment 54. The gastric residence system of any of embodiments 1-53, wherein the distal end of each arm has a maximum force of at least 3 N.

Embodiment 55. The gastric residence system of any of embodiments 1-54, wherein the distal end of each arm has a maximum force of at least 5 N.

Embodiment 56. The gastric residence system of any of embodiments 1-55, wherein the separate tip portion softens less than 80% upon hydration.

Embodiment 57. The gastric residence system of any of embodiments 1-56, wherein the separate tip portion softens more than 10% upon hydration.

Embodiment 58. The gastric residence system of any of embodiments 1-57, wherein the distal end of each arm softens less than 80% upon hydration.

Embodiment 59. The gastric residence system of any of embodiments 1-58, wherein the distal end of each arm softens more than 10% upon hydration.

Embodiment 60. The gastric residence system of any of embodiments 1-59, wherein the flexible line is embedded in the distal end of each arm by heating the distal end, pressing a portion of the flexible line into the distal end, and cooling the distal end with the portion of the flexible line embedded.

Embodiment 61. The gastric residence system of embodiment 60, wherein heating the distal end comprises directing a laser at the distal end.

Embodiment 62. The gastric residence system of embodiment 61, wherein the laser comprises an infrared laser.

Embodiment 63. The gastric residence system of any of embodiments 60-62, wherein the heating comprises convection heating.

Embodiment 64. The gastric residence system of any of embodiments 1-63, wherein a force required to release the flexible line from a first arm of the plurality of arms is at least 4N.

Embodiment 65. The gastric residence system of any of embodiments 1-64, wherein a force required to release the flexible line from a first arm of the plurality of arms is at least 8N.

Embodiment 66. The gastric residence system of any of embodiments 1-65, wherein the gastric residence system is configured to be folded during administration and is configured to assume an open configuration when in a patient's stomach.

Embodiment 67. The gastric residence system of any of embodiments 1-66, wherein the gastric residence system has a multi-armed star shape in the open configuration.

Embodiment 68. The gastric residence system of any of embodiments 1-67, comprising a core, wherein each arm of the plurality of arms is connected to the core at the proximal end of each arm.

Embodiment 69. The gastric residence system of embodiment 68, wherein the core undergoes elastic deformation when the gastric residence system is in the folded configuration and recoils when the gastric residence system assumes the open configuration.

Embodiment 70. The gastric residence system of embodiment 68 or 69, comprising a plurality of linker components, wherein one linker component of the plurality of linker components connects one arm of the plurality of arms to the core.

Embodiment 71. The gastric residence system of embodiment 70, wherein each linker component of the plurality of linker components degrades, dissolves, disassociates, or mechanically weakens in a gastric environment.

Embodiment 72. The gastric residence system of any of embodiments 1-71, wherein the gastric residence system is used to treat a patient.

Embodiment 73. The gastric residence system of embodiment 72, wherein the patient is a human.

Embodiment 74. A gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

Embodiment 75. The gastric residence system of embodiment 74, wherein the flexible line comprises an irregular cross-section.

Embodiment 76. The gastric residence system of embodiment 74 or 75, wherein the irregular cross-section comprises a braided cross-section.

Embodiment 77. The gastric residence system of any of embodiments 74-76, wherein the irregular cross-section comprises at least one of bumps, ridges, knots, barbs, or valleys.

Embodiment 78. The gastric residence system of any of embodiments 74-77, wherein the plurality of arms comprises at least three arms.

Embodiment 79. The gastric residence system of any of embodiments 74-78, wherein two or three arms comprise an active pharmaceutical ingredient-containing portion and a separate tip portion at a distal end of the two or three arms.

Embodiment 80. The gastric residence system of any of embodiments 74-79, wherein the active pharmaceutical ingredient-containing portion comprises 10-80% by weight of an active pharmaceutical ingredient.

Embodiment 81. The gastric residence system of any of embodiments 74-80, wherein the active pharmaceutical ingredient-containing portion comprises 40-60% by weight of an active pharmaceutical ingredient.

Embodiment 82. The gastric residence system of any of embodiments 74-81, wherein the active pharmaceutical ingredient-containing portion comprises 20-40% by weight of an active pharmaceutical ingredient.

Embodiment 83. The gastric residence system of any of embodiments 74-82, wherein each arm of the plurality of arms comprises a triangular cross-section.

Embodiment 84. The gastric residence system of embodiment 83, wherein the triangular cross-section has a base of 2.5-3.5 mm and a height of 2.1-3.1 mm.

Embodiment 85. The gastric residence system of embodiment 83, wherein the triangular cross-section has a base of 2.9-3.3 mm and a height of 2.5-2.9 mm.

Embodiment 86. The gastric residence system of any of embodiments 74-85, wherein the flexible line comprises PLGA in a poly(glycolic acid) (PGA) to poly(lactic acid) (PLA) ratio of 95:5 to 40:60.

Embodiment 87. The gastric residence system of any of embodiments 74-86, wherein the flexible line comprises PLGA with an intrinsic viscosity of 0.5-3 dL/g.

Embodiment 88. The gastric residence system of any of embodiments 74-87, wherein the flexible line comprises at least one crystalline material.

Embodiment 89. The gastric residence system of any of embodiments 74-88, wherein the flexible line comprises at least one semi-crystalline material.

Embodiment 90. The gastric residence system of any of embodiments 74-89, wherein the flexible line comprises at least one amorphous material.

Embodiment 91. The gastric residence system of any of embodiments 74-90, wherein the flexible line is configured to degrade in less than 14 days.

Embodiment 92. The gastric residence system of any of embodiments 74-91, wherein the flexible line is configured to degrade in less than 10 days.

Embodiment 93. The gastric residence system of any of embodiments 74-92, wherein the flexible line is a sterilized flexible line.

Embodiment 94. The gastric residence system of any of embodiments 74-93, wherein the flexible line has a diameter of 0.05-1 mm.

Embodiment 95. The gastric residence system of any of embodiments 74-94, wherein the flexible line has a cross-sectional area of 0.005-3.14 mm².

Embodiment 96. The gastric residence system of any of embodiments 74-94, wherein the flexible line has a cross-sectional area of 0.06-0.13 mm².

Embodiment 97. The gastric residence system of any of embodiments 74-94, wherein the flexible line has a cross-sectional area of 0.1-5 mm².

Embodiment 98. The gastric residence system of any of embodiments 74-94, wherein the flexible line has a cross-sectional area of 0.5-3.5 mm².

Embodiment 99. The gastric residence system of any of embodiments 74-94, wherein the flexible line has a cross-sectional area of 0.6-1 mm².

Embodiment 100. The gastric residence system of any of embodiments 74-99, wherein the flexible line has a surface roughness of 5-400 μm.

Embodiment 101. The gastric residence system of any of embodiments 74-99, wherein the flexible line has a surface roughness of 50-250 μm.

Embodiment 102. The gastric residence system of any of embodiments 74-101, wherein the flexible line comprises a braid of at least three strands.

Embodiment 103. The gastric residence system of embodiment 102, wherein the braid has a braid angle of 5-40 degrees and a braid length of 0.5-3.1 mm.

Embodiment 104. The gastric residence system of any of embodiments 74-103, wherein the flexible line is absorbable.

Embodiment 105. The gastric residence system of any of embodiments 74-103, wherein the flexible line is non-absorbable.

Embodiment 106. The gastric residence system of any of embodiments 74-105, wherein the flexible line comprises a coating.

Embodiment 107. The gastric residence system of embodiment 106, wherein the coating comprises polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

Embodiment 108. The gastric residence system of any of embodiments 74-107, wherein the separate tip portion comprises polycaprolactone.

Embodiment 109. The gastric residence system of embodiment 108, wherein the separate tip portion further comprises copovidone.

Embodiment 110. The gastric residence system of any of embodiments 108-109, wherein the separate tip portion further comprises Poloxamer 407.

Embodiment 111. The gastric residence system of any of embodiments 108-110 wherein the separate tip portion further comprises a colorant.

Embodiment 112. The gastric residence system of any of embodiments 108-111, wherein the separate tip portion further comprises bismuth subcarbonate.

Embodiment 113. The gastric residence system of any of embodiments 74-112, wherein the distal end of each arm comprises polycaprolactone.

Embodiment 114. The gastric residence system of embodiment 113, wherein the distal end of each arm further comprises copovidone.

Embodiment 115. The gastric residence system of any of embodiments 113-114, wherein the distal end of each arm further comprises Poloxamer 407.

Embodiment 116. The gastric residence system of any of embodiments 113-115, wherein the distal end of each arm further comprises a colorant.

Embodiment 117. The gastric residence system of any of embodiments 113-116, wherein the distal end of each arm further comprises bismuth subcarbonate.

Embodiment 118. The gastric residence system of any of embodiments 74-117, wherein the separate tip portion has a flexural modulus of at least 50 MPa.

Embodiment 119. The gastric residence system of any of embodiments 74-118, wherein the distal end of each arm has a flexural modulus of at least 50 MPa.

Embodiment 120. The gastric residence system of any of embodiments 74-119, wherein the separate tip portion has a flexural strength of at least 5 N.

Embodiment 121. The gastric residence system of any of embodiments 74-120, wherein the separate tip portion has a flexural strength of at least 8 N.

Embodiment 122. The gastric residence system of any of embodiments 74-121, wherein the distal end of each arm has a flexural strength of at least 5 N.

Embodiment 123. The gastric residence system of any of embodiments 74-122, wherein the distal end of each arm has a flexural strength of at least 8 N.

Embodiment 124. The gastric residence system of any of embodiments 74-123, wherein the separate tip portion has a maximum force of at least 3 N.

Embodiment 125. The gastric residence system of any of embodiments 74-124, wherein the separate tip portion has a maximum force of at least 5 N.

Embodiment 126. The gastric residence system of any of embodiments 74-125, wherein the distal end of each arm has a maximum force of at least 3 N.

Embodiment 127. The gastric residence system of any of embodiments 74-126, wherein the distal end of each arm has a maximum force of at least 5 N.

Embodiment 128. The gastric residence system of any of embodiments 74-127, wherein the separate tip portion softens less than 80% upon hydration.

Embodiment 129. The gastric residence system of any of embodiments 74-128, wherein the separate tip portion softens more than 10% upon hydration.

Embodiment 130. The gastric residence system of any of embodiments 74-129, wherein the distal end of each arm softens less than 80% upon hydration.

Embodiment 131. The gastric residence system of any of embodiments 74-130, wherein the distal end of each arm softens more than 10% upon hydration.

Embodiment 132. The gastric residence system of any of embodiments 74-131, wherein the flexible line is embedded in the distal end of each arm by heating the distal end, pressing a portion of the flexible line into the distal end, and cooling the distal end with the portion of the flexible line embedded.

Embodiment 133. The gastric residence system of embodiment 132, wherein heating the distal end comprises directing a laser at the distal end.

Embodiment 134. The gastric residence system of embodiment 133, wherein the laser comprises an infrared laser.

Embodiment 135. The gastric residence system of any of embodiments 132-134, wherein the heating comprises convection heating.

Embodiment 136. The gastric residence system of any of embodiments 74-135, wherein a force required to release the flexible line from a first arm of the plurality of arms is at least 4N.

Embodiment 137. The gastric residence system of any of embodiments 74-136, wherein a force required to release the flexible line from a first arm of the plurality of arms is at least 8N.

Embodiment 138. The gastric residence system of any of embodiments 74-137, wherein the gastric residence system is configured to be folded during administration and is configured to assume an open configuration when in a patient's stomach.

Embodiment 139. The gastric residence system of any of embodiments 74-138wherein the gastric residence system has a multi-armed star shape in the open configuration.

Embodiment 140. The gastric residence system of any of embodiments 74-139, comprising a core, wherein each arm of the plurality of arms is connected to the core at the proximal end of each arm.

Embodiment 141. The gastric residence system of embodiment 140, wherein the core undergoes elastic deformation when the gastric residence system is in the folded configuration and recoils when the gastric residence system assumes the open configuration.

Embodiment 142. The gastric residence system of embodiment 140 or 141, comprising a plurality of linker components, wherein one linker component of the plurality of linker components connects one arm of the plurality of arms to the core.

Embodiment 143. The gastric residence system of embodiment 142, wherein each linker component of the plurality of linker components degrades, dissolves, disassociates, or mechanically weakens in a gastric environment.

Embodiment 144. The gastric residence system of any of embodiments 74-143, wherein the gastric residence system is used to treat a patient.

Embodiment 145. The gastric residence system of embodiment 144, wherein the patient is a human.

Embodiment 146. A gastric residence system comprising: a plurality of arms connected at a proximal end of each arm, the plurality of arms extending radially from the proximal ends, at least one arm comprising an active pharmaceutical ingredient-containing portion; and a flexible line connecting the plurality of arms, wherein a respective portion of the flexible line is embedded within a distal end of each arm, wherein the flexible line comprises an irregular cross-section.

Embodiment 147. The gastric residence system of embodiment 146, wherein the active pharmaceutical ingredient-containing portion comprises 10-80% by weight of an active pharmaceutical ingredient.

Embodiment 148. The gastric residence system of embodiment 146, wherein the active pharmaceutical ingredient-containing portion comprises 40-60% by weight of an active pharmaceutical ingredient.

Embodiment 149. The gastric residence system of embodiment 146, wherein the active pharmaceutical ingredient-containing portion comprises 20-40% by weight of an active pharmaceutical ingredient.

Embodiment 150. The gastric residence system of any of embodiments 146-149, wherein the irregular cross-section comprises a braided cross-section.

Embodiment 151. The gastric residence system of any of embodiments 146-150, wherein the irregular cross-section comprises at least one of bumps, ridges, knots, barbs, or valleys.

Embodiment 152. The gastric residence system of any of embodiments 146-151, wherein the plurality of arms comprises at least three arms.

Embodiment 153. The gastric residence system of any of embodiments 146-152, wherein each arm of the plurality of arms comprises a triangular cross-section.

Embodiment 154. The gastric residence system of embodiment 153, wherein the triangular cross-section has a base of 2.5-3.5 mm and a height of 2.1-3.1 mm.

Embodiment 155. The gastric residence system of embodiment 153, wherein the triangular cross-section has a base of 2.9-3.3 mm and a height of 2.5-2.9 mm.

Embodiment 156. The gastric residence system of any of embodiments 146-155, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

Embodiment 157. The gastric residence system of any of embodiments 146-156, wherein the flexible line comprises one or more of poly(glycolic acid) (PGA), poly(lactic-co-glycolic acid) (PLGA), or polyethylene terephthalate (PET).

Embodiment 158. The gastric residence system of any of embodiments 146-157, wherein the flexible line comprises PLGA in a poly(glycolic acid) (PGA) to poly(lactic acid) (PLA) ratio of 95:5 to 40:60.

Embodiment 159. The gastric residence system of any of embodiments 146-158, wherein the flexible line comprises PLGA with an intrinsic viscosity of 0.5-3 dL/g.

Embodiment 160. The gastric residence system of any of embodiments 146-159, wherein the flexible line comprises at least one crystalline material.

Embodiment 161. The gastric residence system of any of embodiments 146-160, wherein the flexible line comprises at least one semi-crystalline material.

Embodiment 162. The gastric residence system of any of embodiments 146-161, wherein the flexible line comprises at least one amorphous material.

Embodiment 163. The gastric residence system of any of embodiments 146-162, wherein the flexible line is configured to degrade in less than 14 days.

Embodiment 164. The gastric residence system of any of embodiments 146-163, wherein the flexible line is configured to degrade in less than 10 days.

Embodiment 165. The gastric residence system of any of embodiments 146-164, wherein the flexible line is a sterilized flexible line.

Embodiment 166. The gastric residence system of any of embodiments 146-165, wherein the flexible line has a diameter of 0.05-1 mm.

Embodiment 167. The gastric residence system of any of embodiments 146-166, wherein the flexible line has a cross-sectional area of 0.005-3.14 mm².

Embodiment 168. The gastric residence system of any of embodiments 146-166, wherein the flexible line has a cross-sectional area of 0.06-0.13 mm².

Embodiment 169. The gastric residence system of any of embodiments 146-166, wherein the flexible line has a cross-sectional area of 0.1-5 mm².

Embodiment 170. The gastric residence system of any of embodiments 146-166, wherein the flexible line has a cross-sectional area of 0.5-3.5 mm².

Embodiment 171. The gastric residence system of any of embodiments 146-166, wherein the flexible line has a cross-sectional area of 0.6-1 mm².

Embodiment 172. The gastric residence system of any of embodiments 146-171, wherein the flexible line has a surface roughness of 5-400 μm.

Embodiment 173. The gastric residence system of any of embodiments 146-171, wherein the flexible line has a surface roughness of 50-250 μm.

Embodiment 174. The gastric residence system of any of embodiments 146-173, wherein the flexible line comprises a braid of at least three strands.

Embodiment 175. The gastric residence system of embodiment 174, wherein the braid has a braid angle of 5-40 degrees and a braid length of 0.5-3.1 mm.

Embodiment 176. The gastric residence system of any of embodiments 146-175, wherein the flexible line is absorbable.

Embodiment 177. The gastric residence system of any of embodiments 146-175, wherein the flexible line is non-absorbable.

Embodiment 178. The gastric residence system of any of embodiments 146-177, wherein the flexible line comprises a coating.

Embodiment 179. The gastric residence system of embodiment 178, wherein the coating comprises polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

Embodiment 180. The gastric residence system of any of embodiments 146-179, wherein the distal end of each arm comprises polycaprolactone.

Embodiment 181. The gastric residence system of embodiment 180, wherein the distal end of each arm further comprises copovidone.

Embodiment 182. The gastric residence system of any of embodiments 180-181, wherein the distal end of each arm further comprises Poloxamer 407.

Embodiment 183. The gastric residence system of any of embodiments 180-182, wherein the distal end of each arm further comprises a colorant.

Embodiment 184. The gastric residence system of any of embodiments 180-183, wherein the distal end of each arm further comprises bismuth subcarbonate.

Embodiment 185. The gastric residence system of any of embodiments 146-184, wherein the distal end of each arm has a flexural modulus of at least 50 MPa.

Embodiment 186. The gastric residence system of any of embodiments 146-185, wherein the distal end of each arm has a flexural strength of at least 5 N.

Embodiment 187. The gastric residence system of any of embodiments 146-186, wherein the distal end of each arm has a flexural strength of at least 8 N.

Embodiment 188. The gastric residence system of any of embodiments 146-187, wherein the distal end of each arm has a maximum force of at least 3 N.

Embodiment 189. The gastric residence system of any of embodiments 146-188, wherein the distal end of each arm has a maximum force of at least 5 N.

Embodiment 190. The gastric residence system of any of embodiments 146-189, wherein the distal end of each arm softens less than 80% upon hydration.

Embodiment 191. The gastric residence system of any of embodiments 146-190, wherein the distal end of each arm softens more than 10% upon hydration.

Embodiment 192. The gastric residence system of any of embodiments 146-191, wherein the at least one arm comprising an active pharmaceutical ingredient-containing portion comprises a separate tip portion at a distal end of the at least one arm.

Embodiment 193. The gastric residence system of any of embodiments 146-192, wherein two or three arms comprise an active pharmaceutical ingredient-containing portion and a separate tip portion at a distal end of the two or three arms.

Embodiment 194. The gastric residence system of embodiment 192 or 193, wherein the separate tip portion comprises polycaprolactone.

Embodiment 195. The gastric residence system of embodiment 194, wherein the separate tip portion further comprises copovidone.

Embodiment 196. The gastric residence system of any of embodiments 194-195, wherein the separate tip portion further comprises Poloxamer 407.

Embodiment 197. The gastric residence system of any of embodiments 194-196, wherein the separate tip portion further comprises a colorant.

Embodiment 198. The gastric residence system of any of embodiments 194-197, wherein the separate tip portion further comprises bismuth subcarbonate.

Embodiment 199. The gastric residence system of any of embodiments 192-198, wherein the separate tip portion has a flexural modulus of at least 50 MPa.

Embodiment 200. The gastric residence system of any of embodiments 192-199, wherein the separate tip portion has a flexural strength of at least 5 N.

Embodiment 201. The gastric residence system of any of embodiments 192-200, wherein the separate tip portion has a flexural strength of at least 8 N.

Embodiment 202. The gastric residence system of any of embodiments 192-201, wherein the separate tip portion has a maximum force of at least 3 N.

Embodiment 203. The gastric residence system of any of embodiments 192-202, wherein the separate tip portion has a maximum force of at least 5 N.

Embodiment 204. The gastric residence system of any of embodiments 192-203, wherein the separate tip portion softens less than 80% upon hydration.

Embodiment 205. The gastric residence system of any of embodiments 192-204, wherein the separate tip portion softens more than 10% upon hydration.

Embodiment 206. The gastric residence system of any of embodiments 146-205, wherein the flexible line is embedded in the distal end of each arm by heating the distal end, pressing a portion of the flexible line into the distal end, and cooling the distal end with the portion of the flexible line embedded.

Embodiment 207. The gastric residence system of embodiment 206, wherein heating the distal end comprises directing a laser at the distal end.

Embodiment 208. The gastric residence system of embodiment 207, wherein the laser comprises an infrared laser.

Embodiment 209. The gastric residence system of any of embodiments 206-208, wherein the heating comprises convection heating.

Embodiment 210. The gastric residence system of any of embodiments 146-209, wherein a force required to release the flexible line from a first arm of the plurality of arms is at least 4N.

Embodiment 211. The gastric residence system of any of embodiments 146-210, wherein a force required to release the flexible line from a first arm of the plurality of arms is at least 8N.

Embodiment 212. The gastric residence system of any of embodiments 146-211, wherein the gastric residence system is configured to be folded during administration and is configured to assume an open configuration when in a patient's stomach.

Embodiment 213. The gastric residence system of any of embodiments 146-212, wherein the gastric residence system has a multi-armed star shape in the open configuration.

Embodiment 214. The gastric residence system of any of embodiments 146-213, comprising a core, wherein each arm of the plurality of arms is connected to the core at the proximal end of each arm.

Embodiment 215. The gastric residence system of embodiment 214, wherein the core undergoes elastic deformation when the gastric residence system is in the folded configuration and recoils when the gastric residence system assumes the open configuration.

Embodiment 216. The gastric residence system of embodiment 214 or 215, comprising a plurality of linker components, wherein one linker component of the plurality of linker components connects one arm of the plurality of arms to the core.

Embodiment 217. The gastric residence system of embodiment 216, wherein each linker component of the plurality of linker components degrades, dissolves, disassociates, or mechanically weakens in a gastric environment.

Embodiment 218. The gastric residence system of any of embodiments 146-217, wherein the gastric residence system is used to treat a patient.

Embodiment 219. The gastric residence system of embodiment 218, wherein the patient is a human.

Embodiment 220. A method for manufacturing a gastric residence system comprising: using a mold, preparing a gastric residence system comprising a plurality of arms connected at a proximal end of each arm, the plurality of arms extending radially from the proximal ends, at least one arm comprising an active pharmaceutical ingredient-containing portion and a separate tip portion at a distal end of the at least one arm; heating the distal end of each arm to at least partially melt the respective distal end; embedding a respective portion of at least one flexible line into each melted distal end; and cooling the melted distal ends such that melted distal ends solidify with the at least one flexible line embedded.

Embodiment 221. A method for manufacturing a gastric residence system comprising: using a mold, preparing a gastric residence system comprising a plurality of arms connected at a proximal end of each arm, the plurality of arms extending radially from the proximal ends; heating the distal end of each arm to at least partially melt the respective distal end; embedding a respective portion of at least one flexible line into each melted distal end; and cooling the melted distal ends such that melted distal ends solidify with the at least one flexible line embedded.

Embodiment 222. The method of embodiment 221, wherein heating the distal end of each arm comprises directing a laser at the distal end of each arm.

Embodiment 223. The method of embodiment 222, wherein the laser comprises an infrared laser.

Embodiment 224. The method of any of embodiments 221-223, wherein the heating comprises convection heating.

Embodiment 225. The method of any of embodiments 221-224, wherein the flexible line comprises an irregular cross-section.

Embodiment 226. The method of any of embodiments 221-225, wherein the irregular cross-section comprises a braided cross-section.

Embodiment 227. The method of any of embodiments 221-226, wherein the irregular cross-section comprises at least one of bumps, ridges, knots, barbs, or valleys.

Embodiment 228. The method of any of embodiments 221-227, wherein the plurality of arms comprises at least three arms.

Embodiment 229. The method of any of embodiments 221-228, wherein two or three arms comprise an active pharmaceutical ingredient-containing portion and a separate tip portion at a distal end of the two or three arms.

Embodiment 230. The method of any of embodiments 221-229, wherein the active pharmaceutical ingredient-containing portion comprises 10-80% by weight of an active pharmaceutical ingredient.

Embodiment 231. The method of any of embodiments 221-229, wherein the active pharmaceutical ingredient-containing portion comprises 40-60% by weight of an active pharmaceutical ingredient.

Embodiment 232. The method of any of embodiments 221-229, wherein the active pharmaceutical ingredient-containing portion comprises 20-40% by weight of an active pharmaceutical ingredient.

Embodiment 233. The method of any of embodiments 221-232, wherein each arm of the plurality of arms comprises a triangular cross-section.

Embodiment 234. The method of embodiment 233, wherein the triangular cross-section has a base of 2.5-3.5 mm and a height of 2.1-3.1 mm.

Embodiment 235. The method of embodiment 233, wherein the triangular cross-section has a base of 2.9-3.3 mm and a height of 2.5-2.9 mm.

Embodiment 236. The method of any of embodiments 221-235, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

Embodiment 237. The method of any of embodiments 221-236, wherein the flexible line comprises one or more of polyglycolic acid, poly(lactic-co-glycolic acid), or polyethylene terephthalate.

Embodiment 238. The method of any of embodiments 221-237 wherein the flexible line comprises PLGA in a poly(glycolic acid) (PGA) to poly(lactic acid) (PLA) ratio of 95:5 to 40:60.

Embodiment 239. The method of any of embodiments 221-238, wherein the flexible line comprises PLGA with an intrinsic viscosity of 0.5-3 dL/g.

Embodiment 240. The method of any of embodiments 221-239, wherein the flexible line comprises at least one crystalline material.

Embodiment 241. The method of any of embodiments 221-240, wherein the flexible line comprises at least one semi-crystalline material.

Embodiment 242. The method of any of embodiments 221-241, wherein the flexible line comprises at least one amorphous material.

Embodiment 243. The method of any of embodiments 221-242, wherein the flexible line is configured to degrade in less than 14 days.

Embodiment 244. The method of any of embodiments 221-243, wherein the flexible line is configured to degrade in less than 10 days.

Embodiment 245. The method of any of embodiments 221-244, wherein the flexible line is a sterilized flexible line.

Embodiment 246. The method of any of embodiments 221-245, wherein the flexible line has a diameter of 0.05-1 mm.

Embodiment 247. The method of any of embodiments 221-246, wherein the flexible line has a cross-sectional area of 0.005-3.14 mm².

Embodiment 248. The method of any of embodiments 221-246, wherein the flexible line has a cross-sectional area of 0.06-0.13 mm².

Embodiment 249. The method of any of embodiments 221-246, wherein the flexible line has a cross-sectional area of 0.1-5 mm².

Embodiment 250. The method of any of embodiments 221-246, wherein the flexible line has a cross-sectional area of 0.5-3.5 mm².

Embodiment 251. The method of any of embodiments 221-246, wherein the flexible line has a cross-sectional area of 0.6-1 mm².

Embodiment 252. The method of any of embodiments 221-251, wherein the flexible line has a surface roughness of 5-400 μm.

Embodiment 253. The method of any of embodiments 221-251, wherein the flexible line has a surface roughness of 50-250 μm.

Embodiment 254. The method of any of embodiments 221-253, wherein the flexible line comprises a braid of at least three strands.

Embodiment 255. The method of embodiment 254, wherein the braid has a braid angle of 5-40 degrees and a braid length of 0.5-3.1 mm.

Embodiment 256. The method of any of embodiments 221-255, wherein the flexible line is absorbable.

Embodiment 257. The method of any of embodiments 221-255, wherein the flexible line is non-absorbable.

Embodiment 258. The method of any of embodiments 221-257, wherein the flexible line comprises a coating.

Embodiment 259. The method of embodiment 258, wherein the coating comprises polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

Embodiment 260. The method of any of embodiments 221-259, wherein the separate tip portion comprises polycaprolactone.

Embodiment 261. The method of embodiment 260, wherein the separate tip portion further comprises copovidone.

Embodiment 262. The method of any of embodiments 260-261, wherein the separate tip portion further comprises poloxamer 407.

Embodiment 263. The method of any of embodiments 260-262, wherein the separate tip portion further comprises a colorant.

Embodiment 264. The method of any of embodiments 260-263, wherein the separate tip portion further comprises bismuth subcarbonate.

Embodiment 265. The method of any of embodiments 221-264, wherein the distal end of each arm comprises polycaprolactone.

Embodiment 266. The method of embodiment 265, wherein the distal end of each arm further comprises copovidone.

Embodiment 267. The method of any of embodiments 265-266, wherein the distal end of each arm further comprises Poloxamer 407.

Embodiment 268. The method of any of embodiments 265-267, wherein the distal end of each arm further comprises a colorant.

Embodiment 269. The method of any of embodiments 265-268, wherein the distal end of each arm further comprises bismuth subcarbonate.

Embodiment 270. The method of any of embodiments 221-269, wherein the separate tip portion has an elastic modulus of at least 50 MPa.

Embodiment 271. The method of any of embodiments 221-270, wherein the distal end of each arm has an elastic modulus of at least 50 MPa.

Embodiment 272. The method of any of embodiments 221-271, wherein the distal end of each arm has a flexural modulus of at least 50 MPa.

Embodiment 273. The method of any of embodiments 221-272, wherein the separate tip portion has a flexural strength of at least 5 N.

Embodiment 274. The method of any of embodiments 221-273, wherein the separate tip portion has a flexural strength of at least 8 N.

Embodiment 275. The method of any of embodiments 221-274, wherein the distal end of each arm has a flexural strength of at least 5 N.

Embodiment 276. The method of any of embodiments 221-275, wherein the distal end of each arm has a flexural strength of at least 8 N.

Embodiment 277. The method of any of embodiments 221-276, wherein the separate tip portion has a maximum force of at least 3 N.

Embodiment 278. The method of any of embodiments 221-277, wherein the separate tip portion has a maximum force of at least 5 N.

Embodiment 279. The method of any of embodiments 221-278, wherein the distal end of each arm has a maximum force of at least 3 N.

Embodiment 280. The method of any of embodiments 221-279, wherein the distal end of each arm has a maximum force of at least 5 N.

Embodiment 281. The method of any of embodiments 221-280, wherein the separate tip portion softens less than 80% upon hydration.

Embodiment 282. The method of any of embodiments 221-281, wherein the separate tip portion softens more than 10% upon hydration.

Embodiment 283. The method of any of embodiments 221-282, wherein the distal end of each arm softens less than 80% upon hydration.

Embodiment 284. The method of any of embodiments 221-283, wherein the distal end of each arm softens more than 10% upon hydration.

Embodiment 285. The method of any of embodiments 221-284, wherein a force required to release the flexible line from a first arm of the plurality of arms is at least 4N.

Embodiment 286. The method of any of embodiments 221-285, wherein a force required to release the flexible line from a first arm of the plurality of arms is at least 8N.

Embodiment 287. The method of any of embodiments 221-286, wherein the gastric residence system is configured to be folded during administration and is configured to assume an open configuration when in a patient's stomach.

Embodiment 288. The method of any of embodiments 221-287, wherein the gastric residence system has a multi-armed star shape in the open configuration.

Embodiment 289. The method of any of embodiments 221-288, wherein the gastric residence system comprises a core, wherein each arm of the plurality of arms is connected to the core at the proximal end of each arm.

Embodiment 290. The method of embodiment 289, wherein the core undergoes elastic deformation when the gastric residence system is in the folded configuration and recoils when the gastric residence system assumes the open configuration.

Embodiment 291. The method of embodiment 289 or 290, wherein the gastric residence system comprises a plurality of linker components, wherein one linker component of the plurality of linker components connects one arm of the plurality of arms to the core.

Embodiment 292. The method of embodiment 291, wherein each linker component of the plurality of linker components degrades, dissolves, disassociates, or mechanically weakens in a gastric environment.

Embodiment 293. The method of any of embodiments 221-292, wherein the gastric residence system is used to treat a patient.

Embodiment 294. The method of embodiment 293, wherein the patient is a human.

Embodiment 295. A method for measuring strength of a flexible line of a gastric residence system comprising: receiving a gastric residence system, the gastric residence system comprising: a plurality of arms connected at a proximal end of each arm, the plurality of arms extending radially from the proximal ends; and a flexible line connecting the plurality of arms, wherein a respective portion of the flexible line is embedded within a distal end of each arm; detaching a first arm and a second arm from the gastric residence system, wherein the first arm and the second arm are adjacent arms of the gastric residence system connected by a piece of the flexible line; placing the first arm in a first clamp and the second arm in a second clamp, wherein the first clamp is positioned adjacent to the second clamp; moving the first clamp away from the second clamp until the piece of the flexible line releases from the first arm or the second arm; and measuring the force required to release the piece of the flexible line.

Embodiment 296. The method of embodiment 295, wherein moving the first clamp away from the second clamp causes the flexible line to break.

Embodiment 297. The method of embodiment 295 or 296, further comprising measuring a force required to break the flexible line.

Embodiment 298. A method for measuring strength of a flexible line of a gastric residence system comprising: receiving a gastric residence system, the gastric residence system comprising: a plurality of arms connected at a proximal end of each arm, the plurality of arms extending radially from the proximal ends, at least one arm comprising an active pharmaceutical ingredient-containing portion and a separate tip portion at a distal end of the at least one arm; and a flexible line connecting the plurality of arms, wherein a respective portion of the flexible line is embedded within a distal end of each arm; detaching a first arm and a second arm from the gastric residence system, wherein the first arm and the second arm are adjacent arms of the gastric residence system connected by a piece of the flexible line; placing the first arm in a first clamp and the second arm in a second clamp, wherein the first clamp is positioned adjacent to the second clamp; moving the first clamp away from the second clamp until the piece of the flexible line releases from the first arm or the second arm; and measuring the force required to release the piece of the flexible line.

Embodiment 299. A gastric residence system comprising: six arms affixed to a central elastomer; each arm comprising a proximal end, a distal end, and an outer surface therebetween, wherein the proximal end of each arm is attached to the central elastomer and projects radially from the central elastomer, each arm having its distal end not attached to the central elastomer component and located at a larger radial distance from the central elastomer component than the proximal end; at least one arm comprising an active pharmaceutical ingredient-containing portion, wherein the at least one arm comprises: a first inert segment; a first disintegrating matrix segment attached to the first inert segment; a second inert segment attached to the first disintegrating matrix segment; a second disintegrating matrix segment attached to the second inert segment; a third inert segment attached to the second disintegrating matrix segment; the active pharmaceutical ingredient-containing portion attached to the third inert segment; and a fourth inert segment attached to the active pharmaceutical ingredient-containing portion; and a flexible line connecting each arm, wherein a respective portion of the flexible line is embedded within a distal end of each arm.

Embodiment 300. The gastric residence system of embodiment 299, wherein the first inert segment is attached to the central elastomer.

Embodiment 301. The gastric residence system of embodiment 299 or 300, wherein the segments are in the order listed from the proximal end to the distal end of the arm comprising an active pharmaceutical ingredient-containing portion, wherein the first inert segment is at the proximal end of the arm comprising the active pharmaceutical ingredient-containing portion, the first inert segment is attached to the central elastomer, and the fourth inert segment is at the distal end of the arm comprising the active pharmaceutical ingredient-containing portion.

Embodiment 302. The gastric residence system of any of embodiments 299-301, wherein at least one arm excludes an active pharmaceutical ingredient-containing portion.

Embodiment 303. The gastric residence system of embodiment 302, wherein the at least one arm excluding an active pharmaceutical ingredient-containing portion comprises: a first inert segment; a first disintegrating matrix segment attached to the first inert segment; a second inert segment attached to the first disintegrating matrix segment; a second disintegrating matrix segment attached to the second inert segment; a third inert segment attached to the second disintegrating matrix segment; and a fourth inert segment attached to the third inert segment.

Embodiment 304. The gastric residence system of embodiment 303, wherein the first inert segment is attached to the central elastomer.

Embodiment 305. The gastric residence system of embodiment 303 or 304, wherein the segments are in the order listed from the proximal end to the distal end of the arm excluding an active pharmaceutical ingredient-containing portion, wherein the first inert segment is at the proximal end of the arm excluding an active pharmaceutical ingredient-containing portion, the first inert segment is attached to the central elastomer, and the fourth inert segment is at the distal end of the arm excluding the active pharmaceutical ingredient-containing portion.

Embodiment 306. The gastric residence system of any of embodiments 299-305, wherein one arm comprises the active pharmaceutical ingredient-containing portion and five arms exclude the active pharmaceutical ingredient-containing portion.

Embodiment 307. The gastric residence system of any of embodiments 299-305, wherein two arms comprise the active pharmaceutical ingredient-containing portion and four arms exclude the active pharmaceutical ingredient-containing portion.

Embodiment 308. The gastric residence system of any of embodiments 299-305, wherein three arms comprise the active pharmaceutical ingredient-containing portion and three arms exclude the active pharmaceutical ingredient-containing portion.

Embodiment 309. The gastric residence system of any of embodiments 299-305, wherein four arms comprise the active pharmaceutical ingredient-containing portion and two arms exclude the active pharmaceutical ingredient-containing portion.

Embodiment 310. The gastric residence system of any of embodiments 299-305, wherein five arms comprise the active pharmaceutical ingredient-containing portion and one arm excludes the active pharmaceutical ingredient-containing portion.

Embodiment 311. The gastric residence system of any of embodiments 299-305, wherein all six arms comprise the active pharmaceutical ingredient-containing portion.

Embodiment 312. The gastric residence system of any of embodiments 299-311, wherein the first inert segment comprises PCL.

Embodiment 313. The gastric residence system of any of embodiments 299-312, wherein the first inert segment has a radial length of 0.1-5 mm.

Embodiment 314. The gastric residence system of any of embodiments 299-313, wherein the first inert segment has a radial length of 0.5-3 mm.

Embodiment 315. The gastric residence system of any of embodiments 299-314, wherein the first inert segment has a radial length of 1-1.5 mm.

Embodiment 316. The gastric residence system of any of embodiments 299-315, wherein the first disintegrating matrix segment comprises polycaprolactone.

Embodiment 317. The gastric residence system of embodiment 316, wherein the first disintegrating matrix segment further comprises poly(ethylene oxide).

Embodiment 318. The gastric residence system of embodiment 316 or 317, wherein the first disintegrating matrix segment further comprises DL-lactide/glycolide copolymer.

Embodiment 319. The gastric residence system of any of embodiments 316-318, wherein the first disintegrating matrix segment further comprises ferrosoferric oxide.

Embodiment 320. The gastric residence system of any of embodiments 299-319, wherein the first disintegrating matrix segment has a radial length of 0.1-5 mm.

Embodiment 321. The gastric residence system of any of embodiments 299-320, wherein the first disintegrating matrix segment has a radial length of 0.5-3 mm.

Embodiment 322. The gastric residence system of any of embodiments 299-321, wherein the first disintegrating matrix segment has a radial length of 0.7-1.3 mm.

Embodiment 323. The gastric residence system of any of embodiments 299-322, wherein the second inert segment comprises polycaprolactone.

Embodiment 324. The gastric residence system of embodiment 323, wherein the second inert segment further comprises bismuth subcarbonate.

Embodiment 325. The gastric residence system of any of embodiments 299-324, wherein the second inert segment has a radial length of 0.05-3 mm.

Embodiment 326. The gastric residence system of any of embodiments 299-325, wherein the second inert segment has a radial length of 0.1-1.5 mm.

Embodiment 327. The gastric residence system of any of embodiments 299-326, wherein the second inert segment has a radial length of 0.3-0.7 mm.

Embodiment 328. The gastric residence system of any of embodiments 299-327, wherein the second disintegrating matrix segment comprises polycaprolactone.

Embodiment 329. The gastric residence system of embodiment 328, wherein the second disintegrating matrix segment further comprises HPMCAS-MG.

Embodiment 330. The gastric residence system of embodiment 328 or 329, wherein the second disintegrating matrix segment further comprises poloxamer 407.

Embodiment 331. The gastric residence system of any of embodiments 299-330, wherein the second disintegrating matrix segment has a radial length of 0.1-5 mm.

Embodiment 332. The gastric residence system of any of embodiments 299-331, wherein the second disintegrating matrix segment has a radial length of 0.5-3 mm.

Embodiment 333. The gastric residence system of any of embodiments 29-332, wherein the second disintegrating matrix segment has a radial length of 1.5-2.5 mm.

Embodiment 334. The gastric residence system of any of embodiments 299-333, wherein the third inert segment comprises polycaprolactone.

Embodiment 335. The gastric residence system of embodiment 334, wherein the third inert segment further comprises bismuth subcarbonate.

Embodiment 336. The gastric residence system of any of embodiments 299-335, wherein the third inert segment has a radial length of 0.05-3 mm.

Embodiment 337. The gastric residence system of any of embodiments 299-336, wherein the third inert segment has a radial length of 0.1-1.5 mm.

Embodiment 338. The gastric residence system of any of embodiments 299-337, wherein the third inert segment has a radial length of 0.3-0.7 mm.

Embodiment 339. The gastric residence system of any of embodiments 299-338, wherein the fourth inert segment comprises polycaprolactone.

Embodiment 340. The gastric residence system of embodiment 339, wherein the fourth inert segment further comprises copovidone.

Embodiment 341. The gastric residence system of embodiment 339 or 340, wherein the fourth inert segment further comprises poloxamer 407.

Embodiment 342. The gastric residence system of any of embodiments 339-341, wherein the fourth inert segment further comprises a colorant.

Embodiment 343. The gastric residence system of any of embodiments 299-342, wherein the fourth inert segment has a radial length of 2-20 mm.

Embodiment 344. The gastric residence system of any of embodiments 299-343, wherein the fourth inert segment has a radial length of 4-15 mm.

Embodiment 345. The gastric residence system of any of embodiments 299-344, wherein the fourth inert segment has a radial length of 4.5-13.5 mm.

Embodiment 346. The gastric residence system of any of embodiments 299-345, wherein the fourth inert segment has a radial length of 12.1-13.3 mm.

Embodiment 347. The gastric residence system of any of embodiments 299-345, wherein the fourth inert segment has a radial length of 4.8-6 mm.

Embodiment 348. The gastric residence system of any of embodiments 299-347, wherein the active pharmaceutical ingredient-containing portion comprises risperidone or a pharmaceutically acceptable salt thereof.

Embodiment 349. The gastric residence system of embodiment 348, wherein the active pharmaceutical ingredient-containing portion further comprises polycaprolactone.

Embodiment 350. The gastric residence system of embodiment 348 or 349, wherein the active pharmaceutical ingredient-containing portion further comprises copovidone.

Embodiment 351. The gastric residence system of any of embodiments 348-350, wherein the active pharmaceutical ingredient-containing portion further comprises poloxamer 407.

Embodiment 352. The gastric residence system of any of embodiments 348-351, wherein the active pharmaceutical ingredient-containing portion further comprises vitamin E succinate.

Embodiment 353. The gastric residence system of any of embodiments 348-352, wherein the active pharmaceutical ingredient-containing portion further comprises silicon dioxide.

Embodiment 354. The gastric residence system of any of embodiments 348-353, wherein the active pharmaceutical ingredient-containing portion further comprises one or more colorants.

Embodiment 355. The gastric residence system of any of embodiments 299-354, wherein the active pharmaceutical ingredient-containing portion has a radial length of 3-12 mm.

Embodiment 356. The gastric residence system of any of embodiments 299-355, wherein the active pharmaceutical ingredient-containing portion has a radial length of 5-9 mm.

Embodiment 357. The gastric residence system of any of embodiments 299-356, wherein the active pharmaceutical ingredient-containing portion has a radial length of 6.5-8.5 mm.

Embodiment 358. The gastric residence system of any of embodiments 299-357, wherein the active pharmaceutical ingredient-containing portion comprises 10-80% by weight of an active pharmaceutical ingredient.

Embodiment 359. The gastric residence system of any of embodiments 299-358, wherein the active pharmaceutical ingredient-containing portion comprises 40-60% by weight of an active pharmaceutical ingredient.

Embodiment 360. The gastric residence system of any of embodiments 299-358, wherein the active pharmaceutical ingredient-containing portion comprises 20-40% by weight of an active pharmaceutical ingredient.

Embodiment 361. The gastric residence system of any of embodiments 299-360, wherein the at least one arm comprising an active pharmaceutical ingredient-containing portion comprises a coating.

Embodiment 362. The gastric residence system of embodiment 361, wherein the coating comprises polycaprolactone.

Embodiment 363. The gastric residence system of embodiment 362, wherein the coating further comprises copovidone.

Embodiment 364. The gastric residence system of embodiment 362 or 363, wherein the coating further comprises magnesium stearate.

Embodiment 365. The gastric residence system of any of embodiments 299-364, wherein the gastric residence system has a maximum diameter of 40-50 mm.

Embodiment 366. The gastric residence system of any of embodiments 299-365, wherein a respective portion of the flexible line connecting each arm is embedded in the fourth inert segment of each arm.

Embodiment 367. The gastric residence system of any of embodiments 299-366, wherein the flexible line is embedded in the distal end of each arm by heating the distal end, pressing a portion of the flexible line into the distal end, and cooling the distal end with the portion of the flexible line embedded.

Embodiment 368. The gastric residence system of embodiment 367, wherein heating the distal end comprises directing a laser at the distal end.

Embodiment 369. The gastric residence system of embodiment 368, wherein the laser comprises an infrared laser.

Embodiment 370. The gastric residence system of any of embodiments 367-369, wherein heating comprises convection heating.

Embodiment 371. The gastric residence system of any of embodiments 299-370, wherein the flexible line comprises an irregular cross-section.

Embodiment 372. The gastric residence system of embodiment 371, wherein the irregular cross-section comprises a braided cross-section.

Embodiment 373. The gastric residence system of embodiment 371 or 372, wherein the irregular cross-section comprises at least one of bumps, ridges, knots, barbs, or valleys.

Embodiment 374. The gastric residence system of any of embodiments 299-373, wherein each arm comprises a triangular cross-section.

Embodiment 375. The gastric residence system of embodiment 374, wherein the triangular cross-section has a base of 2.5-3.5 mm and a height of 2.1-3.1 mm.

Embodiment 376. The gastric residence system of embodiment 374, wherein the triangular cross-section has a base of 2.9-3.3 mm and a height of 2.5-2.9 mm.

Embodiment 377. The gastric residence system of any of embodiments 299-376, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

Embodiment 378. The gastric residence system of any of embodiments 299-377, wherein the flexible line comprises one or more of poly(glycolic acid) (PGA), poly(lactic-co-glycolic acid) (PLGA), or polyethylene terephthalate (PET).

Embodiment 379. The gastric residence system of any of embodiments 299-378, wherein the flexible line comprises PLGA in a poly(glycolic acid) (PGA) to poly(lactic acid) (PLA) ratio of 95:5 to 40:60.

Embodiment 380. The gastric residence system of any of embodiments 299-379, wherein the flexible line comprises PLGA with an intrinsic viscosity of 0.5-3 dL/g.

Embodiment 381. The gastric residence system of any of embodiments 299-380, wherein the flexible line comprises at least one crystalline material.

Embodiment 382. The gastric residence system of any of embodiments 299-381, wherein the flexible line comprises at least one semi-crystalline material.

Embodiment 383. The gastric residence system of any of embodiments 299-382, wherein the flexible line comprises at least one amorphous material.

Embodiment 384. The gastric residence system of any of embodiments 299-383, wherein the flexible line is configured to degrade in less than 14 days.

Embodiment 385. The gastric residence system of any of embodiments 299-384, wherein the flexible line is configured to degrade in less than 10 days.

Embodiment 386. The gastric residence system of any of embodiments 299-385, wherein the flexible line is a sterilized flexible line.

Embodiment 387. The gastric residence system of any of embodiments 299-386, wherein the flexible line has a diameter of 0.05-1 mm.

Embodiment 388. The gastric residence system of any of embodiments 299-387, wherein the flexible line has a cross-sectional area of 0.005-3.14 mm².

Embodiment 389. The gastric residence system of any of embodiments 299-387, wherein the flexible line has a cross-sectional area of 0.06-0.13 mm².

Embodiment 390. The gastric residence system of any of embodiments 299-387, wherein the flexible line has a cross-sectional area of 0.1-5 mm².

Embodiment 391. The gastric residence system of any of embodiments 299-387, wherein the flexible line has a cross-sectional area of 0.5-3.5 mm².

Embodiment 392. The gastric residence system of any of embodiments 299-387, wherein the flexible line has a cross-sectional area of 0.6-1 mm².

Embodiment 393. The gastric residence system of any of embodiments 299-392, wherein the flexible line has a surface roughness of 5-400 μm.

Embodiment 394. The gastric residence system of any of embodiments 299-392, wherein the flexible line has a surface roughness of 50-250 μm.

Embodiment 395. The gastric residence system of any of embodiments 299-394, wherein the flexible line is absorbable.

Embodiment 396. The gastric residence system of any of embodiments 299-394, wherein the flexible line is non-absorbable.

Embodiment 397. The gastric residence system of any of embodiments 299-396, wherein the flexible line comprises a coating.

Embodiment 398. The gastric residence system of embodiment 397, wherein the coating comprises polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

1. A gastric residence system comprising:
a plurality of arms connected at a proximal end of each arm, the plurality of arms extending radially from the proximal ends, at least one arm comprising an active pharmaceutical ingredient-containing portion and a separate tip portion at a distal end of the at least one arm; and
a flexible line connecting the plurality of arms, wherein a respective portion of the flexible line is embedded within a distal end of each arm, wherein the flexible line comprises an irregular cross-section.

2. The gastric residence system of claim 1, wherein the irregular cross-section comprises a braided cross-section.

3. The gastric residence system of claim 1, wherein the irregular cross-section comprises at least one of bumps, ridges, knots, barbs, or valleys.

4. The gastric residence system of claim 1, wherein the active pharmaceutical ingredient-containing portion comprises 10-80% by weight of an active pharmaceutical ingredient.

5. The gastric residence system of claim 1, wherein each arm of the plurality of arms comprises a triangular cross-section.

6. The gastric residence system of claim 5, wherein the triangular cross-section has a base of 2.5-3.5 mm and a height of 2.1-3.1 mm.

7. The gastric residence system of claim 1, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

8. The gastric residence system of claim 1, wherein the flexible line comprises PLGA in a poly(glycolic acid) (PGA) to poly(lactic acid) (PLA) ratio of 95:5 to 40:60.

9. The gastric residence system of claim 1, wherein the flexible line comprises PLGA with an intrinsic viscosity of 0.5-3 dL/g.

10. The gastric residence system of claim 1, wherein the flexible line is configured to degrade in less than 14 days.

11. The gastric residence system of claim 1, wherein the flexible line has a diameter of 0.05-1 mm.

12. The gastric residence system of claim 1, wherein the flexible line has a cross-sectional area of 0.005-3.14 mm².

13. The gastric residence system of claim 1, wherein the flexible line has a surface roughness of 5-400 μm.

14. The gastric residence system of claim 1, wherein the flexible line comprises a braid of at least three strands.

15. The gastric residence system of claim 14, wherein the braid has a braid angle of 5-40 degrees and a braid length of 0.5-3.1 mm.

16. The gastric residence system of claim 1, wherein the flexible line is absorbable.

17. The gastric residence system of claim 1, wherein the flexible line is non-absorbable.

18. The gastric residence system of claim 1, wherein the flexible line comprises a coating comprising polycaprolactone (PCL), polycaprolate, polyglactin 370, polyhydroxybutyrate, poly(L-lactide co-caprolactone), poly(L-lactide), poly(L-lactide-co-D,L lactide), polyglycolide, poly(L-lactide-co-glycolide), poly(DL-lactide), poly(D,L-lactide-co-glycolide), thermoplastic polyurethanes, poloxamer 188, or a combination thereof.

19. The gastric residence system of claim 1, wherein the separate tip portion and/or the distal end of each arm comprises polycaprolactone.

20. The gastric residence system of claim 19, wherein the separate tip portion and/or the distal end of each arm further comprises at least one of copovidone, Poloxamer 407, a colorant, and bismuth subcarbonate.

21. The gastric residence system of claim 1, wherein the separate tip portion and/or the distal end of each arm has a flexural modulus of at least 50 MPa.

22. The gastric residence system of claim 1, wherein the separate tip portion and/or the distal end of each arm has a flexural strength of at least 5 N.

23. The gastric residence system of claim 1, wherein the separate tip portion and/or the distal end of each arm has a maximum force of at least 3 N.

24. The gastric residence system of claim 1, wherein a force required to release the flexible line from a first arm of the plurality of arms is at least 4N.

25. A gastric residence system comprising:
a plurality of arms connected at a proximal end of each arm, the plurality of arms extending radially from the proximal ends, at least one arm comprising an active pharmaceutical ingredient-containing portion and a separate tip portion at a distal end of the at least one arm; and
a flexible line connecting the plurality of arms, wherein a respective portion of the flexible line is embedded within a distal end of each arm, wherein the flexible line comprises one or more of poly(glycolic acid), poly(lactic-co-glycolic acid), poly(glycolide/lactide) random copolymer, poly-p-dioxanone, poly(glycolide/trimethylene carbonate) block copolymer, poly(glycolide/ε-caprolactone), poly(gycolide/p-dioxanone/trimethylene carbonate) triblock copolymer, poly(glycolide/ε-caprolactone/trimethylene carbonate) triblock copolymer, poly(glycolide/L-lactide/ε-caprolactone/trimethylene carbonate) triblock copolymer, polyglytone 6211, 100% poly-L-lactide, polyhydroxyalkanoates (PHA), poly(ethylene glycol)-co-poly(lactic acid) (PELA), polyethylene terephthalate (PET), polypropylene (PP), ultra-high molecular weight polyethylene (UHMWPE), polyamide, polyester, poly(ether ester), polytetrafluoroethylene (PTFE), or polyvinylidinefluoride (PVDF).

26. A gastric residence system comprising:
a plurality of arms connected at a proximal end of each arm, the plurality of arms extending radially from the proximal ends, at least one arm comprising an active pharmaceutical ingredient-containing portion; and
a flexible line connecting the plurality of arms, wherein a respective portion of the flexible line is embedded within a distal end of each arm, wherein the flexible line comprises an irregular cross-section.

27. A method for manufacturing a gastric residence system comprising:
using a mold, preparing a gastric residence system comprising a plurality of arms connected at a proximal end of each arm, the plurality of arms extending radially from the proximal ends;
heating a distal end of each arm to at least partially melt the respective distal end;
embedding a respective portion of at least one flexible line into each melted distal end; and
cooling the melted distal ends such that melted distal ends solidify with the at least one flexible line embedded.

28. The method of claim 27, wherein heating the distal end of each arm comprises directing a laser at the distal end of each arm or convection heating the distal end of each arm.

29. A method for measuring strength of a flexible line of a gastric residence system comprising:
receiving a gastric residence system, the gastric residence system comprising:
a plurality of arms connected at a proximal end of each arm, the plurality of arms extending radially from the proximal ends; and
a flexible line connecting the plurality of arms, wherein a respective portion of the flexible line is embedded within a distal end of each arm;
detaching a first arm and a second arm from the gastric residence system, wherein the first arm and the second arm are adjacent arms of the gastric residence system connected by a piece of the flexible line;
placing the first arm in a first clamp and the second arm in a second clamp, wherein the first clamp is positioned adjacent to the second clamp;
moving the first clamp away from the second clamp until the piece of the flexible line releases from the first arm or the second arm; and
measuring the force required to release the piece of the flexible line.

30. A gastric residence system comprising:
six arms affixed to a central elastomer;
each arm comprising a proximal end, a distal end, and an outer surface therebetween, wherein the proximal end of each arm is attached to the central elastomer and projects radially from the central elastomer, each arm having its distal end not attached to the central elastomer component and located at a larger radial distance from the central elastomer component than the proximal end;
at least one arm comprising an active pharmaceutical ingredient-containing portion, wherein the at least one arm comprises:
a first inert segment;
a first disintegrating matrix segment attached to the first inert segment;
a second inert segment attached to the first disintegrating matrix segment;
a second disintegrating matrix segment attached to the second inert segment;
a third inert segment attached to the second disintegrating matrix segment;
the active pharmaceutical ingredient-containing portion attached to the third inert segment; and
a fourth inert segment attached to the active pharmaceutical ingredient-containing portion;
and
a flexible line connecting each arm, wherein a respective portion of the flexible line is embedded within a distal end of each arm.
